SEQUENCE LISTING (423 sequences)

SEQ ID NO: 1 - protein, 13 aa, synthetic construct
WHWLQFWRGQSMY

SEQ ID NO: 2 - protein, 13 aa, synthetic construct
PHWGSFKKGQPMY

SEQ ID NO: 3 - protein, 13 aa, synthetic construct
KHWVTLKWGAPMY

SEQ ID NO: 4 - protein, 13 aa, synthetic construct
WHLLRFKPGQPMY

SEQ ID NO: 5 - protein, 13 aa, synthetic construct
PHWLRFKPGQPMY

SEQ ID NO: 6 - protein, 13 aa, synthetic construct
PHWVQLKRGAPMY

SEQ ID NO: 7 - protein, 13 aa, synthetic construct
KHWLQIKYGEPMY

SEQ ID NO: 8 - protein, 13 aa, synthetic construct
KHWIKLMPGQGMY

SEQ ID NO: 9 - protein, 13 aa, synthetic construct
KHWLRFKPGQPMY

SEQ ID NO: 10 - protein, 13 aa, synthetic construct
PHWVQIKLGQPMY

SEQ ID NO: 11 - protein, 13 aa, synthetic construct
KHWITFKPGQPMY

SEQ ID NO: 12 - protein, 13 aa, synthetic construct
KHVLRFKPGQPMY

SEQ ID NO: 13 - protein, 13 aa, synthetic construct
KHWLKFKPGQPMY

SEQ ID NO: 14 - protein, 13 aa, synthetic construct
KHWLKLWPGQPMY

SEQ ID NO: 15 - protein, 13 aa, synthetic construct
WHTLKIKKGEPMY

SEQ ID NO: 16 - protein, 13 aa, synthetic construct
WHWLTFKTGQGMY

SEQ ID NO: 17 - protein, 13 aa, synthetic construct
KHFLKLWPGQPMY

SEQ ID NO: 18 - protein, 13 aa, synthetic construct
KHFLRFKPGQPMY

SEQ ID NO: 19 - protein, 13 aa, synthetic construct
PKWIRLKPGQPMY

SEQ ID NO: 20 - protein, 13 aa, synthetic construct
KHWLSFKPGQPMY

SEQ ID NO: 21 - protein, 13 aa, synthetic construct
KHWVQLMKGQPMY

SEQ ID NO: 22 - protein, 13 aa, synthetic construct
KHLVQFKMGQPMY

SEQ ID NO: 23 - protein, 13 aa, synthetic construct
WHWVALKNGQGMY

SEQ ID NO: 24 - protein, 13 aa, synthetic construct
KHYLNFKPGQPMY

SEQ ID NO: 25 - protein, 13 aa, synthetic construct
WHLLQFKVGQPMY

SEQ ID NO: 26 - protein, 13 aa, synthetic construct
WHWIQFKPGQSMY

SEQ ID NO: 27 - protein, 13 aa, synthetic construct
KHFLNLKSGQPMY

SEQ ID NO: 28 - protein, 13 aa, synthetic construct
KHFLALKPGQPMY

SEQ ID NO: 29 - protein, 13 aa, synthetic construct
KHDLRFKPGQPMY

SEQ ID NO: 30 - protein, 13 aa, synthetic construct
PHWLTIKPGQPMY

SEQ ID NO: 31 - protein, 13 aa, synthetic construct
KEWIQLKLGQPMY

SEQ ID NO: 32 - protein, 13 aa, synthetic construct
KSWLSLKPGQGMY

SEQ ID NO: 33 - protein, 13 aa, synthetic construct
WRWGQFKPGEPMY

SEQ ID NO: 34 - protein, 13 aa, synthetic construct
PHWLSFFPGQGMY

SEQ ID NO: 35 - protein, 13 aa, synthetic construct
KCWLELKTGMPMY

SEQ ID NO: 36 - protein, 13 aa, synthetic construct
WHMLEFKPGQPMY

SEQ ID NO: 37 - protein, 13 aa, synthetic construct
PHWIQLFNGQPMY

SEQ ID NO: 38 - protein, 13 aa, synthetic construct
KHVLQFKPGEPMY

SEQ ID NO: 39 - protein, 13 aa, synthetic construct
KSWGQFKPGQPMY

SEQ ID NO: 40 - protein, 13 aa, synthetic construct
PEWIQLKTGMPMY

SEQ ID NO: 41 - protein, 13 aa, synthetic construct
PHLLNFKPGQPMY

SEQ ID NO: 42 - protein, 13 aa, synthetic construct
PHSGQLWSGQPMY

SEQ ID NO: 43 - protein, 13 aa, synthetic construct
PHWGQFKLGQPMY

SEQ ID NO: 44 - protein, 13 aa, synthetic construct
PHWVNLKTGQGMY

SEQ ID NO: 45 - protein, 13 aa, synthetic construct
PTGLQFKPGQSMY

SEQ ID NO: 46 - protein, 13 aa, synthetic construct
WHWLSLNTGQPMY

SEQ ID NO: 47 - protein, 13 aa, synthetic construct
KHDLELWPGQPMY

SEQ ID NO: 48 - protein, 13 aa, synthetic construct
WGGLALKPGQPMY

SEQ ID NO: 49 - protein, 13 aa, synthetic construct
PEWLKLYPGQPMY

SEQ ID NO: 50 - protein, 13 aa, synthetic construct
WRVLQLKPGSSMY

SEQ ID NO: 51 - protein, 13 aa, synthetic construct
WHWVRIWPGQPMY

SEQ ID NO: 52 - protein, 13 aa, synthetic construct
KHWLRIKWGEPMY

SEQ ID NO: 53 - protein, 13 aa, synthetic construct
WKWVRLKTGQPMY

SEQ ID NO: 54 - protein, 13 aa, synthetic construct
KHMIQLYWGQPMY

SEQ ID NO: 55 - protein, 13 aa, synthetic construct
WRYLRLKPGSGMY

SEQ ID NO: 56 - protein, 13 aa, synthetic construct
WHSLKLKRGQPMY

SEQ ID NO: 57 - protein, 13 aa, synthetic construct
WHWLAIFRGQPMY

SEQ ID NO: 58 - protein, 13 aa, synthetic construct
WKWVQIKKGAPMY

SEQ ID NO: 59 - protein, 13 aa, synthetic construct
KHWITLKSGMPMY

SEQ ID NO: 60 - protein, 13 aa, synthetic construct
WKTLQLKIGAPMY

SEQ ID NO: 61 - protein, 13 aa, synthetic construct
KHWLKLFPGQPMY

SEQ ID NO: 62 - protein, 13 aa, synthetic construct
KHWLKIKIGEPMY

SEQ ID NO: 63 - protein, 13 aa, synthetic construct
PRWLQIKKGQSMY

SEQ ID NO: 64 - protein, 13 aa, synthetic construct
KHWLQFKPGSGMY

SEQ ID NO: 65 - protein, 13 aa, synthetic construct
WKWVNLRTGQPMY

SEQ ID NO: 66 - protein, 13 aa, synthetic construct
PHWISLKKGQPMY

SEQ ID NO: 67 - protein, 13 aa, synthetic construct
KHMLRIKPGQPMY

SEQ ID NO: 68 - protein, 13 aa, synthetic construct
KHWGRLRAGQPMY

SEQ ID NO: 69 - protein, 13 aa, synthetic construct
KHWLRLNPGQPMY

SEQ ID NO: 70 - protein, 13 aa, synthetic construct
WHYLRLKPGQSMY

SEQ ID NO: 71 - protein, 13 aa, synthetic construct
WHWIQLKKGEPMY

SEQ ID NO: 72 - protein, 13 aa, synthetic construct
KHYLKFKPGQPMY

SEQ ID NO: 73 - protein, 13 aa, synthetic construct
WQWLRIKSGQGMY

SEQ ID NO: 74 - protein, 13 aa, synthetic construct
WHFVKLHPGQPMY

SEQ ID NO: 75 - protein, 13 aa, synthetic construct
KHWIRLWPGQPMY

SEQ ID NO: 76 - protein, 13 aa, synthetic construct
WHWLRLKPGASMY

SEQ ID NO: 77 - protein, 13 aa, synthetic construct
PHWIKLHTGQPMY

SEQ ID NO: 78 - protein, 13 aa, synthetic construct
KHFLNFKPGQPMY

SEQ ID NO: 79 - protein, 13 aa, synthetic construct
WRDIRLKKGQPMY

SEQ ID NO: 80 - protein, 13 aa, synthetic construct
KHTLRFKPGQPMY

SEQ ID NO: 81 - protein, 13 aa, synthetic construct
KHWIKLKNGQPMY

SEQ ID NO: 82 - protein, 13 aa, synthetic construct
WHWGSLKVGQPMY

SEQ ID NO: 83 - protein, 13 aa, synthetic construct
KHTLRLKPGQPMY

SEQ ID NO: 84 - protein, 13 aa, synthetic construct
KHMLRFKPGQPMY

SEQ ID NO: 85 - protein, 13 aa, synthetic construct
WHFLKFKPGEPMY

SEQ ID NO: 86 - protein, 13 aa, synthetic construct
KHFLKIKPGQPMY

SEQ ID NO: 87 - protein, 13 aa, synthetic construct
KHVLKFKPGQPMY

SEQ ID NO: 88 - protein, 13 aa, synthetic construct
KHLLRLKPGQPMY

SEQ ID NO: 89 - protein, 13 aa, synthetic construct
WHSLEFKIGQGMY

SEQ ID NO: 90 - protein, 13 aa, synthetic construct
WHFLQIKPGQGMY

SEQ ID NO: 91 - protein, 13 aa, synthetic construct
KQWLKLKNGQPMY

SEQ ID NO: 92 - protein, 13 aa, synthetic construct
WHTLRFKPGQPMY

SEQ ID NO: 93 - protein, 13 aa, synthetic construct
KHTLQLKRGQPMY

SEQ ID NO: 94 - protein, 13 aa, synthetic construct
KHYLSFKPGQPMY

SEQ ID NO: 95 - protein, 13 aa, synthetic construct
KHLLTFKPGQPMY

SEQ ID NO: 96 - protein, 13 aa, synthetic construct
KHFLTLKPGQPMY

SEQ ID NO: 97 - protein, 13 aa, synthetic construct
WHWVQFRPGAPMY

SEQ ID NO: 98 - protein, 13 aa, synthetic construct
WKWGSLKNGQPMY

SEQ ID NO: 99 - protein, 13 aa, synthetic construct
WHWLKIWPGQPMY

SEQ ID NO: 100 - protein, 13 aa, synthetic construct
WKYLNFKPGQPMY

SEQ ID NO: 101 - protein, 13 aa, synthetic construct
WHVLRFKPGQPMY

SEQ ID NO: 102 - protein, 13 aa, synthetic construct
KHYLTLKPGQPMY

SEQ ID NO: 103 - protein, 13 aa, synthetic construct
KHYLALKPGQPMY

SEQ ID NO: 104 - protein, 13 aa, synthetic construct
KRGINLKPGQPMY

SEQ ID NO: 105 - protein, 13 aa, synthetic construct
WHILRFKNGMPMY

SEQ ID NO: 106 - protein, 13 aa, synthetic construct
WHWVQLKKGEPMY

SEQ ID NO: 107 - protein, 13 aa, synthetic construct
KHFLSLKPGQPMY

SEQ ID NO: 108 - protein, 13 aa, synthetic construct
WHWLKLNYGQPMY

SEQ ID NO: 109 - protein, 13 aa, synthetic construct
KRWLQIRPGEPMY

SEQ ID NO: 110 - protein, 13 aa, synthetic construct
WHWLSIKPGQGMY

SEQ ID NO: 111 - protein, 13 aa, synthetic construct
WHWVQFGPGSPMY

SEQ ID NO: 112 - protein, 13 aa, synthetic construct
PHFLRLKPGQPMY

SEQ ID NO: 113 - protein, 13 aa, synthetic construct
KHLLNLKPGQPMY

SEQ ID NO: 114 - protein, 13 aa, synthetic construct
WHWINLWPGQPMY

SEQ ID NO: 115 - protein, 13 aa, synthetic construct
KKVLQLKLGQPMY

SEQ ID NO: 116 - protein, 13 aa, synthetic construct
KHDLKFKPGQPMY

SEQ ID NO: 117 - protein, 13 aa, synthetic construct
WQSGQLKPGQPMY

SEQ ID NO: 118 - protein, 13 aa, synthetic construct
WHMLNFKPGQPMY

SEQ ID NO: 119 - protein, 13 aa, synthetic construct
WHWLKLGTGEPMY

SEQ ID NO: 120 - protein, 13 aa, synthetic construct
WSMGQIKPGSPMY

SEQ ID NO: 121 - protein, 13 aa, synthetic construct
PSGLQIKVGQPMY

SEQ ID NO: 122 - protein, 13 aa, synthetic construct
PEWLSLKPGSSMY

SEQ ID NO: 123 - protein, 13 aa, synthetic construct
KQWVQLKYGQGMY

SEQ ID NO: 124 - protein, 13 aa, synthetic construct
WHGIQLFKGQPMY

SEQ ID NO: 125 - protein, 13 aa, synthetic construct
KEWVQLYPGQPMY

SEQ ID NO: 126 - protein, 13 aa, synthetic construct
PHDLEFKPGQGMY

SEQ ID NO: 127 - protein, 13 aa, synthetic construct
WKSINFKPGQPMY

SEQ ID NO: 128 - protein, 13 aa, synthetic construct
PHWVEFDPGQPMY

SEQ ID NO: 129 - protein, 13 aa, synthetic construct
WQGLELHPGQPMY

SEQ ID NO: 130 - protein, 13 aa, synthetic construct
WEVLTLKPGSPMY

SEQ ID NO: 131 - protein, 13 aa, synthetic construct
PHLIQLGLGQPMY

SEQ ID NO: 132 - protein, 13 aa, synthetic construct
PHMVRLKPGQPMY

SEQ ID NO: 133 - protein, 13 aa, synthetic construct
KGWGQLIPGEPMY

SEQ ID NO: 134 - protein, 13 aa, synthetic construct
WGDLKLKPGQPMY

SEQ ID NO: 135 - protein, 13 aa, synthetic construct
WHWGQLQRGEPMY

SEQ ID NO: 136 - protein, 13 aa, synthetic construct
PHWLKLKPGEPMY

SEQ ID NO: 137 - protein, 13 aa, synthetic construct
WHMGQLKPGEPMY

SEQ ID NO: 138 - protein, 13 aa, synthetic construct
WQMLKLKYGQPMY

SEQ ID NO: 139 - protein, 13 aa, synthetic construct
PHWGEIKPGQSMY

SEQ ID NO: 140 - protein, 13 aa, synthetic construct
KCFIQLKPGQPMY

SEQ ID NO: 141 - protein, 13 aa, synthetic construct
PRDLTLKRGQPMY

SEQ ID NO: 142 - protein, 13 aa, synthetic construct
PHGLSIYPGQPMY

SEQ ID NO: 143 - protein, 13 aa, synthetic construct
PHWGELKPGQPMY

SEQ ID NO: 144 - protein, 13 aa, synthetic construct
KHWVQLFPGQPMY

SEQ ID NO: 145 - protein, 13 aa, synthetic construct
KEWLNFKNGQPMY

SEQ ID NO: 146 - protein, 13 aa, synthetic construct
WRTGQFWPGQPMY

SEQ ID NO: 147 - protein, 13 aa, synthetic construct
PHDGQLKRGQPMY

SEQ ID NO: 148 - protein, 13 aa, synthetic construct
PKWLNLKPGEPMY

SEQ ID NO: 149 - protein, 13 aa, synthetic construct
WRVLKLMPGQPMY

SEQ ID NO: 150 - protein, 13 aa, synthetic construct
WHFLQIKPGEPMY

SEQ ID NO: 151 - protein, 13 aa, synthetic construct
KEWLNLKPGQPMY

SEQ ID NO: 152 - protein, 13 aa, synthetic construct
KSWLQIKNGQPMY

SEQ ID NO: 153 - protein, 13 aa, synthetic construct
KGILTLKPGQPMY

SEQ ID NO: 154 - protein, 13 aa, synthetic construct
KHVLALKPGSPMY

SEQ ID NO: 155 - protein, 13 aa, synthetic construct
WKILTIKPGQPMY

SEQ ID NO: 156 - protein, 13 aa, synthetic construct
KHDVQLKNGSPMY

SEQ ID NO: 157 - protein, 13 aa, synthetic construct
WHWGNFQTGQPMY

SEQ ID NO: 158 - protein, 13 aa, synthetic construct
PHWLNLKPGEPMY

SEQ ID NO: 159 - protein, 13 aa, synthetic construct
WKSLNFKPGQSMY

SEQ ID NO: 160 - protein, 13 aa, synthetic construct
PHWVSLKPGQPMY

SEQ ID NO: 161 - protein, 13 aa, synthetic construct
PHWVAFKPGSPMY

SEQ ID NO: 162 - protein, 13 aa, synthetic construct
KHYLELKPGQPMY

SEQ ID NO: 163 - protein, 13 aa, synthetic construct
WHMVQLKPGEPMY

SEQ ID NO: 164 - protein, 13 aa, synthetic construct
KGTLSLKPGQPMY

SEQ ID NO: 165 - protein, 13 aa, synthetic construct
KHSLEFKPGQPMY

SEQ ID NO: 166 - protein, 13 aa, synthetic construct
WEFVQFKRGQPMY

SEQ ID NO: 167 - protein, 13 aa, synthetic construct
WHWLEFQRGQPMY

SEQ ID NO: 168 - protein, 13 aa, synthetic construct
KHLVQLHPGQPMY

SEQ ID NO: 169 - protein, 13 aa, synthetic construct
PHTIQLWKGQPMY

SEQ ID NO: 170 - protein, 13 aa, synthetic construct
WHMLTFKPGQPMY

SEQ ID NO: 171 - protein, 13 aa, synthetic construct
WHLGQFKPGEPMY

SEQ ID NO: 172 - protein, 13 aa, synthetic construct
WSWGALKSGMPMY

SEQ ID NO: 173 - protein, 13 aa, synthetic construct
PHWVQLKNGQPMY

SEQ ID NO: 174 - protein, 13 aa, synthetic construct
WHSVQLHPGQPMY

SEQ ID NO: 175 - protein, 13 aa, synthetic construct
KKWIQLKPGEPMY

SEQ ID NO: 176 - protein, 13 aa, synthetic construct
WHDVNLKRGSPMY

SEQ ID NO: 177 - protein, 13 aa, synthetic construct
KHTLEFKPGQPMY

SEQ ID NO: 178 - protein, 13 aa, synthetic construct
WHSVQFWPGQPMY

SEQ ID NO: 179 - protein, 13 aa, synthetic construct
KHFLELKPGQPMY

SEQ ID NO: 180 - protein, 13 aa, synthetic construct
KHDLALKIGQPMY

SEQ ID NO: 181 - protein, 13 aa, synthetic construct
KCWLNLKAGQGMY

SEQ ID NO: 182 - protein, 13 aa, synthetic construct
KEWLQLNWGQPMY

SEQ ID NO: 183 - protein, 13 aa, synthetic construct
KHLLNFKPGQPMY

SEQ ID NO: 184 - protein, 13 aa, synthetic construct
KHMLKLKIGEPMY

SEQ ID NO: 185 - protein, 13 aa, synthetic construct
KHMLQFSMGQPMY

SEQ ID NO: 186 - protein, 13 aa, synthetic construct
KHMLQLKPGAGMY

SEQ ID NO: 187 - protein, 13 aa, synthetic construct
KHVLRFKPGQPMY

SEQ ID NO: 188 - protein, 13 aa, synthetic construct
KHWIKLKPGQPMY

SEQ ID NO: 189 - protein, 13 aa, synthetic construct
KHWIQIKPGQSMY

SEQ ID NO: 190 - protein, 13 aa, synthetic construct
KHWLQFNMGAPMY

SEQ ID NO: 191 - protein, 13 aa, synthetic construct
KHWVQLNPGQPMY

SEQ ID NO: 192 - protein, 13 aa, synthetic construct
KQYLQIKVGQPMY

SEQ ID NO: 193 - protein, 13 aa, synthetic construct
KSWLELKIGQPMY

SEQ ID NO: 194 - protein, 13 aa, synthetic construct
KSWVQLKAGEPMY

SEQ ID NO: 195 - protein, 13 aa, synthetic construct
KTWLQLNLGEPMY

SEQ ID NO: 196 - protein, 13 aa, synthetic construct
PHLLEFKPGQPMY

SEQ ID NO: 197 - protein, 13 aa, synthetic construct
PHLLQFKVGEPMY

SEQ ID NO: 198 - protein, 13 aa, synthetic construct
PHMLQIKYGQGMY

SEQ ID NO: 199 - protein, 13 aa, synthetic construct
PHSLQFKPGQGMY

SEQ ID NO: 200 - protein, 13 aa, synthetic construct
PHWIQFKKGQPMY

SEQ ID NO: 201 - protein, 13 aa, synthetic construct
PHWIQLFAGQPMY

SEQ ID NO: 202 - protein, 13 aa, synthetic construct
PHWIQLSLGQPMY

SEQ ID NO: 203 - protein, 13 aa, synthetic construct
PHWLQFGKGQPMY

SEQ ID NO: 204 - protein, 13 aa, synthetic construct
PHWLSIYWGQPMY

SEQ ID NO: 205 - protein, 13 aa, synthetic construct
PHWLTLNYGMPMY

SEQ ID NO: 206 - protein, 13 aa, synthetic construct
WCVLQIKWGMPMY

SEQ ID NO: 207 - protein, 13 aa, synthetic construct
WCWGQLKIGEPMY

SEQ ID NO: 208 - protein, 13 aa, synthetic construct
WCWLNLYNGQPMY

SEQ ID NO: 209 - protein, 13 aa, synthetic construct
WCWVQFKPGQSMY

SEQ ID NO: 210 - protein, 13 aa, synthetic construct
WEWIALNPGMPMY

SEQ ID NO: 211 - protein, 13 aa, synthetic construct
WEWLELKLGQSMY

SEQ ID NO: 212 - protein, 13 aa, synthetic construct
WGVLQFKLGQPMY

SEQ ID NO: 213 - protein, 13 aa, synthetic construct
WGWIQLWRGQPMY

SEQ ID NO: 214 - protein, 13 aa, synthetic construct
WGWLKFKYGMPMY

SEQ ID NO: 215 - protein, 13 aa, synthetic construct
WGYLRLGYGQPMY

SEQ ID NO: 216 - protein, 13 aa, synthetic construct
WGYLTIKLGQPMY

SEQ ID NO: 217 - protein, 13 aa, synthetic construct
WHFLKLETGQPMY

SEQ ID NO: 218 - protein, 13 aa, synthetic construct
WHFLSLKLGQPMY

SEQ ID NO: 219 - protein, 13 aa, synthetic construct
WHGLALYSGQPMY

SEQ ID NO: 220 - protein, 13 aa, synthetic construct
WHLIQIKLGQPMY

SEQ ID NO: 221 - protein, 13 aa, synthetic construct
WHLVQFWPGQPMY

SEQ ID NO: 222 - protein, 13 aa, synthetic construct
WHLVSIKWGQPMY

SEQ ID NO: 223 - protein, 13 aa, synthetic construct
WHTLSLKWGQPMY

SEQ ID NO: 224 - protein, 13 aa, synthetic construct
WHVGKLKAGSPMY

SEQ ID NO: 225 - protein, 13 aa, synthetic construct
WHVLRIKYGQPMY

SEQ ID NO: 226 - protein, 13 aa, synthetic construct
WHWGELWRGAPMY

SEQ ID NO: 227 - protein, 13 aa, synthetic construct
WHWVQLNTGQPMY

SEQ ID NO: 228 - protein, 13 aa, synthetic construct
WHWVTFKPGQSMY

SEQ ID NO: 229 - protein, 13 aa, synthetic construct
WKFLQLKYGQPMY

SEQ ID NO: 230 - protein, 13 aa, synthetic construct
WKFVQLKYGQSMY

SEQ ID NO: 231 - protein, 13 aa, synthetic construct
WKLLQIKMGQPMY

SEQ ID NO: 232 - protein, 13 aa, synthetic construct
WKLVQFKPGQPMY

SEQ ID NO: 233 - protein, 13 aa, synthetic construct
WKWLALKIGQSMY

SEQ ID NO: 234 - protein, 13 aa, synthetic construct
WKWLQFNWGQGMY

SEQ ID NO: 235 - protein, 13 aa, synthetic construct
WKYLNLKPGQPMY

SEQ ID NO: 236 - protein, 13 aa, synthetic construct
WKYVQLKIGQPMY

SEQ ID NO: 237 - protein, 13 aa, synthetic construct
WQWVALNLGQPMY

SEQ ID NO: 238 - protein, 13 aa, synthetic construct
WQWVQIKPGQSMY

SEQ ID NO: 239 - protein, 13 aa, synthetic construct
WQYIQLKLGMPMY

SEQ ID NO: 240 - protein, 13 aa, synthetic construct
WRLIQIKYGQPMY

SEQ ID NO: 241 - protein, 13 aa, synthetic construct
WRLLQLHMGQSMY

SEQ ID NO: 242 - protein, 13 aa, synthetic construct
WRLLQLKWGQPMY

SEQ ID NO: 243 - protein, 13 aa, synthetic construct
WRSLELKIGQPMY

SEQ ID NO: 244 - protein, 13 aa, synthetic construct
WRVLQFKTGEPMY

SEQ ID NO: 245 - protein, 13 aa, synthetic construct
WRWVQFKIGQGMY

SEQ ID NO: 246 - protein, 13 aa, synthetic construct
WSFLKLWTGQPMY

SEQ ID NO: 247 - protein, 13 aa, synthetic construct
WSFLQFNPGQPMY

SEQ ID NO: 248 - protein, 13 aa, synthetic construct
WSFLRLKPGSPMY

SEQ ID NO: 249 - protein, 13 aa, synthetic construct
WSTLRFKVGQPMY

SEQ ID NO: 250 - protein, 13 aa, synthetic construct
WSWLELKLGQSMY

SEQ ID NO: 251 - protein, 13 aa, synthetic construct
WSWVELKPGMPMY

SEQ ID NO: 252 - protein, 13 aa, synthetic construct
WSWVQLARGMPMY

SEQ ID NO: 253 - protein, 13 aa, synthetic construct
WTDLQLFKGQPMY

SEQ ID NO: 254 - protein, 13 aa, synthetic construct
WTILQLKPGAPMY

SEQ ID NO: 255 - protein, 13 aa, synthetic construct
WTLIQLRLGQPMY

SEQ ID NO: 256 - protein, 13 aa, synthetic construct
WTLLQLWRGQPMY

SEQ ID NO: 257 - protein, 13 aa, synthetic construct
WTTLQLKPGSPMY

SEQ ID NO: 258 - protein, 13 aa, synthetic construct
WTWLKLSPGQGMY

SEQ ID NO: 259 - protein, 13 aa, synthetic construct
WTWLTFKPGMPMY

SEQ ID NO: 260 - protein, 13 aa, synthetic construct
WTWVQLKPGASMY

SEQ ID NO: 261 - protein, 30 aa, synthetic construct
KHWGRLRAGQPMYGGGGKHWGRLRAGQPMY

SEQ ID NO: 262 - protein, 25 aa, synthetic construct
KHWGRLRAGQPMYGGGGRRRRRRRR

SEQ ID NO: 263 - protein, 25 aa, synthetic construct
KHWGRLRAGQPMYGGGGKKKKKKKK

SEQ ID NO: 264 - protein, 30 aa, synthetic construct
KHWGRLRAGQPMYGGGGYMPQGARLRGWHK

SEQ ID NO: 265 - protein, 30 aa, synthetic construct
YMPQGARLRGWHKGGGGKHWGRLRAGQPMY

SEQ ID NO: 266 - protein, 30 aa, synthetic construct
KHFLRFKPGQPMYGSGSKHFLRFKPGQPMY

SEQ ID NO: 267 - protein, 30 aa, synthetic construct
KHILKFKPGQPMYGGGGKHILKFKPGQPMY

SEQ ID NO: 268 - protein, 30 aa, synthetic construct
KHILKFKPGQPMYGSGSKHILKFKPGQPMY

SEQ ID NO: 269 - protein, 30 aa, synthetic construct
KHILKFKPGQPMYGSGSKHYLRFKPGQPMY

SEQ ID NO: 270 - protein, 30 aa, synthetic construct
KHILKFKPGQPMYGSGSWTVLTLKRGQSMY

SEQ ID NO: 271 - protein, 30 aa, synthetic construct
KHVLRFKPGQPMYGSGSKHVLRFKPGQPMY

SEQ ID NO: 272 - protein, 30 aa, synthetic construct
KHWIKLKNGQPMYGSGSKHWIKLKNGQPMY

SEQ ID NO: 273 - protein, 30 aa, synthetic construct
KHWLKFKPGQPMYGSGSKHWLKFKPGQPMY

SEQ ID NO: 274 - protein, 30 aa, synthetic construct
KHWLKIKIGEPMYGSGSKHWLKIKIGEPMY

SEQ ID NO: 275 - protein, 30 aa, synthetic construct
KHWLRFKPGQPMYGSGSKHWLRFKPGQPMY

SEQ ID NO: 276 - protein, 30 aa, synthetic construct
KHWLRFRPGQPMYGSGSKHWLRFRPGQPMY

SEQ ID NO: 277 - protein, 30 aa, synthetic construct
KHWLRIKWGEPMYGSGSKHWLRIKWGEPMY

SEQ ID NO: 278 - protein, 30 aa, synthetic construct
KHYLKFKPGQPMYGSGSKHYLKFKPGQPMY

SEQ ID NO: 279 - protein, 30 aa, synthetic construct
KHYLRFKPGQPMYGGGGKHYLRFKPGQPMY

SEQ ID NO: 280 - protein, 30 aa, synthetic construct
KHYLRFKPGQPMYGSGSKHILKFKPGQPMY

SEQ ID NO: 281 - protein, 30 aa, synthetic construct
KHYLRFKPGQPMYGSGSKHYLRFKPGQPMY

SEQ ID NO: 282 - protein, 30 aa, synthetic construct
KHYLRFKPGQPMYGSGSWTVLTLKRGQSMY

SEQ ID NO: 283 - protein, 30 aa, synthetic construct
PHWISLKKGQPMYGSGSPHWISLKKGQPMY

SEQ ID NO: 284 - protein, 30 aa, synthetic construct
WTVLTLKRGQSMYGGGGWTVLTLKRGQSMY

SEQ ID NO: 285 - protein, 30 aa, synthetic construct
WTVLTLKRGQSMYGSGSKHILKFKPGQPMY

SEQ ID NO: 286 - protein, 30 aa, synthetic construct
WTVLTLKRGQSMYGSGSKHYLRFKPGQPMY

SEQ ID NO: 287 - protein, 30 aa, synthetic construct
WTVLTLKRGQSMYGSGSWTVLTLKRGQSMY

SEQ ID NO: 288 - protein, 30 aa, synthetic construct
YMPEGIKIKLWHKGSGSKHWLKIKIGEPMY

SEQ ID NO: 289 - protein, 30 aa, synthetic construct
YMPEGWKIRLWHKGSGSKHWLRIKWGEPMY

SEQ ID NO: 290 - protein, 30 aa, synthetic construct
YMPQGKKLSIWHPGSGSPHWISLKKGQPMY

SEQ ID NO: 291 - protein, 30 aa, synthetic construct
YMPQGNKLKIWHKGSGSKHWIKLKNGQPMY

SEQ ID NO: 292 - protein, 30 aa, synthetic construct
YMPQGPKFKLIHKGGGGKHILKFKPGQPMY

SEQ ID NO: 293 - protein, 30 aa, synthetic construct
YMPQGPKFKLIHKGSGSKHILKFKPGQPMY

SEQ ID NO: 294 - protein, 30 aa, synthetic construct
YMPQGPKFKLWHKGSGSKHWLKFKPGQPMY

SEQ ID NO: 295 - protein, 30 aa, synthetic construct
YMPQGPKFKLYHKGSGSKHYLKFKPGQPMY

SEQ ID NO: 296 - protein, 30 aa, synthetic construct
YMPQGPKFRLFHKGSGSKHFLRFKPGQPMY

SEQ ID NO: 297 - protein, 30 aa, synthetic construct
YMPQGPKFRLVHKGSGSKHVLRFKPGQPMY

SEQ ID NO: 298 - protein, 30 aa, synthetic construct
YMPQGPKFRLWHKGSGSKHWLRFKPGQPMY

SEQ ID NO: 299 - protein, 30 aa, synthetic construct
YMPQGPKFRLYHKGGGGKHYLRFKPGQPMY

SEQ ID NO: 300 - protein, 30 aa, synthetic construct
YMPQGPKFRLYHKGSGSKHYLRFKPGQPMY

SEQ ID NO: 301 - protein, 30 aa, synthetic construct
YMPQGPRFRLWHKGSGSKHWLRFRPGQPMY

SEQ ID NO: 302 - protein, 30 aa, synthetic construct
YMSQGRKLTLVTWGGGGWTVLTLKRGQSMY

SEQ ID NO: 303 - protein, 30 aa, synthetic construct
YMSQGRKLTLVTWGSGSWTVLTLKRGQSMY

SEQ ID NO: 304-325 - skipped (22 entries)

SEQ ID NO: 326 - protein, 30 aa, synthetic construct
KCWLELKTGMPMYGGGGKCWLELKTGMPMY

SEQ ID NO: 327 - protein, 30 aa, synthetic construct
KHFLRFKPGQPMYGGGGKHFLRFKPGQPMY

SEQ ID NO: 328 - protein, 28 aa, synthetic construct
KHILKFKPGQPMYGGGGGEKEKEKEKEK

SEQ ID NO: 329 - protein, 28 aa, synthetic construct
KHILKFKPGQPMYGGGGGRRRRRRRRRR

SEQ ID NO: 330 - protein, 30 aa, synthetic construct
KHILKFKPGQPMYGGGGKHYLRFKPGQPMY

SEQ ID NO: 331 - protein, 30 aa, synthetic construct
KHILKFKPGQPMYGGGGWTVLTLKRGQSMY

SEQ ID NO: 332 - protein, 30 aa, synthetic construct
KHVLRFKPGQPMYGGGGKHVLRFKPGQPMY

SEQ ID NO: 333 - protein, 30 aa, synthetic construct
KHWIKLKNGQPMYGGGGKHWIKLKNGQPMY

SEQ ID NO: 334 - protein, 30 aa, synthetic construct
KHWLKFKPGQPMYGGGGKHWLKFKPGQPMY

SEQ ID NO: 335 - protein, 30 aa, synthetic construct
KHWLKIKIGEPMYGGGGKHWLKIKIGEPMY

SEQ ID NO: 336 - protein, 30 aa, synthetic construct
KHWLRFKPGQPMYGGGGKHWLRFKPGQPMY

SEQ ID NO: 337 - protein, 30 aa, synthetic construct
KHWLRFRPGQPMYGGGGKHWLRFRPGQPMY

SEQ ID NO: 338 - protein, 30 aa, synthetic construct
KHWLRFWPGQPMYGGGGKHWLRFWPGQPMY

SEQ ID NO: 339 - protein, 30 aa, synthetic construct
KHWLRIKWGEPMYGGGGKHWLRIKWGEPMY

SEQ ID NO: 340 - protein, 30 aa, synthetic construct
KHYLKFKPGQPMYGGGGKHYLKFKPGQPMY

SEQ ID NO: 341 - protein, 28 aa, synthetic construct
KHYLRFKPGQPMYGGGGGEKEKEKEKEK

SEQ ID NO: 342 - protein, 28 aa, synthetic construct
KHYLRFKPGQPMYGGGGGRRRRRRRRRR

SEQ ID NO: 343 - protein, 30 aa, synthetic construct
KHYLRFKPGQPMYGGGGKHILKFKPGQPMY

SEQ ID NO: 344 - protein, 30 aa, synthetic construct
KHYLRFKPGQPMYGGGGWTVLTLKRGQSMY

SEQ ID NO: 345 - protein, 30 aa, synthetic construct
PHWISLKKGQPMYGGGGPHWISLKKGQPMY

SEQ ID NO: 346 - protein, 30 aa, synthetic construct
PRWLQIKKGQSMYGGGGPRWLQIKKGQSMY

SEQ ID NO: 347 - protein, 30 aa, synthetic construct
WHILRFKNGMPMYGGGGWHILRFKNGMPMY

SEQ ID NO: 348 - protein, 30 aa, synthetic construct
WHWVQFGPGSPMYGGGGWHWVQFGPGSPMY

SEQ ID NO: 349 - protein, 30 aa, synthetic construct
WQMLKLKYGQPMYGGGGWQMLKLKYGQPMY

SEQ ID NO: 350 - protein, 28 aa, synthetic construct
WTVLTLKRGQSMYGGGGGEKEKEKEKEK

SEQ ID NO: 351 - protein, 28 aa, synthetic construct
WTVLTLKRGQSMYGGGGGRRRRRRRRRR

SEQ ID NO: 352 - protein, 30 aa, synthetic construct
WTVLTLKRGQSMYGGGGKHILKFKPGQPMY

SEQ ID NO: 353 - protein, 30 aa, synthetic construct
WTVLTLKRGQSMYGGGGKHYLRFKPGQPMY

SEQ ID NO: 354 - protein, 30 aa, synthetic construct
YMPEGIKIKLWHKGGGGKHWLKIKIGEPMY

SEQ ID NO: 355 - protein, 30 aa, synthetic construct
YMPEGWKIRLWHKGGGGKHWLRIKWGEPMY

SEQ ID NO: 356 - protein, 30 aa, synthetic construct
YMPMGNKFRLIHWGGGGWHILRFKNGMPMY

SEQ ID NO: 357 - protein, 30 aa, synthetic construct
YMPMGTKLELWCKGGGGKCWLELKTGMPMY

SEQ ID NO: 358 - protein, 30 aa, synthetic construct; identical to SEQ ID NO: 265

SEQ ID NO: 359 - protein, 30 aa, synthetic construct
YMPQGKKLSIWHPGGGGPHWISLKKGQPMY

SEQ ID NO: 360 - protein, 30 aa, synthetic construct
YMPQGNKLKIWHKGGGGKHWIKLKNGQPMY

SEQ ID NO: 361 - protein, 30 aa, synthetic construct
YMPQGPKFKLWHKGGGGKHWLKFKPGQPMY

SEQ ID NO: 362 - protein, 30 aa, synthetic construct
YMPQGPKFKLYHKGGGGKHYLKFKPGQPMY

SEQ ID NO: 363 - protein, 30 aa, synthetic construct
YMPQGPKFRLFHKGGGGKHFLRFKPGQPMY

SEQ ID NO: 364 - protein, 30 aa, synthetic construct
YMPQGPKFRLVHKGGGGKHVLRFKPGQPMY

SEQ ID NO: 365 - protein, 30 aa, synthetic construct
YMPQGPKFRLWHKGGGGKHWLRFKPGQPMY

SEQ ID NO: 366 - protein, 30 aa, synthetic construct
YMPQGPRFRLWHKGGGGKHWLRFRPGQPMY

SEQ ID NO: 367 - protein, 30 aa, synthetic construct
YMPQGPWFRLWHKGGGGKHWLRFWPGQPMY

SEQ ID NO: 368 - protein, 30 aa, synthetic construct
YMPQGYKLKLMQWGGGGWQMLKLKYGQPMY

SEQ ID NO: 369 - protein, 30 aa, synthetic construct
YMPSGPGFQVWHWGGGGWHWVQFGPGSPMY

SEQ ID NO: 370 - protein, 30 aa, synthetic construct
YMSQGKKIQLWRPGGGGPRWLQIKKGQSMY

SEQ ID NO: 371 - protein, 32 aa, synthetic construct
KHWGRLRAGQPMYEGEGGCKADGSWGVCCSGF

SEQ ID NO: 372 - protein, 32 aa, synthetic construct
YMPQGARLRGWHKEGEGGCKADGSWGVCCSGF

SEQ ID NO: 373 - protein, 32 aa, synthetic construct
FGSCCVGWSGDAKCGEGEGKHWGRLRAGQPMY

SEQ ID NO: 374 - protein, 32 aa, synthetic construct
FGSCCVGWSGDAKCGEGEGYMPQGARLRGWHK

SEQ ID NO: 375 - skipped

SEQ ID NO: 376 - protein, 4 aa, synthetic construct
GGGG

SEQ ID NO: 377 - protein, 4 aa, synthetic construct
GSGS

SEQ ID NO: 378 - protein, 6 aa, synthetic construct
GGTGGS

SEQ ID NO: 379 - protein, 15 aa, synthetic construct
GGGGSGGGGSGGGGS

SEQ ID NO: 380 - protein, 4 aa, synthetic construct
  VARIANT 3..4: note = Can be absent or present in repeats of any integer
EGEG

SEQ ID NO: 381 - protein, 4 aa, synthetic construct
  VARIANT 1..4: note = Can be present in repeats of any integer
GGGS

SEQ ID NO: 382 - protein, 4 aa, synthetic construct
  VARIANT 3..4: note = Can be absent or present in repeats of any integer
GSGS

SEQ ID NO: 383 - protein, 4 aa, synthetic construct
  VARIANT 3..4: note = Can be absent or present in repeats of any integer
KGKG

SEQ ID NO: 384 - protein, 4 aa, synthetic construct
  VARIANT 1..4: note = Can be present in repeats of any integer
KGEG

SEQ ID NO: 385 - protein, 9 aa, synthetic construct
RKKRRQRRR

SEQ ID NO: 386 - protein, 11 aa, synthetic construct
KKLFKKILKYL

SEQ ID NO: 387 - protein, 9 aa, synthetic construct
RRRRRRRRR

SEQ ID NO: 388 - protein, 18 aa, synthetic construct
KALKKLLAKWLAAAKALL

SEQ ID NO: 389 - protein, 14 aa, synthetic construct
LLLFLLKKRKKRKY

SEQ ID NO: 390 - protein, 23 aa, synthetic construct
SYFILRRRRKRFPYFFTDVRVAA

SEQ ID NO: 391 - protein, 13 aa, synthetic construct
KMDCRWRWKCCKK

SEQ ID NO: 392 - protein, 10 aa, synthetic construct
CRWRWKCCKK

SEQ ID NO: 393 - protein, 15 aa, synthetic construct
  VARIANT 3..4: note = Can be absent or present in repeats of any integer
FLGKKFKKYFLQLLK

SEQ ID NO: 394 - protein, 16 aa, synthetic construct
KTVLLRKLLKLLVRKI

SEQ ID NO: 395 - protein, 23 aa, synthetic construct
GIGKFLHSAKKWGKAFVGQIMNC

SEQ ID NO: 396 - protein, 8 aa, synthetic construct
RRRRRRRR

SEQ ID NO: 397 - protein, 9 aa, synthetic construct
RRRRRRRRR

SEQ ID NO: 398 - protein, 8 aa, synthetic construct
KKKKKKKK

SEQ ID NO: 399 - protein, 4 aa, synthetic construct
  VARIANT 1..2: note = Can be present in repeats of any integer
EGGC

SEQ ID NO: 400 - protein, 4 aa, synthetic construct
  VARIANT 3..4: note = Can be present in repeats of any integer
CGEG

SEQ ID NO: 401 - protein, 13 aa, synthetic construct
WHWLQLKPGQPMY

SEQ ID NO: 402 - protein, 13 aa, synthetic construct
KHILKFKPGQPMY

SEQ ID NO: 403 - protein, 13 aa, synthetic construct
KHYLRFKPGQPMY

SEQ ID NO: 404 - protein, 13 aa, synthetic construct
KHSLRLWPGQPMY

SEQ ID NO: 405 - protein, 13 aa, synthetic construct
KHWLTLFPGQPMY

SEQ ID NO: 406 - protein, 13 aa, synthetic construct
WQWLQLDRGQPMY

SEQ ID NO: 407 - protein, 13 aa, synthetic construct
WHSLQIKKGQPMY

SEQ ID NO: 408 - protein, 13 aa, synthetic construct
WHLLQFNWGQPMY

SEQ ID NO: 409 - protein, 13 aa, synthetic construct
WTVLTLKRGQSMY

SEQ ID NO: 410 - protein, 13 aa, synthetic construct
KHWLRFRPGQPMY

SEQ ID NO: 411 - protein, 13 aa, synthetic construct
KHWLRFWPGQPMY

SEQ ID NO: 412 - protein, 13 aa, synthetic construct
KHWLRIWPGQPMY

SEQ ID NO: 413 - protein, 13 aa, synthetic construct
KHWLRLWPGQPMY

SEQ ID NO: 414 - protein, 13 aa, synthetic construct
WHWLRLWPGQPMY

SEQ ID NO: 415 - protein, 26 aa, synthetic construct
WHWLQLKPGQPMYYMPQGPKLQLWHW

SEQ ID NO: 416 - protein, 26 aa, synthetic construct
YMPQGPKLQLWHWWHWLQLKPGQPMY

SEQ ID NO: 417 - protein, 30 aa, synthetic construct
WHWLQLKPGQPMYGGGGYMPQGPKLQLWHW

SEQ ID NO: 418 - protein, 30 aa, synthetic construct
YMPQGPKLQLWHWGGGGWHWLQLKPGQPMY

SEQ ID NO: 419 - protein, 21 aa, synthetic construct
RRRRRRRRWHWLQLKPGQPMY

SEQ ID NO: 420 - protein, 21 aa, synthetic construct
RRRRRRRRYMPQGPKLQLWHW

SEQ ID NO: 421 - protein, 5 aa, synthetic construct
GQPMY

SEQ ID NO: 422 - protein, 5 aa, synthetic construct
GQSMY

SEQ ID NO: 423 - protein, 9 aa, synthetic construct
RFKPGQPMY

(19) United States
(12) Patent Application Publication
(10) Pub. No.: US 2025/0382332 A1
(43) Pub. Date: Dec. 18, 2025

(54) NOVEL ALPHA-FACTOR BASED PEPTIDES WITH ANTIFUNGAL ACTIVITY

(71) Applicant: INVAIO SCIENCES, INC., Cambridge, MA (US)

(72) Inventors: Sowmya Ranganayaki RAMACHANDRAN, Bedford, MA (US); Gaurav KANDOI, Somerville, MA (US); Jahnavi Chandra PRASAD, Melrose, MA (US)

(21) Appl. No.: 19/236,447

(22) Filed: Jun. 12, 2025

Related U.S. Application Data
(60) Provisional application No. 63/659,289, filed on Jun. 12, 2024.

(51) Int. Cl. C07K 7/08; A01N 37/46; A01P 3/00; A61K 38/00; A61K 45/06; A61P 31/10; C07K 14/00; C07K 14/39

(52) U.S. Cl. CPC C07K 7/08; A01N 37/46; A01P 3/00; A61K 45/06; A61P 31/10; C07K 14/001; C07K 14/39; A61K 38/00; C07K 2319/10

(57) ABSTRACT

Disclosed herein are compositions for use in controlling fungal diseases, wherein the compositions comprise novel alpha-factor-derived anti-fungal peptides, or one or more polynucleotides encoding for the one or more antifungal peptides.

### CROSS REFERENCE TO RELATED APPLICATIONS

This application claims priority to provisional application U.S. Ser. No. 63/659,289, filed Jun. 12, 2024, which is incorporated herein by reference in its entirety.

### SEQUENCE LISTING XML

The instant application contains a sequence listing, which has been submitted in XML file format by electronic submission and is hereby incorporated by reference in its entirety. The XML file, created on Jun. 6, 2025, is named P14981US01.xml and is 358,396 bytes in size.

### FIELD

The present disclosure relates to antifungal peptides for controlling fungal diseases, compositions including the same, and methods related to producing and using the same. In particular, disclosed herein are compositions and method for controlling plant fungal diseases caused by Botrytis cinerea or mammalian fungal disease caused by Candida albicans, wherein the composition comprises one or more antifungal peptides.

### BACKGROUND

Fungi cause infections in both plants and mammals. Food rot and crop loss due to uncontrolled fungal pathogens of plants or plant products lead to significant agricultural and economic losses and are an increasing threat to global food production. Additionally, a wide variety of fungi, e.g., Candida species, cause opportunistic fungal infection in humans and animals.

Fungal persistence and antifungal resistance to commonly used antifungal agents are of high concern, both in the fields of agriculture and medicine. Thus, a need exists for compositions and methods for controlling or inhibiting the growth of fungal pathogens in a variety of biological systems.

### SUMMARY

Herein provided are novel alpha-factor variant peptides and methods for controlling fungal and/or bacterial diseases in plants or in mammals, e.g., plant fungal diseases caused by Botrytis cinerea, or mammalian fungal disease caused by Candida albicans, wherein the method comprises administering an effective amount of one or more alpha-factor variant peptides as disclosed herein.

Several embodiments related to an alpha-factor variant peptide comprising an amino acid sequence with at least 80%, at least 85%, at least 90%, at least 95%, at least 96%, at least 97%, at least 98%, or at least 99% identity to an amino acid sequence selected from SEQ ID NO: 1-260 and 401-414. Several embodiments relate to a peptide comprising an alpha-factor variant peptide comprising an amino acid sequence with at least 80%, at least 85%, at least 90%, at least 95%, at least 96%, at least 97%, at least 98%, or at least 99% identity to an amino acid sequence selected from SEQ ID NO: 1-260 and 401-414 linked directly or indirectly to a cell penetrating peptide. In some embodiments, the cell penetrating peptide is selected from Table 2.

Several embodiments relate to a polypeptide comprising two alpha-factor-variant peptide sequences linked directly or via a linker. Each alpha-factor peptide unit may be identical or different, in the forward or reverse orientation, and the linkage may be cleavable or non-cleavable, flexible or rigid, and may comprise sequences such as those listed in Table 1. In some embodiments, a polypeptide comprises a first alpha-factor variant peptide unit comprising an amino acid sequence with at least 80%, at least 85%, at least 90%, at least 95%, at least 96%, at least 97%, at least 98%, or at least 99% identity to an amino acid sequence selected from SEQ ID NO: 1-260 and 401-414 and a second alpha-factor variant peptide unit comprising an amino acid sequence with at least 80%, at least 85%, at least 90%, at least 95%, at least 96%, at least 97%, at least 98%, or at least 99% identity to an amino acid sequence selected from SEQ ID NO: 1-260 and 401-414. In some embodiments the polypeptide may comprise a linker sequence selected from Table 1 between the first and second alpha-factor variant peptide units, which may be in the same or opposite orientations. In some embodiments the polypeptide may further comprise one or more cell penetrating peptide units, e.g., one or more cell penetrating peptide units selected from Table 2. In some embodiments, the polypeptide comprises an amino acid sequence with at least 80%, at least 85%, at least 90%, at least 95%, at least 96%, at least 97%, at least 98%, or at least 99% identity to an amino acid sequence selected from SEQ ID NO: 261-374 and 413-420. In some embodiments, two alpha-factor variant peptide units are linked by a cleavable peptide linker, allowing for post-translational release of individual active alpha-factor variant peptides.

In some aspects, described herein is a composition comprising: an effective amount of one or more alpha-factor variant peptides, or one or more polynucleotides encoding the one or more alpha-factor variant peptides, wherein the one or more alpha-factor variant peptides has an amino acid sequence with at least 80% sequence identity to a peptide selected from the group consisting of SEQ ID NOs 1-303 and 326-374 and 401-420. In some embodiments, the one or more alpha-factor variant peptides is selected from the group consisting of SEQ ID NO: 9, SEQ ID NO: 12, SEQ ID NO: 13, SEQ ID NO: 18, SEQ ID NO: 52, SEQ ID NO: 62, SEQ ID NO: 68, SEQ ID NO: 72, SEQ ID NO: 261, SEQ ID NO:262, SEQ ID NO:263 and SEQ ID NO: 264.

In some embodiments, the composition comprises an effective amount of two or more alpha-factor variant peptides, and wherein the two or more alpha-factor variant peptides are linked with a linker. In some embodiments, the linker is selected from the group consisting of GG and SEQ ID NO: 376-384, 399-400. In some embodiments, the alpha-factor variant peptide further comprises at least one cell penetrating peptide (CPP), or a polynucleotide encoding for at least one CPP, wherein the CPP is selected from the group consisting of SEQ ID NO: 385-398. In some embodiments, the antimicrobial peptide and the CPP are fused.

In some embodiments, the composition further comprises an antifungal agent. In some embodiments, the antifungal mechanism of the antimicrobial peptide and the antifungal mechanism of the antifungal agent differ from each other. In some embodiments, the antifungal agent is selected from the group consisting of a FRAC group 3 fungicide, a FRAC group 48 fungicide, a FRAC group 7 fungicide, a FRAC group 9 fungicide, a FRAC group 11 fungicide, a FRAC group 12 fungicide, a fungicide that interacts with the fungal cell wall, a fungicide that inhibits beta-glucan synthesis, a fungicide that inhibits signal transduction, a fungicide that inhibits amino acid or protein synthesis, and a macrolide fungicide. In some embodiments, the macrolide fungicide is amphotericin B. In some embodiments, each of the one or more antifungal peptides is present at a concentration of between 1-200 μM. In some embodiments, each of the one or more antifungal peptides is present at a concentration of about 10 UM, about 15 μM, about 20 μM, about 50 UM or about 100 μM. In some embodiments, the composition further comprises a pharmaceutically acceptable carrier or an agriculturally acceptable carrier.

In some aspects, described herein is a method of decreasing growth or reproduction of a fungus, the method comprising providing a fungus with the antifungal composition comprising: an effective amount of one or more antifungal peptides, or one or more polynucleotides encoding for the one or more antifungal peptides operably linked to a heterologous promotor, wherein the one or more antifungal peptides has an amino acid sequence with at least 80% sequence identity to a peptide selected from the group consisting of SEQ ID NOs 1-303 and 326-374 and 401-420, thereby decreasing the growth or reproduction of the fungus. In some embodiments of the method, the one or more antifungal peptides is selected from the group consisting of SEQ ID NO: 9, SEQ ID NO: 12, SEQ ID NO: 13, SEQ ID NO: 18, SEQ ID NO: 52, SEQ ID NO: 62, SEQ ID NO: 68, SEQ ID NO: 72, SEQ ID NO: 261, SEQ ID NO:262, SEQ ID NO: 263 and SEQ ID NO:264.

In some embodiments of the method, the composition comprises an effective amount of two or more antifungal peptides, and wherein the two or more antifungal peptides are linked with a linker. In some embodiments of the method, the linker is selected from the group consisting of GG and SEQ ID NO: 376-384, 399-400. In some embodiments of the method, the composition further comprises at least one cell penetrating peptide (CPP), or a polynucleotide encoding for at least one CPP, wherein the CPP is selected from the group consisting of SEQ ID NO: 385-398. In some embodiments of the method, the antimicrobial peptide and the CPP are fused.

In some embodiments of the method, the composition further comprises an antifungal agent. In some embodiments of the method, the antifungal mechanism of the antimicrobial peptide and the antifungal mechanism of the antifungal agent differ from each other. In some embodiments of the method, the antifungal agent is selected from the group consisting of a FRAC group 3 fungicide, a FRAC group 48 fungicide, a FRAC group 7 fungicide, a FRAC group 9 fungicide, a FRAC group 11 fungicide, a FRAC group 12 fungicide, a fungicide that interacts with the fungal cell wall, a fungicide that inhibits beta-glucan synthesis, a fungicide that inhibits signal transduction, a fungicide that inhibits amino acid or protein synthesis, and a macrolide fungicide. In some embodiments of the method, the macrolide fungicide is amphotericin B.

In some embodiments of the method, each of the one or more antifungal peptides is present at a concentration of between 1-200 μM. In some embodiments of the method, each of the one or more antifungal peptides is present at a concentration of about 10 μM, about 15 UM, about 20 μM, about 50 UM or about 100 μM. In some embodiments of the method, the composition further comprises a pharmaceutically acceptable carrier or an agriculturally acceptable carrier.

In some embodiments of the method, the composition is provided to the fungus by directly contacting the fungus with the composition, or by delivering the composition to the environment of the fungus. In some embodiments of the method, the polynucleotide encoding one or more antifungal peptides is expressed in a fungus or in a plant. In some embodiments of the method, the fungus is a plant pathogen, a human pathogen, or an animal pathogen. In some embodiments of the method, the fungus is at least one selected from the group consisting of Botrytis sp., Fusarium sp., Phytophthora sp., Zymoseptoria sp., Aspergillus sp., Magnaporthe sp., Puccinia sp., Blumeria sp., Mycosphaerella sp., Colletotrichum sp., Ustilago sp., Melampsora sp., Phakopsora sp., Rhizoctonia sp., Aspergillus sp., Candida sp., Coccidioides sp., Histoplasma sp., Cryptococcus sp., Pneumocystis sp., and Blastomyces sp. In some embodiments of the method, the fungus is Botrytis cinerea, Fusarium graminaerum, Fusarium oxysporum, Zymoseptoria tritici, Pseudoperonospora cubensis, Aspergillus fumigatus, or Candida albicans.

In some aspects, described herein is a method of reducing the dose of an antifungal agent used for treatment of an infection caused by a fungus in a subject, the method comprising administering to the subject a composition comprising an antifungal agent and one or more antifungal peptides selected from the group consisting of SEQ ID NO: 1-303 and 326-374 and 401-420. In some embodiments of the method, the one or more antifungal peptides is selected from the group consisting of SEQ ID NO: 9, SEQ ID NO: 12, SEQ ID NO: 13, SEQ ID NO: 18, SEQ ID NO: 52, SEQ ID NO: 62, SEQ ID NO: 68, SEQ ID NO: 72, SEQ ID NO: 261, SEQ ID NO:262, SEQ ID NO:263 and SEQ ID NO:264. In some embodiments of the method, the antifungal agent is selected from the group consisting of a FRAC group 3, fungicide, a FRAC group 48 fungicide, a FRAC group 7 fungicide, a FRAC group 9 fungicide, a FRAC group 11 fungicide, a FRAC group 12 fungicide, a fungicide that interacts with the fungal cell wall, a fungicide that inhibits beta-glucan synthesis, a fungicide that inhibits signal transduction, a fungicide that inhibits amino acid or protein synthesis, and a macrolide fungicide. In some embodiments of the method, the macrolide fungicide is amphotericin B, and the fungus is C. albicans.

In some embodiments of the method, each of the one or more antifungal peptides is present at a concentration of between 1-200 μM. In some embodiments of the method, each of the one or more antifungal peptides is present at a concentration of about 10 μM, about 15 μM, about 20 μM, about 50 μM or about 100 μM. In some embodiments of the method, the composition further comprises a pharmaceutically acceptable carrier or an agriculturally acceptable carrier.

In some embodiments of the method, the subject is a plant, a human or an animal. In some embodiments of the method, the subject is a plant, and wherein the composition is administered by foliar application. In some embodiments of the method, the subject is a human or an animal, and the composition is provided topically or orally.

In some aspects, described herein is a method of decreasing germ tube formation by a fungus, comprising providing a fungus with the antifungal composition comprising: an effective amount of one or more antifungal peptides, or one or more polynucleotides encoding for the one or more antifungal peptides operably linked to a heterologous promotor, wherein the one or more antifungal peptides has an amino acid sequence with at least 80% sequence identity to a peptide selected from the group consisting of SEQ ID NOs 1-303 and 326-374 and 401-420, whereby the germ tube formation by the fungus is decreased, relative to a control fungus not provided with the antifungal composition.

In some aspects, described herein is a method of preventing or reducing disease caused by a fungal pathogen of a plant, comprising providing to a plant the antifungal composition providing a fungus with the antifungal composition comprising: an effective amount of one or more antifungal peptides, or one or more polynucleotides encoding for the one or more antifungal peptides operably linked to a heterologous promotor, wherein the one or more antifungal peptides has an amino acid sequence with at least 80% sequence identity to a peptide selected from the group consisting of SEQ ID NOs 1-303 and 326-374 and 401-420, whereby disease caused by the fungal pathogen is prevented or decreased in the plant, relative to a control plant not provided with the antifungal composition. In some aspects, described herein is a method of treating a subject with or at risk of a disease caused by a fungus, comprising administering to the subject the antifungal composition providing a fungus with the antifungal composition comprising: an effective amount of one or more antifungal peptides, or one or more polynucleotides encoding for the one or more antifungal peptides operably linked to a heterologous promotor, wherein the one or more antifungal peptides has an amino acid sequence with at least 80% sequence identity to a peptide selected from the group consisting of SEQ ID NOs 1-303 and 326-374 and 401-420, whereby the fungal disease is prevented or decreased in the subject, relative to a control subject not provided with the antifungal composition. In some embodiments of the method, the subject is: a) an animal selected from the group consisting of an invertebrate, an amphibian, a reptile, a bird, a cartilaginous or bony fish, and a non-human mammal; or b) a human.

In some aspects, described herein is a kit comprising: an effective amount of one or more antimicrobial peptides, or one or more polynucleotides encoding for the one or more antimicrobial peptides, wherein the one or more antimicrobial peptides has an amino acid sequence with at least 80% sequence identity to a peptide selected from the group consisting of SEQ ID NOs 1-303 and 326-374 and 401-420.

### DETAILED DESCRIPTION

The present invention includes a variety of aspects, which may be combined in different ways. The following descriptions are provided to list elements and describe some of the embodiments of the present invention. These elements are listed with initial embodiments, however it should be understood that they may be combined in any manner and in any number to create additional embodiments. The variously described examples and preferred embodiments should not be construed to limit the present invention to only the explicitly described systems, techniques, and applications. Further, this description should be understood to support and encompass descriptions and claims of all the various embodiments, systems, techniques, methods, devices, and applications with any number of the disclosed elements, with each element alone, and also with any and all various permutations and combinations of all elements in this or any subsequent application.

### Definitions

The term “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the term “amino acid” refers to an organic compound that contains amino (—NH3) and carboxylate (—CO2) functional groups, along with a side chain (R group) specific to each amino acid. Amino acid residues in polypeptides are in certain instance referred to herein by one letter amino acid codes as follows: G-Glycine (Gly); P-Proline (Pro); A-Alanine (Ala); V-Valine (Val); L-Leucine (Leu); I-Isoleucine (lie); M-Methionine (Met); C-Cysteine (Cys); F-Phenylalanine (Phe); Y-Tyrosine (Tyr); W-Tryptophan (Trp); H-Histidine (His); K-Lysine (Lys); R-Arginine (Arg); Q-Glutamine (Gin); N-Asparagine (Asn); E-Glutamic Acid (Glu); D-Aspartic Acid (Asp); S-Serine (Ser); or T-Threonine (Thr). As used herein, the terms “acidic” or “anionic” are used interchangeably to refer to amino acids, e.g., aspartic acid and glutamic acid.

As used herein, the terms “basic” and “cationic” are used interchangeably to refer to amino acids such as arginine, histidine, and lysine. The term “peptide”, “polypeptide” or “protein” as used herein, refers to any of various amides that are derived from two or more amino acids by combination of the amino group of one acid with the carboxyl group of another and are usually obtained by partial hydrolysis of proteins. The terms “peptide”, “polypeptide” or “protein” also include protein fragments, epitopes, catalytic sites, signaling sites, localization sites and the like. A peptide may further be a fusion or chimera peptide, which a used herein means a peptide having at least a first and second domain or moiety.

The term, “antimicrobial peptide,” as used herein refers to any peptide that has microbiocidal and/or microbiostatic activity, e.g., microbiocidal and/or microbiostatic activity towards gram-positive bacteria, gram-negative bacteria, or fungi. In some embodiments, antimicrobial peptides exhibit any one or more of the following characteristics of inhibiting the growth of microbial cells, killing microbial cells, disrupting or retarding stages of the microbial life cycle such as spore germination, sporulation, or mating, and/or disrupting microbial cell infection, penetration or spread within a plant or other susceptible subject, including a human, livestock, poultry, fish, or a companion animal (e.g., dog or cat). In some embodiments the antimicrobial peptide inhibits germ tube formation in fungi.

As used herein, the term “antimicrobial peptide precursor” refers to an antimicrobial peptide that comprises one or more domains that are cleaved-off post-translation of the peptide, e.g., a signal peptide sequence. In some embodiments, a precursor peptide comprises one or more domains that are cleaved off by proteases inside the cell, or by proteases outside the cell, yielding the mature form of the antimicrobial peptide. As used herein the term “antimicrobial peptide fragment” refers to a portion of the antimicrobial peptide, e.g., a peptide spanning part of the full-length antimicrobial peptide sequence.

As used herein, the terms “correspond,” “corresponding,” and the like, when used in the context of an amino acid position, mutation, and/or substitution in any given peptide with respect to a reference peptide sequence all refer to the amino acid residue in the given peptide sequence that has the same location in the given peptide as the residue in the reference amino acid sequence when the given peptide is aligned to the reference sequence. In certain embodiments, the alignment is an alignment of e.g., conserved cysteine residues in peptide and a reference peptide sequence.

As used herein, the terms “include,” “includes,” and “including” are to be construed as at least having the features to which they refer while not excluding any additional unspecified features.

Where a term is provided in the singular, other embodiments described by the plural of that term are also provided.

As used herein, a compound is referred to as “isolated” when it has been separated from at least one component with which it is naturally associated. For example, a polypeptide, e.g., an antimicrobial peptide, can be considered isolated if it is separated from contaminants including other polypeptides, polynucleotides and other metabolites. Isolated polypeptides can be either prepared synthetically, be purified from their natural environment, or be purified from cells expressing. Standard quantification methodologies known in the art can be employed to obtain and isolate the molecules of the invention.

The term “expression,” as used herein, or “expression” of a coding sequence (for example, a gene or a transgene) refers to the process by which the coded information of a nucleic acid transcriptional unit (including, e.g., genomic DNA or cDNA) is converted into an operational, non-operational, or structural part of a cell, often including the synthesis of a protein. Gene expression can be influenced by external signals; for example, exposure of a cell, tissue, or organism to an agent that increases or decreases gene expression. Expression of a gene can also be regulated anywhere in the pathway from DNA to RNA to protein. Regulation of gene expression occurs, for example, through controls acting on transcription, translation, RNA transport and processing, degradation of intermediary molecules such as mRNA, or through activation, inactivation, compartmentalization, or degradation of specific protein molecules after they have been made, or by combinations thereof. Gene expression can be measured at the RNA level or the protein level by any method known in the art, including, without limitation, Northern blot, RT-PCR, Western blot, or in vitro, in situ, or in vivo protein activity assay(s).

The term “nucleic acid” or “nucleic acid molecules” include single- and double-stranded forms of DNA; single-stranded forms of RNA; and double-stranded forms of RNA (dsRNA). The term “nucleotide sequence” or “nucleic acid sequence” refers to both the sense and antisense strands of a nucleic acid as either individual single strands or in the duplex. The term “gene” or “sequence” refers to a coding region operably joined to appropriate regulatory sequences capable of regulating the expression of the gene product (e.g., a polypeptide or a functional RNA) in some manner. A gene includes untranslated regulatory regions of DNA (e.g., promoters, enhancers, repressors, etc.) preceding (up-stream) and following (down-stream) the coding region (open reading frame, ORF) as well as, where applicable, intervening sequences (e.g., introns) between individual coding regions (e.g., exons).

A nucleic acid molecule may include either or both naturally occurring and modified nucleotides linked together by naturally occurring and/or non-naturally occurring nucleotide linkages. Nucleic acid molecules may be modified chemically or biochemically, or may contain non-natural or derivatized nucleotide bases, as will be readily appreciated by those of skill in the art. Such modifications include, for example, labels, methylation, substitution of one or more of the naturally occurring nucleotides with an analog, intemucleotide modifications (e.g., uncharged linkages: for example, methyl phosphonates, phosphotriesters, phosphoramidates, carbamates, etc.; charged linkages: for example, phosphorothioates, phosphorodithioates, etc.; pendent moieties: for example, peptides; intercalators: for example, acridine, psoralen, etc.; chelators; alkylators; and modified linkages: for example, alpha anomeric nucleic acids, etc.). The term “nucleic acid molecule” also includes any topological conformation, including single-stranded, double-stranded, partially duplexed, triplexed, hairpinned, circular, and padlocked conformations.

As described herein, “viroids” are small circular single stranded RNAs (ssRNAs) with no protein coating, characterized by secondary structures comprising intramolecular base pairing regions (stems) and unpaired loops and projections. Viroids are capable of invading and replicating in plants and may be virulent, mildly to moderately pathogenic or symbiotic with the host plant.

As used herein, the term “internal ribosome entry site” or “IRES” refers to a sequence (e.g., an RNA sequence) that is capable of recruiting ribosomes and translation machinery to initiate translation from the RNA sequence. IRES elements are typically between 100 and 800 nucleotides. In embodiments, the efficiency or effectiveness of an IRES in the compositions and methods described herein is tested, for example, by introducing the IRES into a circular RNA expression vector and determining the expression level of a downstream cistron protein, such as firefly luciferase, using an enzymatic reaction, or fluorescent readout using a reporter gene, such as Green Fluorescent Protein (GFP). Suitable IRES may be obtained from plant and plant viral IRES sequences, such as the encephalomyocarditis virus IRES (ECMV), maize hsp101 IRES 5′UTR, the tobacco virus crTMV CR-CP 148 IRES, tobacco Etch Virus (TEV) IRES 5′ UTR, and the hibiscus chlorosis virus (HCRSV) IRES. Furthermore, in embodiments, the IRES sequences are derived from non-plant eukaryotic viral sequences, including, but not limited to: acute Bee Paralysis Virus (ABPV), swine fever virus (CSFV), coxsackie virus B3 virus (CVB 3), encephalomyocarditis virus (ECMV), enterovirus 71 (E71), hepatitis A Virus (HAV), human rhinovirus (HRV 2), human lymphotropic virus (HTLV), and Polyoma Virus (PV).

As used herein, the term “effector” refers to a moiety that can be integrated into a recombinant polynucleotide (e.g., a viroid-derived vector) and is capable of modulating (e.g., modifying) the following states: a plant or plant cell; arthropod or arthropod cell; mollusc or mollusc cells; fungi or fungal cells; or nematodes or nematode cells. In embodiments, the effector comprises or is encoded by an RNA sequence, e.g., a single-stranded RNA (ssRNA) sequence. In embodiments, the effector comprises a coding sequence (e.g., a protein coding sequence). In embodiments, the effector is, for example, a regulatory RNA (e.g., lncRNA, circRNA, tRF, tRNA, rRNA, snRNA, snoRNA, or piRNA), an interfering RNA, dsRNA, microrna (miRNA), or precursor miRNA, phasiRNA, hcsiRNA, natsiRNA, or a guide RNA. In embodiments, the effector binds to a factor in the target host cell, e.g., binds to a nucleic acid, protein, peptide, DNA, RNA, or small molecule (e.g., metabolite or ion).

As used herein, when used in reference to a second element to describe the first element, the term “heterologous” means that the first element and the second element do not exist in nature in the arrangement as described. For example, a heterologous nucleic acid molecule or sequence is a nucleic acid molecule or sequence that: (a) is not native to the cell in which it is expressed, (b) is linked or fused to a nucleic acid molecule or sequence which is not linked or fused thereto in nature or which is not linked or fused thereto in the same manner as in nature, (c) has been artificially altered or mutated with respect to its natural state, or (d) expression is altered compared to its natural expression level under similar conditions. For example, a heterologous RNA relative to a viroid RNA means that the heterologous RNA is not present as part of or is associated with the viroid RNA in its naturally occurring state. For example, a recombinant polynucleotide such as provided by the present disclosure may include genetic sequences of two or more different classes of viruses that are “heterologous” in that they do not naturally occur together. In some embodiments, “heterologous” refers to a molecule; for example, cargo or payload (e.g., nucleic acids such as protein-encoding RNAs, ssRNAs, regulatory RNAs, interfering RNAs or guide RNAs) or structure (e.g., plasmid or gene editing systems) that does not occur naturally in plant viroids.

As used herein, the phrase “consensus sequence” refers to an amino acid, DNA or RNA sequence created by aligning two or more homologous sequences and deriving a new sequence having either the conserved or set of alternative amino acid, deoxyribonucleic acid, or ribonucleic acid residues of the homologous sequences at each position in the created sequence.

The phrases “percent identity” or “sequence identity” as used herein refer to the number of elements (e.g., amino acids or nucleotides) in a sequence that are identical within a defined length of two DNA, RNA segments in an alignment resulting in the maximal number of identical elements, and is calculated by dividing the number of identical elements by the total number of elements in the defined length of the aligned segments and multiplying by 100. Polynucleotide or polypeptide sequences may have substantial identity, substantial homology, or substantial complementarity to the selected region of the target gene, or target protein, respectively. As used herein “substantial identity” and “substantial homology” indicate sequences that have sequence identity or homology to each other. Generally, sequences that are substantially identical or substantially homologous will have about 75%, 80%, 85%, 90%, 91%, 92%, 93%, 94%, 95%, 96%, 97%, 98%, 99%, or 100% sequence identity.

Methods for aligning sequences for comparison are well-known in the art. A detailed consideration of sequence alignment methods and homology calculations can be found in, e.g., Altschul et al. (1990) J. Mol. Biol. 215:403-10. The National Center for Biotechnology Information (NCBI) Basic Local Alignment Search Tool (BLAST™; Altschul et al. (1990)) is available from several sources, including the National Center for Biotechnology Information (Bethesda, MD), and on the internet, for use in connection with several sequence analysis programs. A description of how to determine sequence identity using this program is available on the internet under the “help” section for BLAST™. For comparisons of nucleic acid sequences, the “Blast 2 sequences” function of the BLAST™ (Blastn) program may be employed using the default BLOSUM62 matrix set to default parameters. For alignment of polypeptide sequences, the Blastp program may be employed.

As used herein, the term “antibacterial agent” or “antibiotic” refers to a material that kills or inhibits the growth, proliferation, division, reproduction, or spread of bacteria, such as phytopathogenic bacteria, and includes bactericidal (e.g., disinfectant compounds, antiseptic compounds, or antibiotics) or bacteriostatic agents (e.g., compounds or antibiotics). Bactericidal antibiotics kill bacteria, while bacteriostatic antibiotics slow their growth or reproduction.

As used herein, the term “antifungal agent” or “antimycotic” refers to a material that kills or inhibits the growth, proliferation, division, reproduction, sporulation or germ tube formation in fungi, such as in plant pathogenic fungi (e.g., Botrytis or Fusarium spp.) or medically important fungi (e.g., Candida spp.). Antifungal agents include fungicidal compounds that kill the fungus, and fungistatic compounds that slow the growth or reproduction of the fungus.

As used herein, the term “antimicrobial agent” may refer to either an antibacterial or antifungal agent.

As used herein, “decreasing the growth or reproduction” of a microbial pathogen may refer to inhibiting the growth, proliferation, division, reproduction, spread, sporulation, or germ tube formation in one or more microbial pathogens. This may encompass “-cidal” effects, i.e., killing one or more microbial pathogens, or “static” effects, i.e., slowing the growth of one or more microbial pathogens.

As used herein, “effective amount” may refer to an amount sufficient to decrease the growth or reproduction of a microbial pathogen, or an amount sufficient to ameliorate symptoms caused by citrus greening disease. Such symptoms may include any one or more of the following: asymmetrical yellowing of veins and adjacent tissues; splotchy mottling of the entire leaf; premature defoliation; dieback of twigs; decay of feeder rootlets and lateral roots; decline in vigor; stunted growth, bear multiple off-season flowers; produce small, irregularly shaped fruit with a thick, pale peel that remains green at the bottom and tastes bitter.

As used herein, “cell penetrating peptide” refers to a peptide that can effectively traverse the components of a cell envelope (e.g., plasma membrane) and enter the cytoplasm of a cell.

### I. COMPOSITIONS

1. Antimicrobial peptides

Herein provided is a composition comprising at least one antimicrobial peptide, antimicrobial peptide precursor, antimicrobial peptide fragment, or antimicrobial peptide motif. In some embodiments the composition further comprises an antibacterial agent. In other embodiments, the composition further comprises a fungicidal agent. In some embodiments, the composition is formulated for application to a plant. In some embodiments, the composition is formulated for application to a mammal. In some embodiments, the antimicrobial peptide has microbiocidal and/or microbiostatic activity, e.g., microbiocidal and/or microbiostatic activity towards gram-positive bacteria, gram-negative bacteria, or fungi.

In some embodiments the composition comprises an effective amount of at least one antimicrobial peptide, antimicrobial peptide precursor, antimicrobial peptide fragment, or antimicrobial peptide motif, and that includes (a) an amino acid sequence of an antimicrobial peptide that has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity with a sequence selected from the group consisting of SEQ ID NO: 1-303 and 326-374 and 401-420; or (b) an amino acid sequence of an antimicrobial peptide motif; and a carrier. In some embodiments of this aspect, the antimicrobial peptide motif includes at least one of SEQ ID NO: 1-303 and 326-374 and 401-420. In additional embodiments of this aspect, which may be combined with any preceding embodiment, the antimicrobial peptide is active and/or toxic towards microbial cells. In some embodiments, the antimicrobial peptide is not toxic towards mammalian cells, e.g., human cells. In some embodiments, the amino acid sequence of the antimicrobial peptide variant, antimicrobial peptide precursor, or antimicrobial peptide fragment described herein is not one of the amino acid sequences disclosed in WO2023004435A1 or PCT/US2024/012652.

In some embodiments, an alpha-factor variant peptide as described herein includes one or more conservative amino acid substitutions relative to a reference alpha-factor peptide sequence, such as those represented by SEQ ID NOs: 1-303 or 326-374 or 401-420.

Conservative substitutions are those that preserve the physicochemical character of the residue, including polarity, charge, hydrophobicity, and steric bulk, and are expected to retain or enhance antifungal activity. Examples of conservative substitutions include: lysine (K) to arginine (R), or vice versa (basic residues); glutamic acid (E) to aspartic acid (D) (acidic residues); serine(S) to threonine (T) (polar uncharged); alanine (A) to valine (V), leucine (L) to isoleucine (I), or phenylalanine (F) to tyrosine (Y) (hydrophobic or aromatic residues). In some embodiments, such substitutions are introduced at non-critical positions as determined by activity assays or sequence alignments of naturally occurring alpha-factor homologs. In certain embodiments, peptides with up to 3, 5, or 7 conservative substitutions retain at least 90%, 80%, or 70% of the antifungal activity of the corresponding reference sequence. In some embodiments, positions amenable to conservative substitution include terminal residues, linker-proximal residues, or loop regions not directly involved in target membrane interaction.

Several embodiments relate to an alpha-factor variant peptide comprising a C-terminal motif of SEQ ID NO: 421 or 422, or a core motif SEQ ID NO:23, both of which are observed in multiple highly active alpha-factor variants, including SEQ ID NOs: 9, 12, and 13.

In some embodiments, a synthetic antimicrobial peptide or antimicrobial peptide precursor includes sequence derived from multiple naturally occurring antimicrobial peptides. In some embodiments, the antimicrobial peptides are derived from insects (e.g., scorpion peptides). In some embodiments, the antimicrobial peptides are derived from amphibians. In some embodiments, the antimicrobial polypeptide comprises more than one antimicrobial peptide derived from more than one source (e.g., a synthetic antimicrobial peptide that is a heterodimer or other multimer wherein the unit sequences are antimicrobial peptide sequences identified from different sources, optionally with a linker amino acid joining the unit sequences). Additionally, the antimicrobial peptide, antimicrobial peptide precursor, antimicrobial peptide fragment, or antimicrobial peptide motif may have additional amino acid residues added to it, for example, a linker sequence, a signal peptide sequence, a cell penetrating peptide, or similar. Exemplary linker sequences are provided in Table 1. Exemplary cell penetrating peptides are provided in Table 2.

TABLE 1
Exemplary linkers

| SEQ ID | Amino acid sequence |
|---|---|
| 376 | GGGG |
| 377 | GSGS |
| 378 | GGTGGS |
| 379 | GGGGSGGGGSGGGGS |
| 380 | (EG)n |
| 381 | (GGGS)n |
| 382 | (GS)n |
| 383 | (KG)n |
| 384 | (KGEG)n |
| 399 | (EG)nGC |
| 400 | CG(EG)n |

TABLE 2
Exemplary cell penetrating peptides

| SEQ ID | Amino acid sequence |
|---|---|
| 385 | RKKRRQRRR |
| 386 | KKLFKKILKYL |
| 387 | RRRRRRRRR |
| 388 | KALKKLLAKWLAAAKALL |
| 389 | LLLFLLKKRKKRKY |
| 390 | SYFILRRRRKRFPYFFTDVRVAA |
| 391 | KMDCRWRWKCCKK |
| 392 | CRWRWKCCKK |
| 393 | FLGKKFKKYFLQLLK |
| 394 | KTVLLRKLLKLLVRKI |
| 395 | GIGKFLHSAKKWGKAFVGQIMNC |
| 396 | RRRRRRRR |
| 397 | RRRRRRRRR |
| 398 | KKKKKKKK |

Whether alone or in combination with other active agents, the present antimicrobial peptides can be applied to plants or to subjects at a concentration in the range of from about 0.1 ng/ml to about 100 mg/ml, or from about 5 ng/ml to about 5 mg/ml, or from about 0.05 nM to about 50 mM, or from about 2.5 nM to about 50 mM. In some embodiments, the antimicrobial peptides are present at concentrations between 1-100 M. In some embodiments, the peptide concentration is about 10 μM. Peptides may be dissolved in a variety of solutions, at a pH in the range of from about 3.0 to about 9.0. Such compositions can be buffered using, for example, phosphate buffers between about 1 mM and 1 M, about 10 mM to about 100 mM, or about 15 mM to about 50 mM. In the case of low buffer concentrations, a salt can be added to increase the ionic strength. In certain embodiments, a sodium salt, including NaCl, in the range of from about 1 mM to about 1 M, about 1 mM, 5 mM, or 10 mM to about 20 mM, 50 mM, 100 mM, 150 mM, or 200 mM, or about 10 mM to about 100 mM, can be added or provided in compositions comprising antimicrobial peptides. In certain embodiments, a potassium salt, including KCl, in the range of about 1 mM, 5 mM, or 10 mM to about 20 mM, 50 mM, 100 mM, 150 mM, or 200 mM can be added or provided in compositions comprising defensin peptide variants and proteins. In certain embodiments, a calcium salt, including CaCl, in the range of about 0.1 mM, 0.5 mM, or 1 mM to about 2 mM, 5 mM, 10 mM, or 20 mM can be added or provided in compositions comprising microbial peptide variants.

In some embodiments the antimicrobial peptide inhibits germ tube formation in fungi. In some embodiments, the antimicrobial peptide is an antimicrobial peptide precursor. In some embodiments, the antimicrobial peptide precursor comprises domains that are cleaved-off post-translation of the peptide, e.g., a signal peptide sequence. In some embodiments, a precursor peptide comprises one or more domains that are cleaved off by proteases inside the cell, or by proteases outside the cell, yielding the mature form of the antimicrobial peptide. In some embodiments, the antimicrobial peptide precursor exhibits no antimicrobial activity. In some embodiments, the antimicrobial peptide is an antimicrobial peptide fragment, and refers to a portion of the antimicrobial peptide, e.g., a peptide spanning part of the full-length antimicrobial peptide sequence. In some embodiments, the antimicrobial peptide fragment is as active as the full-length antimicrobial peptide. In some embodiments, the antimicrobial peptide fragment is more active than the antimicrobial peptide from which it is derived.

In some embodiments, the antimicrobial peptide is a dimer or a chimer, e.g., wherein the antimicrobial peptide is linked to the same or to another antimicrobial peptide via a linker. An antimicrobial peptide provided herein can be operably linked to another antimicrobial peptide, antimicrobial peptide precursor, or antimicrobial peptide fragment via a spacer peptide sequence that is not susceptible to cleavage by an endoproteinase, including a plant endoproteinase. Such peptide linker sequences that join peptides in multimeric or multi-domain proteins have been disclosed (Argos, 1990; George R A, Heringa (2002)). Examples of suitable peptide sequences from multimeric or multi-domain proteins that can be used as spacer domains include immunoglobulin hinge regions from immunoglobulins, a linker between the lipoyl and E3 binding domain in pyruvate dehydrogenase (Turner et ah, 1993), a linker between the central and C-terminal domains in cysteine proteinase (P9; Mottram et ah, 1989), and functional variants thereof. Spacer peptides for use in the antimicrobial peptide variant proteins can also be wholly or partially synthetic peptide sequences. Such synthetic spacer peptides are designed to provide for a flexible linkage between at least one antimicrobial peptide variant and another peptide (including An antimicrobial peptide variant or antimicrobial peptide) and to be resistant to cleavage by endogenous plant or other endoproteinases. In certain embodiments, the length of the synthetic spacer peptide can be between about 3, 4, 8, 10, 12, or 16 and about 20, 24, 28, 30, 40, or 50 amino acid residues in length. In certain embodiments, the synthetic spacer peptide can comprise a glycine-rich or glycine/serine containing peptide sequence. The composition and design of peptides suitable for flexible linkage of protein domains described in the literature (Chen et al, 2013) can be adapted for use as spacer peptides in the antimicrobial peptide variant proteins provided herein. Spacer peptides useful for joining antimicrobial peptide monomers described in US Patent Appln. Publications US20190194268 and US20190185877, which are each incorporated herein by reference in their entireties, can also be used to join antimicrobial peptide variants disclosed herein to other antimicrobial peptide variants, antimicrobial peptide precursors, or antimicrobial peptide fragments.

An antimicrobial peptide variant provided herein can be operably linked to another antimicrobial peptide variant, antimicrobial peptide precursor, or antimicrobial peptide fragment via a linker peptide sequence that is susceptible to cleavage by an endoproteinase, including a plant endoproteinase. In certain embodiments, the resultant antimicrobial peptide variant protein can be expressed in a cell such that the endoproteinase cleaves the antimicrobial peptide variant protein to provide at least one antimicrobial peptide variant and another peptide (including an antimicrobial peptide variant or antimicrobial peptide). Such antimicrobial peptide variant proteins can be provided in a cellular compartment (e.g., cytoplasm, mitochondria, plastid, vacuole, or endoplasmic reticulum) or extracellular space (e.g., to the apoplast) having an endoproteinase that cleaves the linker peptide. Cleavable linker peptides are disclosed in WO2014078900, Vasivarama and Kirti, 2013a, Frangois et al, Vasivarama and Kirti, 2013b, and WO2017127558 can be used in the antimicrobial peptide variant proteins provided herein.

In some instances, the antimicrobial peptide is an antibody or antigen binding fragment thereof. For example, an agent described herein may be an antibody that blocks or potentiates activity and/or function of a component of the plant. The antibody may act as an antagonist or agonist of a polypeptide (e.g., enzyme or cell receptor) in the plant. The making and use of antibodies against a target antigen is known in the art. See, for example, Zhiqiang An (Ed.), Therapeutic Monoclonal Antibodies: From Bench to Clinic, 1st Edition, Wiley, 2009 and also Greenfield (Ed.), Antibodies: A Laboratory Manual, 2nd Edition, Cold Spring Harbor Laboratory Press, 2013, for methods of making recombinant antibodies, including antibody engineering, use of degenerate oligonucleotides, 5′-RACE, phage display, and mutagenesis; antibody testing and characterization; antibody pharmacokinetics and pharmacodynamics; antibody purification and storage; and screening and labeling techniques.

### 2. Polynucleotides Encoding Antimicrobial Peptides

In some embodiments, herein provided is a polynucleotide that comprises a DNA sequence encoding at least one antimicrobial peptide, antimicrobial peptide precursor, antimicrobial peptide fragment, or antimicrobial peptide motif, and that includes (a) an amino acid sequence of an antimicrobial peptide that has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity with a sequence selected from the group consisting of SEQ ID NO: 1-303 and 326-374 and 401-420; or (b) an amino acid sequence of an antimicrobial peptide motif; and a carrier. In some embodiments of this aspect, the antimicrobial peptide motif includes at least one of SEQ ID NO: 1-303 or 326-374 or 401-420. In additional embodiments of this aspect, which may be combined with any preceding embodiment, the antimicrobial peptide is active and/or toxic towards microbial cells. In some embodiments, the antimicrobial peptide is not toxic towards mammalian cells, e.g., human cells.

In some embodiments, the polynucleotide is a DNA, an RNA, or a plasmid. In some embodiments, the polynucleotide (e.g., DNA, RNA (e.g., mRNA, ASO, circular RNA, siRNA, shRNA, tRNA, dsRNA, or a combination thereof), or plasmid) encodes the antimicrobial peptide of any of the preceding embodiments. In some embodiments, the antimicrobial peptide is transcribed and/or translated in a cell. In some embodiments, the protein-encoding sequence is transcribed in a bacterial cell, e.g., in E. coli. In some embodiments, the protein-encoding sequence is transcribed in a plant cell. Depending on the cell type in which the antimicrobial peptide is expressed, the polynucleotide sequence encoding the antimicrobial peptide can be codon-optimized, using methodologies known in the art. In some embodiments a cell (e.g., an E. coli cell) is transformed with an expression cassette. Expression cassettes are DNA constructs wherein various promoter, coding (e.g., antimicrobial peptide variant encoding), and polyadenylation sequences are operably linked. In general, expression cassettes typically comprise a promoter that is operably linked to a sequence of interest, which is operably linked to a polyadenylation or terminator region. In certain instances, including the expression of recombinant or edited polynucleotides in monocot plants, it can also be useful to include an intron sequence. When an intron sequence is included, it is typically placed in the 5′ untranslated leader region of the recombinant or edited polynucleotide. In certain instances, it can also be useful to incorporate specific 5′ untranslated sequences in a recombinant or edited polynucleotide to enhance transcript stability or to promote efficient translation of the transcript. Expression cassettes and vectors for expression of other antimicrobial peptides or proteins in plants, including those disclosed in U.S. Pat. No. 10,253,328, which is incorporated herein by reference in its entirety, can be adapted for expression of the antimicrobial peptide variants in transgenic plants.

In some embodiments, the heterologous promoter is a bacterial promoter, a fungal promoter, an algal promoter, an animal promoter, or a plant promoter. In yet additional embodiments of this aspect, which may be combined with any preceding embodiment, the heterologous promoter is a plant expressible promoter, e.g., a promoter that is functional for driving expression in a plant cell. In some embodiments of this aspect, the plant expressible promoter is selected from the group of promoters of a ubiquitin promoter, a oestrum yellow virus promoter, a corn TrpA promoter, a OsMADS 6 promoter, a maize H3 histone promoter, a corn sucrose synthetase 1 promoter, a corn alcohol dehydrogenase 1 promoter, a corn heat shock protein promoter, a maize mtl promoter, a pea small subunit RuBP carboxylase promoter, a rice actin promoter, a rice cyclophilin promoter, a Ti plasmid mannopine synthase promoter, a Ti plasmid nopaline synthase promoter, a petunia chalcone isomerase promoter, a bean glycine rich protein 1 promoter, a potato patatin promoter, a lectin promoter, a CaMV 35S promoter, or a S-E9 small subunit RuBP carboxylase promoter. In some embodiments, the heterologous promoter is an inducible promoter, a tissue-specific promoter, a temporally specific promoter, or a developmentally specific promoter. Tissue-specific promoters are useful for limiting expression of the recombinant DNA construct and encoded antimicrobial peptide precursor, antimicrobial peptide fragment, or antimicrobial peptide motif to specific tissues (e.g., root, leaf, tuber, fruit, or seed) of a plant. In some embodiments, the heterologous promoter is a plant miRNA promoter, which can be inducible, tissue-specific, temporally specific, or developmentally specific; see, e.g., the tissue-specific promoters disclosed in U.S. Pat. No. 8,334,430 and the temporally specific promoters disclosed in U.S. Pat. No. 8,314,290. In some embodiments, the recombinant DNA construct includes further elements that are useful for expression control, such as expression-enhancing elements, transcript-stabilizing sequences, riboswitches, or recognition sites for miRNAs or siRNAs. For example, including a recognition site for a miRNA that is natively expressed in a specific tissue of a plant is expected to reduce or eliminate expression of the antimicrobial peptide precursor, antimicrobial peptide fragment, or antimicrobial peptide motif in that specific tissue. In additional embodiments of this aspect, the recombinant DNA construct further includes a nucleotide sequence encoding at least one secretion signal peptide functional in a cell. Still another aspect of the disclosure relates to a recombinant vector including the recombinant DNA construct of any one of the preceding embodiments. In some embodiments of this aspect, the vector includes a left T-DNA border and a right T-DNA border flanking the recombinant DNA construct. In some embodiments, the vector further comprises additional sequences flanking the recombinant DNA construct. The additional sequences may correspond to selectable markers, transposon ends, homologous arms, restriction sites, or other sequences suitable for downstream uses of the vector.

In additional embodiments of this aspect, the vector is a bacterial, viral, or viroid vector. Further provided are methods for modifying insects, mollusks, fungi, and nematodes by providing for consumption a plant comprising a recombinant polynucleotide e.g., recombinant ssRNAs, e.g., recombinant ssRNA vectors) comprising one or more sequences of or derived from a viroid and one or more heterologous effector sequences, e.g., an antimicrobial peptide, which have a biological effect on an organism. Methods for modifying plants by delivery of such recombinant viroid polynucleotides are described in WO2022020378A1, which is incorporated in its entirety herein.

### 3. Viroids

In some embodiments, recombinant polynucleotides are provided (e.g., recombinant ssRNAs, e.g., recombinant ssRNA vectors) comprising one or more sequences that are viroid or derived from viroid and one or more heterologous effector sequences that have a biological effect on an organism; compositions comprising such recombinant polynucleotides (e.g., compositions for topical application to plants); and methods of modifying plants by delivering such recombinant polynucleotides.

In some embodiments, the viroid recombinant polynucleotide encodes for at least one antimicrobial peptide, antimicrobial peptide precursor, antimicrobial peptide fragment, or antimicrobial peptide motif, and that includes (a) an amino acid sequence of an antimicrobial peptide that has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity with a sequence selected from the group consisting of SEQ ID NO: 1-303 and 326-374 and 401-420; or (b) an amino acid sequence of an antimicrobial peptide motif; and a carrier. In some embodiments of this aspect, the antimicrobial peptide motif includes at least one of SEQ ID NO: 1-303 or 326-374 or 401-420. In additional embodiments of this aspect, which may be combined with any preceding embodiment, the antimicrobial peptide is active and/or toxic towards microbial cells. In some embodiments, the antimicrobial peptide is not toxic towards mammalian cells, e.g., human cells.

Aspects of the disclosure relate to compositions comprising recombinant polynucleotides comprising: (i) A single stranded RNA (ssRNA) viroid sequence and (ii) a heterologous RNA sequence comprising or encoding an antimicrobial peptide. In some embodiments, the ssRNA viroid sequence is a viroid genome or derivative thereof. In some embodiments, the ssRNA viroid sequence is a viroid genome fragment or derivative thereof. Exemplary viroid sequences are described in WO2022020378A1, which is incorporated herein in its entirety.

### 4. Transgenic Cells and Plants

Another aspect of the disclosure relates to a transgenic cell comprising a polynucleotide, e.g., a recombinant vector of any one of the preceding claims. In further embodiments of this aspect, the cell is selected from a bacterial cell, a fungal cell, an algal cell, an animal cell, or a plant cell.

In some embodiments, the cell expresses an effective amount of at least one antimicrobial peptide, antimicrobial peptide precursor, antimicrobial peptide fragment, or antimicrobial peptide motif, and that includes (a) an amino acid sequence of an antimicrobial peptide that has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity with a sequence selected from the group consisting of SEQ ID NO: 1-303 and 326-374 and 401-420; or (b) an amino acid sequence of an antimicrobial peptide motif; and a carrier. In some embodiments of this aspect, the antimicrobial peptide motif includes at least one of SEQ ID NO: 1-303 or 326-374 or 401-420. In additional embodiments of this aspect, which may be combined with any preceding embodiment, the antimicrobial peptide is active and/or toxic towards microbial cells. In some embodiments, the antimicrobial peptide is not toxic towards mammalian cells, e.g., human cells.

In some embodiments, the cell comprising a recombinant vector expressing at least one antimicrobial peptide, antimicrobial peptide precursor, antimicrobial peptide fragment, or antimicrobial peptide motif further comprises one or more coating layers (e.g., chitosan and/or alginate coating layers) as described in WO2022076877A1, which is incorporated in its entirety herein.

In some embodiments, the cell that is transformed with a polynucleotide encoding at least one antimicrobial peptide of any of the preceding embodiments is selected from the group consisting of Escherichia coli, Bacillus subtilis, Corynebacterium glutamicum, Pichia pastoris, Saccharomyces cerevisiae, Kluyveromyces lactis, Kluyveromyces marxianus, Bacillus thuringiensis, Bacillus amyloliquefaciens, Pseudomonas putida, Pseudomonas entomophila, Pseudomonas chlororaphis, Pseudomonas fluorescens, Photorhabdus luminescens, Xenorhabdus nematophila, Lysinibacillus sphaericus, Kluyveromyces lactis, Streptomyces coelicolor, Streptomyces lividans, Streptomyces avermitilis, Streptomyces albus, and Entamoeba. In some embodiments, Escherichia coli, Bacillus subtilis, Corynebacterium glutamicum, or Pichia pastoris is transformed with a polynucleotide encoding at least one antimicrobial peptide of any of the preceding embodiments. In some embodiments, the transformed cell of any of the preceding embodiments is further coated with at least one biopolymer (e.g., an aliginate or a chitosan polymer or any combination of those two). In some embodiments the cell is a plant cell. In some embodiments, the plant cell is a dicot plant cell. In further embodiments of this aspect, the dicot plant cell is selected from the group of a soybean cell, a sunflower cell, a tomato cell, a potato cell, a Brassica spp. cell, a cotton cell, a sugar beet cell, or a tobacco cell. In additional embodiments of this aspect, the plant cell is a monocot plant cell. In further embodiments of this aspect, the monocot plant cell is selected from the group of a barley cell, a maize cell, an oat cell, a rice cell, a sorghum cell, a sugar cane cell, or a wheat cell. In yet another embodiment of this aspect, which may be combined with any of the preceding embodiments that has a transgenic cell, the antimicrobial peptide precursor, the antimicrobial peptide fragment, or the antimicrobial peptide motif is (a) transiently expressed, or (b) stably expressed.

In some embodiments, expression of the gene encoding the at least one antimicrobial peptide in the transformed cell (e.g., the transformed E. coli cell) is confirmed by the polymerase chain reaction (PCR) and sequencing of the PCR product, using methods common in the art.

Another aspect of the disclosure relates to a transgenic plant comprising a recombinant vector of any one of the preceding claims. In some embodiments, the transgenic plant comprises a vector expressing an effective amount of at least one antimicrobial peptide, antimicrobial peptide precursor, antimicrobial peptide fragment, or antimicrobial peptide motif, and that includes (a) an amino acid sequence of an antimicrobial peptide that has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity with a sequence selected from the group consisting of SEQ ID NO: 1-303 and 326-374 and 401-420; or (b) an amino acid sequence of an antimicrobial peptide motif; and a carrier. In some embodiments of this aspect, the antimicrobial peptide motif includes at least one of SEQ ID NO: 1-303 or 326-374 or 401-420. In additional embodiments of this aspect, which may be combined with any preceding embodiment, the antimicrobial peptide is active and/or toxic towards microbial cells.

In some embodiments, the antimicrobial peptide is not toxic towards mammalian cells, e.g., human cells. In some embodiments the transgenic plant is chimeric, having some cells that are transgenic (e.g., expressing a recombinant DNA construct as disclosed herein) and some cells that are not transgenic. Related embodiments include a grafted plant, wherein the rootstock is transgenic (e.g., expressing a recombinant DNA construct as disclosed herein) and the grafted scion is not transgenic; or wherein the rootstock is not transgenic, and the scion is transgenic. In embodiments, the modified genome is the nuclear genome of the plant; in other embodiments, the modified genome is the genome of the plant's chloroplasts or mitochondria.

In some embodiments of this aspect, the transgenic plant is a dicot plant. In further embodiments of this aspect, the dicot plant is selected from the group of a soybean plant, a sunflower plant, a tomato plant, a Brassica spp. plant, a cotton plant, a sugar beet plant, or a tobacco plant. In additional embodiments of this aspect, the transgenic plant is a monocot plant. In further embodiments of this aspect, the monocot plant is selected from the group of a barley plant, a maize plant, an oat plant, a rice plant, a sorghum plant, a sugar cane plant, or a wheat plant. In yet another embodiment of this aspect, the plant has improved resistance to the fungal pathogen, in comparison to a control plant that does not include the transgenic plant cell. In still another embodiment of this aspect, the fungal pathogen is one of an Aspergillus species; Magnaporthe oryzae, Botrytis cinerea, a Puccinia species.; Fusarium graminearum, Fusarium oxysporum, Blumeria graminis, Mycosphaerella graminicola, a Colletotrichum species, Ustilago maydis, Melampsora lini, Phakopsora pachyrhizi, or Rhizoctonia solani.

Plants and plant cells are of any species of interest, including dicots and monocots. Plants of interest include row crop plants, fruit-producing plants and trees, vegetables, trees, and ornamental plants including ornamental flowers, shrubs, trees, groundcovers, and turf grasses. Examples of commercially important cultivated crops, trees, and plants include: alfalfa (Medicago sativa), almonds (Prunus dulcis), apples (Malus x domestica), apricots (Prunus armeniaca, P. brigantine, P. mandshurica, P. mume, P. sibiricd), artichoke (Cynara cardunculus var. scolymus), asparagus (Asparagus officinalis), avocado (Persea americana), bananas (Musa spp.), barley (Hordeum vulgare), beans (Phaseolus spp.), blueberries and cranberries (Vaccinium spp.), Brazil nut (Bertholletia excelsa), cacao (Theobroma cacao), calamansi (Citrus x microcarpa), canola and rapeseed or oilseed rape, (Brassica napus), Polish canola (Brassica rapa), and related cruciferous vegetables including broccoli, kale, cabbage, and turnips (Brassica carinata, B. juncea, B. oleracea, B. napus, B. nigra, and B. rapa, and hybrids of these), carnation (Dianthus caryophyllus), carrots (Daucus carota sativus), cashew (Anacardium occidentale), cassava (Manihot esculentum), celery (Apium graveolens), cherry (Prunus avium), chestnut (Castanea spp.), chickpea or garbanzo (Cicer arietinum), chicory (Cichorium intybus), chili peppers and other Capsicum peppers (Capsicum annuum, C. frutescens, C. chinense, C. pubescens, C. baccatum), chrysanthemums (Chrysanthemum spp.), citron (Citrus medica), coconut (Cocos nucifera), coffee (wild and domesticated Coffea spp, including Coffea arabica, Coffea canephora, and Coffea liberica), cotton (Gossypium hirsutum L.), cowpea (Vigna unguiculata and other Vigna spp.), fava beans (Viciafaba), cucumber (Cucumis sativus), currants and gooseberries (Ribes spp.), date (Phoenix dactylifera), duckweeds (family Lemnoideae), eggplant or aubergine (Solanum melongena), elderberries (Sambucus spp.), eucalyptus (Eucalyptus spp.), flax (Linum usitatissumum L.), geraniums (Pelargonium spp.), ginger (Zingiber officinale), ginseng (Panax spp.), grapefruit (Citrus x paradisi), grapes (Vitis spp.) including wine grapes (Vitis vinifera and hybrids thereof), guava (Psidium guajava), hazelnut (Corylus avellana, Corylus spp.), hemp and cannabis (Cannabis sativa and Cannabis spp.), hops (Humulus lupulus), horseradish (Armoracia rusticana), irises (Iris spp.), jackfruit (Artocarpus heterophyllus), kiwifruits (Actinidia spp.), kumquat (Citrus japonica), lemon (Citrus limon), lentil (Lens culinaris), lettuce (Lactuca sativa), limes (Citrus spp.), lychee (Litchi chinensis), macadamias (Macadamia spp.), maize or corn (Lea mays L.), mandarin (Citrus reticulata), mango (Mangifera indicci), mangosteen (Garcinia mangostana), melon (Cucumis melo), millets (Setaria spp., Echinochloa spp., Eleusine spp., Panicum spp., Pennisetum spp.), oats (Avena sativa), oil palm (Ellis quineensis), okra (Abelmoschus esculentus), olive (Olea europaea), onion (Allium cepa) and other alliums (Allium spp.), orange (Citrus sinensis), papaya (Carica papaya), parsnip (Pastinaca sativa), passionfruit (Passiflora edulis), pecan (Carya illinoinensis), peaches and nectarines (Prunus persica), pear (Pyrus spp.), pea (Pisum sativum), peanut (Arachis hypogaea), peonies (Paeonia spp.), persimmons (Diospyros kaki, Diospyros spp.), petunias (Petunia spp.), pineapple (Ananas comosus), pistachio (Pistacia verci), plantains (Musa spp.), plum (Prunus domestica), poinsettia (Euphorbia pulcherrima), pomelo (Citrus maxima), poplar (Populus spp.), potato (Solanum tuberosum), pumpkins and squashes (Cucurbita pepo, C. maxima, C. moschata), quince (Cydonia oblonga), raspberries (Rubus idaeus, Rubus occidentalis, Rubus spp.), rhubarbs (Rheum spp.), rice (Oryza sativa L.), roses (Rosa spp.), rubber (Hevea brasiliensis), rye (Secale cereale), safflower (Carthamus tinctorius L.), satsuma (Citrus unshiu), sesame seed (Sesame indium), sorghum (Sorghum bicolor), sour orange (Citrus x aurantium), soursop (Annona muricata), soybean (Glycine max L.), strawberries (Fragaria spp., Fragaria x ananassa), sugar beet (Beta vulgaris), sugarcanes (Saccharum spp.), sunflower (Helianthus annuus), sweet potato (Ipomoea batatas), tamarind (Tamarindus indicci), tangerine (Citrus tangerina), tea (Camellia sinensis), tobacco (Nicotiana tabacum L.), tomatillo (Physalis philadelphica), tomato (Solanum lycopersicum or Lycopersicon esculentum), tulips (Tulipa spp.), walnuts (Juglans spp. L.), watermelon (Citrullus lanatus), wheat (Triticum aestivum), and yams (Discorea spp.). Wild relatives of domesticated plants are also of interest.

A further aspect of the disclosure relates to a transgenic seed of the transgenic plant of any of the preceding embodiments, wherein said seed includes the recombinant DNA construct of any of the preceding embodiments. An additional aspect of the disclosure relates to an FI progeny plant having at least one parent the transgenic plant of any of the preceding embodiments, wherein the FI progeny plant includes any of the recombinant DNA constructs of the preceding embodiments.

Yet another aspect of the disclosure relates to a harvested product produced from the transgenic plant of any of the preceding embodiments, wherein the harvested product includes the recombinant DNA construct. In some embodiments of this aspect, the harvested product is a fruit, a leaf, a stem, a flower, a root, a tuber, or a seed.

### 5. Compositions Comprising Combinations

Numerous conventional microbial antibiotics and chemical antimicrobial agents (e.g., fungicides) with which the present antimicrobial peptides can be combined are described in Worthington and Walker (1983) The Pesticide Manual, Seventh Edition, British Crop Protection Council. These include, for example, polyoxins, nikkomycins, carboxy amides, aromatic carbohydrates, carboxines, morpholines, inhibitors of sterol biosynthesis, and organophosphorous compounds. In addition, azoles, triazoles and echinocandins fungicides can also be used. Other active ingredients which can be formulated in combination with the present antimicrobial peptides and proteins include, for example, insecticides, attractants, sterilizing agents, acaricides, nematicides, and herbicides. U.S. Pat. No. 5,421,839, which is incorporated herein by reference in its entirety, contains a comprehensive summary of the many active agents with which substances such as the present antimicrobial defensin peptide variants and proteins can be formulated.

### a) Antimicrobial Peptides and Antibiotics

In some embodiments the antimicrobial peptide compositions described herein can further include an antibacterial agent, e.g., an antibiotic agent or a bactericide. In some instances, the compositions include two or more (e.g., 2, 3, 4, 5, 6, 7, 8, 9, 10, or more than 10) different antibacterial agents. In some embodiments, the presence of an antibacterial agent in the composition can further decrease the fitness of (e.g., decrease growth or kill) a bacterial plant pest (e.g., a bacterial plant pathogen). In some embodiments, the antimicrobial peptide and the antibiotic act cooperatively, e.g., the antimicrobial effect of the antimicrobial peptide and antibiotic is about equal to the sum of their individual effects. In some embodiments, the antimicrobial peptide and the antibiotic act synergistically, e.g., the antimicrobial effect of the antimicrobial peptide and antibiotic is greater than the sum of their individual effects.

In some embodiments the composition comprises at least one bactericide and an effective amount of at least one antimicrobial peptide, antimicrobial peptide precursor, antimicrobial peptide fragment, or antimicrobial peptide motif, and that includes (a) an amino acid sequence of an antimicrobial peptide that has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity with a sequence selected from the group consisting of SEQ ID NO: 1-303 and 326-374 and 401-420; or (b) an amino acid sequence of an antimicrobial peptide motif; and a carrier. In some embodiments of this aspect, the antimicrobial peptide motif includes at least one of SEQ ID NO: 1-303 or 326-374 or 401-420. In additional embodiments of this aspect, which may be combined with any preceding embodiment, the antimicrobial peptide is active and/or toxic towards microbial cells. In some embodiments, the antimicrobial peptide is not toxic towards mammalian cells, e.g., human cells.

A composition including a composition comprising an antimicrobial peptide and an antibiotic as described herein can be contacted with a target pest, or plant infested thereof, in an amount and for a time sufficient to: (a) reach a target level (e.g., a predetermined or threshold level) of antibiotic concentration inside or on the target pest; and (b) decrease fitness of the target pest. The antibiotics described herein may be formulated in a composition for any of the methods described herein, and in certain instances, may be associated with the antimicrobial peptides described herein.

The antibiotic described herein may target any bacterial function or growth processes and may be either bacteriostatic (e.g., slow or prevent bacterial growth) or bactericidal (e.g., kill bacteria). In some instances, the antibiotic is a bactericidal antibiotic. In some instances, the bactericidal antibiotic is one that targets the bacterial cell wall (e.g., penicillins and cephalosporins); one that targets the cell membrane (e.g., polymyxins); or one that inhibits essential bacterial enzymes (e.g., rifamycins, lipiarmycins, quinolones, and sulfonamides). In some instances, the bactericidal antibiotic is an aminoglycoside (e.g., kasugamycin). In some instances, the antibiotic is a bacteriostatic antibiotic. In some instances, the bacteriostatic antibiotic targets protein synthesis (e.g., macrolides, lincosamides, and tetracyclines). Additional classes of antibiotics that may be used herein include cyclic lipopeptides (such as daptomycin), glycylcyclines (such as tigecycline), oxazolidinones (such as linezolid), or lipiarmycins (such as fidaxomicin). Examples of antibiotics include rifampicin, ciprofloxacin, doxycycline, ampicillin, and polymyxin B. The antibiotic described herein may have any level of target specificity (e.g., narrow- or broad-spectrum). In some instances, the antibiotic is a narrow-spectrum antibiotic, and thus targets specific types of bacteria, such as gram-negative or gram-positive bacteria. Alternatively, the antibiotic may be a broad-spectrum antibiotic that targets a wide range of bacteria. In some embodiments, the antimicrobial peptide targets a different bacterial function or growth process than the antibiotic.

Examples of antibacterial agents suitable for the treatment of animals include Penicillins (Amoxicillin, Ampicillin, Bacampicillin, Carbenicillin, Cloxacillin, Dicloxacillin, Flucloxacillin, Mezlocillin, Nafcillin, Oxacillin, Penicillin G, Crysticillin 300 A. S., Pentids, Permapen, Pfizerpen, Pfizerpen-AS, Wycillin, Penicillin V, Piperacillin, Pivampicillin, Pivmecillinam, Ticarcillin), Cephalosporins (Cefacetrile (cephacetrile), Cefadroxil (cefadroxyl), Cefalexin (cephalexin), Cefaloglycin (cephaloglycin), Cefalonium (cephalonium), Cefaloridine (cephaloradine), Cefalotin (cephalothin), Cefapirin (cephapirin), Cefatrizine, Cefazaflur, Cefazedone, Cefazolin (cephazolin), Cefradine (cephradine), Cefroxadine, Ceftezole, Cefaclor, Cefamandole, Cefmetazole, Cefonicid, Cefotetan, Cefoxitin, Cefprozil (cefproxil), Cefuroxime, Cefuzonam, Cefcapene, Cefdaloxime, Cefdinir, Cefditoren, Cefetamet, Cefixime, Cefmenoxime, Cefodizime, Cefotaxime, Cefpimizole, Cefpodoxime, Cefteram, Ceftibuten, Ceftiofur, Ceftiolene, Ceftizoxime, Ceftriaxone, Cefoperazone, Ceftazidime, Cefclidine, Cefepime, Cefluprenam, Cefoselis, Cefozopran, Cefpirome, Cefquinome, Ceftobiprole, Ceftaroline, Cefaclomezine, Cefaloram, Cefaparole, Cefcanel, Cefedrolor, Cefempidone, Cefetrizole, Cefivitril, Cefmatilen, Cefmepidium, Cefovecin, Cefoxazole, Cefrotil, Cefsumide, Cefuracetime, Ceftioxide, Combinations, Ceftazidime/Avibactam, Ceftolozane/Tazobactam), Monobactams (Aztreonam), Carbapenems (Imipenem, Imipenem/cilastatin.Doripenem, Ertapenem, Meropenem, Meropenem/vaborbactam), Macrolide (Azithromycin, Erythromycin, Clarithromycin, Dirithromycin, Roxithromycin, Telithromycin), Lincosamides (Clindamycin, Lincomycin), Streptogramins (Pristinamycin, Quinupristin/dalfopristin), Aminoglycoside (Amikacin, Gentamicin, Kanamycin, Neomycin, Netilmicin, Paromomycin, Streptomycin, Tobramycin), Quinolone (Flumequine, Nalidixic acid, Oxolinic acid, Piromidic acid, Pipemidic acid, Rosoxacin, Second Generation, Ciprofloxacin, Enoxacin, Lomefloxacin, Nadifloxacin, Norfloxacin, Ofloxacin, Pefloxacin, Rufloxacin, Balofloxacin, Gatifloxacin, Grepafloxacin, Levofloxacin, Moxifloxacin, Pazufloxacin, Sparfloxacin, Temafloxacin, Tosufloxacin, Besifloxacin, Delafloxacin, Clinafloxacin, Gemifloxacin, Prulifloxacin, Sitafloxacin, Trovafloxacin), Sulfonamides (Sulfamethizole, Sulfamethoxazole, Sulfisoxazole, Trimethoprim-Sulfamethoxazole), Tetracycline (Demeclocycline, Doxycycline, Minocycline, Oxytetracycline, Tetracycline, Tigecycline), Other (Lipopeptides, Fluoroquinolone, Lipoglycopeptides, Cephalosporin, Macrocyclics, Chloramphenicol, Metronidazole, Tinidazole, Nitrofurantoin, Glycopeptides, Vancomycin, Teicoplanin, Lipoglycopeptides, Telavancin, Oxazolidinones, Linezolid, Cycloserine 2, Rifamycins, Rifampin, Rifabutin, Rifapentine, Rifalazil, Polypeptides, Bacitracin, Polymyxin B, Tuberactinomycins, Viomycin, Capreomycin).

In some embodiments, the composition comprises an antimicrobial peptide and antibiotic present in about an equal molar ratio, e.g., about 1:1. In some embodiments, the composition comprises an antimicrobial peptide and antibiotic present in an unequal molar ratio, e.g., about 1000:1, about 500:1, about 250:1, about 100:1, about 50:1, about 25:1, about 20:1, about 15:1, about 10:1, about 5:1, about 2:1, about 1:2, about 1:5, about 1:10, about 1:15, about 1:20, about 1:25, about 1:50, about 1:100, about 1:250, about 1:500, or about 1:1000.

In some embodiments, a composition comprises at least one antimicrobial peptide and oxytetracycline or a derivative thereof. In some embodiments the composition comprising at least one antimicrobial peptide and oxytetracycline inhibits the growth of CLas. In some embodiments, the composition comprising at least one antimicrobial peptide and oxytetracycline treats or prevents citrus greening (HLB). In some embodiments, the composition of any of the preceding embodiments comprises at least one antimicrobial peptide and an antibiotic that kills an obligate microbial symbiont in an insect pest. In some embodiments the insect pest is an aphid. In some embodiments the obligate microbial symbiont is Buchnéra sp.

One skilled in the art will appreciate that a suitable concentration of each antibiotic in the composition depends on factors such as efficacy, stability of the antibiotic, number of distinct antibiotics, the formulation, and methods of application of the composition.

### b) Antimicrobial Peptides and Antimycotics

In some embodiments the antimicrobial peptide compositions described herein can further include an antifungal agent, e.g., a fungicidal or a fungistatic agent. In some instances, the compositions include two or more (e.g., 2, 3, 4, 5, 6, 7, 8, 9, 10, or more than 10) different antifungal agents. In some embodiments, the presence of an antifungal agent in the composition can further decrease the fitness of (e.g., decrease growth or kill) a fungal plant pest (e.g., a fungal plant pathogen). In some embodiments, the antimicrobial peptide and the antifungal agent act cooperatively. In some embodiments, the antimicrobial peptide and the antifungal agent act synergistically.

In some embodiments the composition comprises at least one antifungal agent and an effective amount of at least one antimicrobial peptide, antimicrobial peptide precursor, antimicrobial peptide fragment, or antimicrobial peptide motif, and that includes (a) an amino acid sequence of an antimicrobial peptide that has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity with a sequence selected from the group consisting of SEQ ID NO: 1-303 and 326-374 and 401-420; or (b) an amino acid sequence of an antimicrobial peptide motif; and a carrier. In some embodiments of this aspect, the antimicrobial peptide motif includes at least one of SEQ ID NO: 1-303 or 326-374 or 401-420. In additional embodiments of this aspect, which may be combined with any preceding embodiment, the antimicrobial peptide is active and/or toxic towards microbial cells. In some embodiments, the antimicrobial peptide is not toxic towards mammalian cells, e.g., human cells.

A composition including a composition comprising an antimicrobial peptide and an antifungal agent as described herein can be contacted with a target pest, or plant infested thereof, in an amount and for a time sufficient to: (a) reach a target level (e.g., a predetermined or threshold level) of antifungal concentration inside or on the target pest; and (b) decrease fitness of the target pest. The antifungal agents described herein may be formulated in a composition for any of the methods described herein, and in certain instances, may be associated with the antimicrobial peptides described herein.

The disclosure provides a composition comprising at least one antimicrobial peptide, antimicrobial peptide precursor, antimicrobial peptide fragment, or antimicrobial peptide motif of any one of the preceding embodiments, and at least one fungicide of any one of the preceding embodiments. In some embodiments, the fungicide is classified as a FRAC group 3 fungicide, a polyene macrolide fungicide, a cell membrane-interacting antifungal peptides and/or antimicrobial peptide (AMP), or a cyclic lipopeptide fungicide. In some embodiments, fungicide inhibits sterol biosynthesis in fungal cell membranes, and is classified as a FRAC group 3 fungicide. In some embodiments, the FRAC group 3 fungicide is an azole or a nitrogen-containing heterocycle having a 5- to 6-membered ring containing 1-3 nitrogen atoms. In some embodiments, the FRAC group 3 fungicide is a piperazine, a pyridine, a pyrimidine, an imidazole, a triazole, a triazolinthione, or any combination thereof. In some embodiments, the FRAC group 3 fungicide is triforine, pyrifenox pyrisoxazole, fenarimol, nuarimol, imazalil, oxpoconazole, pefurazoate, prochloraz, triflumizole, azaconazole, bitertanol, bromuconazole, cyproconazole, difenoconazole, diniconazole, epoxiconazole, etaconazole, fenbuconazole, fluquinconazole, flusilazole, flutriafol, hexaconazole, imibenconazole, ipconazole, mefentrifluconazole, fluconazole, metconazole, myclobutanil, penconazole, propiconazole, simeconazole, tebuconazole, tetraconazole, triadimefon, triadimenol, triticonazole, prothioconazole, or any combination thereof. In some embodiments, the FRAC 3 group fungicide is a triazole, a metconazole, or a fluconazole. In some embodiments, the fungicide is a polyene macrolide fungicide. In some embodiments, the polyene macrolide fungicide targets ergosterol in a fungus, is an amphoteric macrolide, and/or is isolated from a Streptomyces species. In some embodiments, the polyene macrolide fungicide is natamycin (pimaricin), amphotericin, nystatin, or any combination thereof. In some embodiments, the polyene macrolide fungicide is natamycin. In some embodiments, the fungicide is an antifungal peptide that interacts with and disrupts the fungal cell membrane and/or the fungal cell wall. In some embodiments, the antifungal peptide is a cell membrane-interacting antifungal peptide and/or antimicrobial peptide (AMP). In some embodiments, the antifungal peptide is drosomycin, cecropin, diptericin, drosocin, metchnikowin, attacin, nisin, thanatin, a proline-rich peptide, a glycine-rich peptide, or any combination thereof. In some embodiments, the antifungal peptide is drosomycin. In some embodiments, the fungicide is a cyclic lipopeptide. In some embodiments, the cyclic lipopeptide inhibits beta-glucan synthesis in the fungal cell wall. In some embodiments, the cyclic lipopeptide is an echinocandin. In some embodiments, the cyclic lipopeptide is caspofungin, micafungin, anidulafungin, or any combination thereof. In some embodiments, the cyclic lipopeptide is caspofungin. In some embodiments, the fungicide is classified as a FRAC group 12 fungicide. In some embodiments, the fungicide is a phenylpyrrole fungicide with a mode of action that involves targeting signal transduction in a fungus. In some embodiments, the phenylpyrrole fungicide is fludioxonil or fenpiclonil, or a combination thereof. In some embodiments, the phenylpyrrole fungicide is fludioxonil or fenpiclonil. In some embodiments, the fungicide is classified as a FRAC group 7 fungicide or a FRAC group 11 fungicide. In some embodiments, the fungicide is a succinate dehydrogenase inhibitor (SDHI) that inhibits succinate dehydrogenase (SDH) and is classified as a FRAC group 7 fungicide. In some embodiments, the FRAC group 7 fungicide is an amide. In some embodiments, the FRAC group 7 fungicide is a benzamide, a pyridine amide, or a carboxamide. In some embodiments, the FRAC group 7 fungicide is a phenyl-benzamide, phenyl-oxo-ethyl thiophene amide, pyridinyl-ethyl-benzamide, phenyl-cyclobutyl-pyridineamide, furan-carboxamide, oxathiin-carboxamide, thiazole-carboxamide, pyrazole-4-carboxamide, N-cyclopropyl-N-benzyl-pyrazole-carboxamide, N-methoxy-(phenyl-ethyl)-pyrazole-carboxamide, pyridine-carboxamide, or pyrazine-carboxamide, or any combination thereof. In some embodiments, the FRAC group 7 fungicide is benodanil, flutolanil, mepronil, isofetamid, fluopyram, cyclobutrifluram, fenfuram, carboxin, oxycarboxin, thifluzamide, benzovindiflupyr, bixafen, fluindapyr, fluxapyroxad, furametpyr, inpyrfluxam, isopyrazam, penflufen, penthiopyrad, sedaxane, isoflucypram, pydiflumetofen, boscalid, or pyraziflumid, or any combination thereof. In some embodiments, the FRAC group 7 fungicide is a pyridine-carboxamide. In some embodiments, the FRAC group 7 fungicide is boscalid. In some embodiments, the fungicide is a quinone outside inhibitor (QoI) that inhibits respiration by targeting cytochrome bc1 (ubiquinol oxidase) at the Qo site and is classified as a FRAC group 11 fungicide. In some embodiments, the FRAC group 11 fungicide is a synthetic strobilurin analogue. In some embodiments, the FRAC group 11 fungicide is a FRAC group 11A fungicide. In some embodiments, the FRAC group 11 fungicide is a methoxy-acrylate, methoxy-acetamide, methoxy-carbamate, oximino-acetate, oximino-acetamide, oxazolidine-dione, dihydro-dioxazine, imidazolinone, benzyl-carbamate, or synthetic strobilurin analogue, or any combination thereof. In some embodiments, the FRAC group 11 fungicide is azoxystrobin, coumoxystrobin, enoxastrobin, flufenoxystrobin, picoxystrobin, pyraoxystrobin, mandestrobin, pyraclostrobin, pyrametostrobin, triclopyricarb, kresoxim-methyl, trifloxystrobin, dimoxystrobin, fenaminstrobin, metominostrobin, orysastrobin, famoxadone, fluoxastrobin, fenamidone, pyribencarb, or pyraziflumid, or any combination thereof. In some embodiments, the FRAC group 11 fungicide is an oximino-acetate. In some embodiments, the FRAC group 11 fungicide is trifloxystrobin. In some embodiments, the fungicide is classified as a FRAC group 9 fungicide. In some embodiments, the fungicide is an anilino-pyrimidine fungicide that targets methionine biosynthesis and is classified as a FRAC group 9 fungicide. In some embodiments, the FRAC 9 group fungicide is cyprodinil, mepanipyrim, pyrimethanil, or any combination thereof. In some embodiments, the FRAC 9 group fungicide is cyprodinil. In some embodiments, the antimicrobial peptide targets a different bacterial function or growth process than the antifungal agent.

In some embodiments of the foregoing, any combinations of one or more antimicrobial peptides with any of the fungicides described herein can be employed. For example, in certain embodiments, a combination of one or more fungicides from the azoles, polyene macrolides, AMPs, or cyclic lipopeptides can be used in combinations with one or more antimicrobial peptides.

In some embodiments, a composition comprises at least one antimicrobial peptide and a polyene antifungal (e.g., amphotericin B or nystatin) or a derivative thereof. In some embodiments, a composition comprises at least one antimicrobial peptide and amphotericin B or a derivative thereof. In some embodiments, combining at least one antimicrobial peptide with amphotericin B reduces the effective amount of amphotericin B, allowing a lower dose to be used. In some embodiments, a composition comprises at least one antimicrobial peptide and an azole antifungal (e.g., fluconazole or ketaconazole) or a derivative thereof. In some embodiments, a composition comprises at least one antimicrobial peptide and an echinocandin (e.g., caspofungin) antifungal or a derivative thereof.

In some embodiments, the composition comprises an antimicrobial peptide and antimycotic present in about an equal molar ratio, e.g., about 1:1. In some embodiments, the composition comprises an antimicrobial peptide and antimycotic present in an unequal molar ratio, e.g., about 1000:1, about 500:1, about 250:1, about 100:1, about 50:1, about 25:1, about 20:1, about 15:1, about 10:1, about 5:1, about 2:1, about 1:2, about 1:5, about 1:10, about 1:15, about 1:20, about 1:25, about 1:50, about 1:100, about 1:250, about 1:500, or about 1:1000.

One skilled in the art will appreciate that a suitable concentration of each antifungal agent in the composition depends on factors such as efficacy, stability of the antibiotic, number of distinct antibiotics, the formulation, and methods of application of the composition.

### 6. Cell Penetrating Peptides

In some aspects of the compositions described herein, the composition of one or more antimicrobial peptides further comprises at least one cell penetrating peptide (CPP). In some embodiments, the composition comprises a polynucleotide encoding at least one CPP. In some embodiments, the composition comprises at least one CPP selected from the group consisting of SEQ ID NOs: 385-398. In some embodiments, the one or more antimicrobial peptides and CPP are fused. The antimicrobial peptide and CPP may be fused by direct transcriptional fusion, translational fusion, or connected via covalent linkage (e.g., via a polypeptide linker).

In some embodiments, the composition comprises a linker associating the one or more antimicrobial peptides with at least one CPP. In some embodiments, the linker is a polypeptide linker.

### 7. Formulation

The formulation of the compositions described herein can be chosen from a number of formulation types, including isolated antimicrobial peptides, which may further be complexed with dustable powders (DP), soluble powders (SP), water soluble granules (SG), water dispersible granules (WG), wettable powders (WP), granules (GR) (slow or fast release), soluble concentrates (SL), oil miscible liquids (OL), ultra-low volume liquids (UL), emulsifiable concentrates (EC), dispersible concentrates (DC), emulsions (both oil in water (EW) and water in oil (EO)), microemulsions (ME), suspension concentrates (SC), oil-based suspension concentrate (OD), aerosols, fogging/smoke formulations, capsule suspensions (CS) and seed/plant treatment formulations.

In some embodiments the formulation comprises an effective amount of at least one antimicrobial peptide, antimicrobial peptide precursor, antimicrobial peptide fragment, or antimicrobial peptide motif, and that includes (a) an amino acid sequence of an antimicrobial peptide that has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity with a sequence selected from the group consisting of SEQ ID NO: 1-303 and 326-374 and 401-420; or (b) an amino acid sequence of an antimicrobial peptide motif; and a carrier. In some embodiments of this aspect, the antimicrobial peptide motif includes at least one of SEQ ID NO: 1-303 or 326-374 or 401-420. In additional embodiments of this aspect, which may be combined with any preceding embodiment, the antimicrobial peptide is active and/or toxic towards microbial cells. In some embodiments, the antimicrobial peptide is not toxic towards mammalian cells, e.g., human cells.

In further embodiments of this aspect, which may be combined with any preceding embodiment, the carrier is selected from an agriculturally acceptable carrier, or a pharmaceutically acceptable carrier. Yet another aspect of the disclosure relates to a composition comprising antimicrobial peptide, antimicrobial peptide precursor, the antimicrobial peptide fragment, or the antimicrobial peptide motif of any one of the preceding embodiments and an agriculturally acceptable carrier. In additional embodiments of this aspect, the agriculturally acceptable carrier includes one or more of an adjuvant, an inert component, a dispersant, a surfactant, a tackifier, a binder, or a stabilizer. Adjuvants and other components useful in agricultural formulations are described, e.g., in the Compendium of Herbicidal Adjuvants, 13th edition, 2016; available at siu-weeds[dot]com/adjuvants/index-adj[dot]html.

In some embodiments of this aspect, the composition is formulated as one of a seed treatment, a foliar spray treatment, a foliar drench treatment, a Ready-To-Use (RTU) formulation, a produce coating, a suspension concentrate, a tank-mix, an aerosol, a root dip, a drench, a fog, a soil treatment, an irrigation formulation, or a sprinkler formulation. In further embodiments of this aspect, the agriculturally acceptable carrier includes a solid carrier, a liquid carrier, a gel carrier, a suspension, or an emulsion.

In additional embodiments of this aspect, which may be combined with any preceding embodiment, the composition is formulated as a liquid, a gel, an emulsion, a suspension, an encapsulation, a solid, a powder, a coating, a spray, a soil drench, granules, a seed coat, or a bait. In some embodiments of this aspect, such agricultural formulations further include one or more additional components, such as an herbicide, insecticide, nematicide, fungicide (other than the antimicrobial peptide, antimicrobial peptide precursor, antimicrobial peptide fragment, or antimicrobial peptide motifs herein disclosed), attractant, or bait. In some embodiments of this aspect, the composition is formulated for application to human-built structures (e.g., buildings, fencing, walls) or artifacts (e.g., furniture, clothing, fabrics) or for incorporation in materials useful for making human-built structures or artifacts. In some embodiments of this aspect, the composition is incorporated as an addition to food or feed, e.g., products processed from plants. In additional embodiments of this aspect, the compositions are formulated as slow-release or controlled-release formulations.

In some embodiments, the carrier is a pharmaceutically acceptable carrier. Pharmaceutically acceptable carriers and excipients in the present compositions are nontoxic to recipients at the dosages and concentrations employed. Acceptable carriers and excipients can include buffers such as phosphate, citrate, HEPES, and TAE, antioxidants such as ascorbic acid and methionine, preservatives such as hexamethonium chloride, octadecyldimethylbenzyl ammonium chloride, resorcinol, and benzalkonium chloride, proteins such as human serum albumin, gelatin, and immunoglobulins, hydrophilic polymers such as dextran and polyvinylpyrrolidone, amino acids such as glycine, glutamine, histidine, and lysine, and carbohydrates such as glucose, mannose, sucrose, and sorbitol. The compositions can be formulated according to conventional pharmaceutical practice. In embodiments, the composition is formulated as a pharmaceutical (or veterinary) formulation such as, but not limited to, a liquid, emulsion, reverse emulsion, suspension, gel, cream, ointment, injectable, or solid, or any appropriate formulation, e.g., for topical, oral, intravenous, intramuscular, intraperitoneal, aerosolized, or nebulized administration, or for application to a subject by a device, such as a transdermal patch, bandage, tape, film, coating, or solid or porous matrix or surface. The concentration of the compound in the formulation will vary depending upon a number of factors, including the dosage of the active agent to be administered, and the route of administration.

Additional embodiments include formulations designed for agricultural, pharmaceutical, or veterinary use, wherein the antimicrobial peptide (or precursor thereof) is provided in a cell (viable and intact, or non-viable and intact) or as a cell-derived preparation, such as lyophilized cells, a suspension or pellet of lysed cells, or a cell membrane-bound particle or analogous synthetic lipid mono- or bilayer-bound particle (e.g., a minicell, exosome, liposome or vesicle; see, e.g., exosomes and vesicles described in Di Gioia et al. (2020) Open Medicine, 15:1096-1122, Mozafari (2010) “Nanoliposomes: Preparation and Analysis”, Chapter 2 in “Liposomes, Methods in Molecular Biology, volume 605”, Weissig (Ed.), DOI 10.1007/978-1-60327-360-2_2, and International Patent Application Publication WO2019/222379; also see, e.g., non-replicating minicells described in Nguyen (2011), “Cell Division Gene from Bacteria in Minicell Production for Therapy, Advances in Cancer Therapy, Gali-Muhtasib (Ed.), ISBN: 9789533077031, available at www[dot]intechopen[dot]com/books/advances-in-cancer-therapy/cell-division-gene-from-bacteria-in-minicell-production-for-therapy; Ni et al. (2021), ACS Synth. Biol., 10_1284-1291, and International Patent Application Publication WO2020/123569; all references cited in this paragraph are incorporated by reference in their entirety. Examples of such cell-derived formulations include fermentation preparations of bacterial, fungal, plant, or animal (e.g., insect) cells or minicells expressing one or more antimicrobial peptides and grown in culture; formulations made from such fermentation preparations can be provided (e.g., by spraying, soaking, painting, or injecting) to a subject organism or object or environment to provide protection from microbial infection or growth.

Still another aspect of the disclosure relates to a composition having antimicrobial properties, including a substrate or matrix that is complexed with at least one antimicrobial peptide, antimicrobial peptide precursor, antimicrobial peptide fragment of any preceding embodiments. The complexation between the substrate or matrix with at least one antimicrobial peptide, antimicrobial peptide precursor, antimicrobial peptide fragment, or antimicrobial peptide motif is through: (a) covalent bonding, (b) non-covalent bonding, or (c) a combination of (a) and (b). In some embodiments of this aspect, the substrate or matrix includes polypeptides. In additional embodiments of this aspect, which may be combined with any preceding embodiment that has a substrate or matrix, the substrate or matrix includes self-assembling peptides.

Any suitable substrate or matrix known to those in the art may be applied to the present disclosure. In some embodiments, the substrate or matrix comprises polypeptides. In some embodiments, the polypeptides are self-assembling peptides. Self-assembling peptide have been known to those of ordinary skill in the art, as demonstrated by Miki et al. (2021) Nature Communications, 21:3412, DOI: 10.1038/s41467-021-23794-6, which is specifically and entirely incorporated by reference herein for everything it teaches. In some embodiments, the complexation between the substrate or matrix and at least one antimicrobial peptide is through covalent bonding. In some embodiments, the complexation between the substrate or matrix and at least one antimicrobial peptide is through non-covalent bonding. In some embodiments, the complexation between the substrate or matrix and at least one antimicrobial peptide is through a combination of covalent bonding and non-covalent bonding.

### 8. Encapsulated Peptides

In some embodiments, the at least one antimicrobial peptide, antimicrobial peptide precursor, antimicrobial peptide fragment, or antimicrobial peptide motif at least one antimicrobial peptide, antimicrobial peptide precursor, antimicrobial peptide fragment, or antimicrobial peptide motif are incorporated in a liposome, e.g., a vesicle. In some embodiments, the liposome comprises a phospholipid monolayer. In some embodiments, the liposome comprises a phospholipid bilayer. In some embodiments, the liposome comprises a hydrophobic core. In some embodiments, the liposome comprises a hydrophilic core.

In some embodiments the liposome comprises an effective amount of at least one antimicrobial peptide, antimicrobial peptide precursor, antimicrobial peptide fragment, or antimicrobial peptide motif, and that includes (a) an amino acid sequence of an antimicrobial peptide that has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity with a sequence selected from the group consisting of SEQ ID NO: 1-303 and 326-374 and 401-420; or (b) an amino acid sequence of an antimicrobial peptide motif; and a carrier. In some embodiments of this aspect, the antimicrobial peptide motif includes at least one of SEQ ID NO: 1-303 or 326-374 or 401-420. In additional embodiments of this aspect, which may be combined with any preceding embodiment, the antimicrobial peptide is active and/or toxic towards microbial cells. In some embodiments, the antimicrobial peptide is not toxic towards mammalian cells, e.g., human cells.

In some embodiments, the liposome is a nature-derived lipid particle comprising at least one phospholipid, at least one non-polar lipid, and at least one surface modifier, wherein the lipid particle comprises a hydrophobic core. In some embodiments, the lipid particle comprising a hydrophilic core comprises a hydrophilic antimicrobial peptide of any of the preceding embodiments. In some embodiments, the liposome is a nature-derived lipid particle comprising at least one phospholipid, at least one non-polar lipid, and at least one surface modifier, wherein the lipid particle comprises a hydrophilic core. In some embodiments, the lipid particle comprising a hydrophilic core comprises a hydrophilic antimicrobial peptide of any of the preceding embodiments. In some embodiments, the lipid particle comprising a hydrophilic core comprises a polynucleotide encoding an antimicrobial peptide of any of the preceding embodiments.

Exemplary compositions of nature-derived lipid particles for encapsulation of the antimicrobial peptide of any of the preceding embodiments are described in WO2017153993A1, WO2021041301A1 and PCT/US2024/015064 which each are incorporated herein in their entireties.

### 9. Kits and Additives

The disclosure also provides packs or kits comprising one or more containers filled with one or more compounds to be administered in practicing the methods of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

Kits provided herein may include one or more containers, and instruction for use thereof according to the methods provided herein. Instructions supplied in the kits of the invention are typically written instructions on a label or package insert (e.g., a paper sheet included in the kit), but machine-readable instructions (e.g., instructions carried on a magnetic or optical storage disk) are also acceptable.

The kits provided herein are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

In a certain embodiment, the kit contains an effective amount of at least one antimicrobial peptide, antimicrobial peptide precursor, antimicrobial peptide fragment, or antimicrobial peptide motif, and that includes (a) an amino acid sequence of an antimicrobial peptide that has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity with a sequence selected from the group consisting of SEQ ID NO: 1-303 and 326-374 and 401-420; or (b) an amino acid sequence of an antimicrobial peptide motif; and a carrier. In some embodiments of this aspect, the antimicrobial peptide motif includes at least one of SEQ ID NO: 1-303 or 326-374 or 401-420. In additional embodiments of this aspect, which may be combined with any preceding embodiment, the antimicrobial peptide is active and/or toxic towards microbial cells. In some embodiments, the antimicrobial peptide is not toxic towards mammalian cells, e.g., human cells. In certain embodiments, the kit comprises a container conducive for use in conjunction with a tree injection system, e.g., the Trecise injection system as disclosed in WO2020021041, which is incorporated it its entirety herein.

In some embodiments, the disclosure provides an additive to a food product (e.g., bread, or a dietary supplement) or a personal care product (e.g., tooth paste), wherein the additive comprises an effective amount of an antimicrobial peptide of any of the preceding embodiments.

### II. METHODS

### 1. Methods of Making Antimicrobial Peptides

### a) Chemical Synthesis

In some embodiments the antimicrobial peptide, antimicrobial peptide precursor, or antimicrobial peptide fragment are chemically synthesized. Solid-phase peptide synthesis (SPPS) is a highly successful method introduced by Merrifield in 1963 (Merrifield, R. B. (1963) J. Amer. Chem. Soc. 85, 2149-2154), incorporated in its entirety herein.

Two exemplary methodologies for the assembly of peptide chains by SPSS can be used, e.g., the stepwise solid-phase synthesis, and solid-phase fragment condensation method. In stepwise SPPS, the C-terminal amino acid in the form of an N-a-protected, if necessary, sidechain, protected reactive derivative is covalently coupled either directly or by means of a suitable linker to a “solid”-support, e.g., a polymeric resin, which is swollen in an organic solvent. The N-a-protective group is removed, and the subsequent protected amino acids are added in a stepwise fashion. When the desired peptide chain length has been obtained, the side-chain protective groups are removed, and the peptide is cleaved from the resin. This may be done in separate steps or at the same time. In the solid-phase fragment condensation method, the target sequence is assembled by consecutive condensation of fragments on a solid support using protected fragments prepared by stepwise SPPS.

In some embodiments, coupling is performed using tert-butyloxycarbonyl (Boc) as the N-a-protective group. In some embodiments, 9-fluorenylmethyloxycarbonyl (Fmoc) introduced by Carpino and Han (Carpino, L. A. and Han, G. Y. (1972), J. Org. Chem. 37, 3404-3409) is used as the protective group.

The N-α-Boc-protected peptide coupled to a PAM-resin can be N-α-deprotected with trifluoroacetic acid (TFA). The resulting amine salt can be washed and neutralized with a tertiary amine. The subsequent peptide bond is formed by reaction with an activated Boc-amino acid, e.g., a symmetric anhydride. Generally, the side-chain protection is benzyl-based, and the deprotection is made with HF or a sulphonic acid. The N-α-Fmoc protected peptide coupled to a resin is N-α-deprotected by treatment with a secondary amine, normally piperidine, in an organic solvent, e.g., N,N-dimethyl formamide (DMF) or dichloromethane (DCM). After washing, the neutral peptide resin is reacted with an activated Fmoc-amino acid, e.g., a hydroxybenzotriazole active ester.

Methods used in the prior art to chemically synthesize peptides and proteins are reviewed in Kent, S. B. H. (1988), Ann. Rev. Biochem. 57, 957-989, incorporated in its entirety herein, and further in U.S. Pat. No. 7,348,404B2, incorporated in its entirety herein.

Peptides may be further purified from undesired peptide synthesis byproducts by using chromatographic protein separation methods, e.g., Reversed-Phase High Performance Liquid Chromatography (RP-HPLC) or ion-exchange chromatography, using the appropriate resins. Further, select ions can be exchanged for other more desirable counter ions by, e.g., ion-exchange chromatography.

### b) Expression

Methods of expressing a polypeptide, e.g., an antimicrobial peptide, are routine in the art. See, in general, Smales & James (Eds.), Therapeutic Proteins: Methods and Protocols (Methods in Molecular Biology), Humana Press (2005); and Crommelin, Sindelar & Meibohm (Eds.), Pharmaceutical Biotechnology: Fundamentals and Applications, Springer (2013). Methods for producing a polypeptide involve expression in plant cells, although recombinant proteins can also be produced using insect cells, yeast, bacteria, mammalian cells, or other cells under the control of appropriate promoters. Mammalian expression vectors may comprise nontranscribed elements such as an origin of replication, a suitable promoter and enhancer, and other 5′ or 3′ flanking nontranscribed sequences, and 5′ or 3′ nontranslated sequences such as necessary ribosome binding sites, a polyadenylation site, splice donor and acceptor sites, and termination sequences. DNA sequences derived from the SV40 viral genome, for example, SV40 origin, early promoter, enhancer, splice, and polyadenylation sites may be used to provide the other genetic elements required for expression of a heterologous DNA sequence. Appropriate cloning and expression vectors for use with bacterial, fungal, yeast, and mammalian cellular hosts are described in Green & Sambrook, Molecular Cloning: A Laboratory Manual (Fourth Edition), Cold Spring Harbor Laboratory Press (2012).

Various mammalian cell culture systems can be employed to express and manufacture a recombinant polypeptide agent. Examples of mammalian expression systems include CHO cells, COS cells, HeLA and BHK cell lines. Processes of host cell culture for production of protein therapeutics are described in, e.g., Zhou and Kantardjieff (Eds.), Mammalian Cell Cultures for Biologies Manufacturing (Advances in Biochemical Engineering/Biotechnology), Springer (2014). Purification of proteins is described in Franks, Protein Biotechnology: Isolation, Characterization, and Stabilization, Humana Press (2013); and in Cutler, Protein Purification Protocols (Methods in Molecular Biology), Humana Press (2010). Formulation of protein therapeutics is described in Meyer (Ed.), Therapeutic Protein Drug Products: Practical Approaches to formulation in the Laboratory, Manufacturing, and the Clinic, Woodhead Publishing Series (2012).

In some instances, a heterologous nucleic acid is encoding a polypeptide, e.g., an antimicrobial peptide. Nucleic acids encoding a polypeptide may have a length from about 10 to about 50,000 nucleotides (nts), about 25 to about 100 nts, about 50 to about 150 nts, about 100 to about 200 nts, about 150 to about 250 nts, about 200 to about 300 nts, about 250 to about 350 nts, about 300 to about 500 nts, about 10 to about 1000 nts, about 50 to about 1000 nts, about 100 to about 1000 nts, about 1000 to about 2000 nts, about 2000 to about 3000 nts, about 3000 to about 4000 nts, about 4000 to about 5000 nts, about 5000 to about 6000 nts, about 6000 to about 7000 nts, about 7000 to about 8000 nts, about 8000 to about 9000 nts, about 9000 to about 10,000 nts, about 10,000 to about 15,000 nts, about 10,000 to about 20,000 nts, about 10,000 to about 25,000 nts, about 10,000 to about 30,000 nts, about 10,000 to about 40,000 nts, about 10,000 to about 45,000 nts, about 10,000 to about 50,000 nts, or any range therebetween.

In some embodiments, the heterologous nucleic acid includes variants of a nucleic acid sequences expressing the peptide of interest. In some instances, the variant of the nucleic acids has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, e.g., over a specified region or over the entire sequence, to a sequence of a nucleic acid of interest. In some instances, the invention includes an active polypeptide encoded by a nucleic acid variant as described herein. In some instances, the active polypeptide encoded by the nucleic acid variant has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, e.g., over a specified region or over the entire amino acid sequence, to a sequence of a polypeptide of interest or the naturally derived polypeptide sequence.

Certain methods for expressing a nucleic acid encoding a protein may involve expression in cells, including insect, yeast, plant, bacteria, or other cells under the control of appropriate promoters. Expression vectors may include nontranscribed elements, such as an origin of replication, a suitable promoter and enhancer, and other 5′ or 3′ flanking nontranscribed sequences, and 5′ or 3′ nontranslated sequences such as necessary ribosome binding sites, a polyadenylation site, splice donor and acceptor sites, and termination sequences. DNA sequences derived from the SV40 viral genome, for example, SV40 origin, early promoter, enhancer, splice, and polyadenylation sites may be used to provide the other genetic elements required for expression of a heterologous DNA sequence. Appropriate cloning and expression vectors for use with bacterial, fungal, yeast, and mammalian cellular hosts are described in Green et al., Molecular Cloning: A Laboratory Manual, Fourth Edition, Cold Spring Harbor Laboratory Press, 2012.

In some embodiments, an alpha-factor variant peptides, are expressed in Pichia pastoris. The polynucleotide encoding an alpha-factor variant peptide selected from SEQ ID NOs 1-303 and 326-374 and 401-420 may be operably linked to a Pichia-compatible promoter, such as the alcohol oxidase 1 (AOX1) promoter, glyceraldehyde-3-phosphate dehydrogenase (GAP) promoter, or formaldehyde dehydrogenase (FLD1) promoter. A secretion signal, such as the Saccharomyces cerevisiae alpha-factor prepro-leader sequence, may be included to enable secretion of the alpha-factor variant peptide into the culture medium. In some embodiments, the alpha-factor variant peptide is secreted in mature form, while in others, a precursor is secreted and subsequently cleaved to release the mature peptide. The coding sequence may be codon-optimized for expression in P. pastoris, and expression may be induced with methanol or under constitutive conditions, depending on the promoter used. In certain embodiments, a linker or fusion sequence (e.g., 6×His tag) is also included to facilitate purification. Suitable expression vectors include, but are not limited to, pPICZ, pGAPZ, and derivatives thereof.

Genetic modification using recombinant methods is generally known in the art. A nucleic acid sequence coding for a desired gene can be obtained using recombinant methods known in the art, such as, for example by screening libraries from cells expressing the gene, by deriving the gene from a vector known to include the same, or by isolating directly from cells and tissues containing the same, using standard techniques. Alternatively, a gene of interest can be produced synthetically, rather than cloned.

Expression of natural or synthetic nucleic acids is typically achieved by operably linking a nucleic acid encoding the gene of interest to a promoter, and incorporating the construct into an expression vector. Expression vectors can be suitable for replication and expression in bacteria. Expression vectors can also be suitable for replication and integration in eukaryotes. Typical cloning vectors contain transcription and translation terminators, initiation sequences, and promoters useful for expression of the desired nucleic acid sequence.

Additional promoter elements, e.g., enhancers, regulate the frequency of transcriptional initiation. Typically, these are located in the region 30-110 base pairs (bp) upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1a (EF-1a). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter.

Alternatively, the promoter may be an inducible promoter. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

The expression vector to be introduced can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes may be used for identifying potentially transformed cells and for evaluating the functionality of regulatory sequences. In general, a reporter gene is a gene that is not present in or expressed by the recipient source and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., FEBS Letters 479:79-82, 2000). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In some instances, an organism may be genetically modified to alter expression of one or more proteins. Expression of the one or more proteins may be modified for a specific time, e.g., development or differentiation state of the organism. In one instance, the invention includes a composition to alter expression of one or more proteins, e.g., proteins that affect activity, structure, or function. Expression of the one or more proteins may be restricted to a specific location(s) or widespread throughout the organism.

### 2. Methods of Controlling or Preventing Growth of a Microbial Pathogen

A further aspect of the disclosure relates to methods of controlling a microbial pathogen, including delivering to the microbial pathogen or an environment thereof a bacterial composition including an effective amount of a composition comprising the antimicrobial peptide, the antimicrobial peptide precursor, the antimicrobial peptide fragment, or the antimicrobial peptide motif.

In some embodiments, the composition for controlling or preventing growth of a microbial pathogen comprises an effective amount of at least one antimicrobial peptide, antimicrobial peptide precursor, antimicrobial peptide fragment, or antimicrobial peptide motif, and that includes (a) an amino acid sequence of an antimicrobial peptide that has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity with a sequence selected from the group consisting of SEQ ID NO: 1-145; or (b) an amino acid sequence of an antimicrobial peptide motif; and a carrier. In some embodiments of this aspect, the antimicrobial peptide motif includes at least one of SEQ ID NO: 1-145. In additional embodiments of this aspect, which may be combined with any preceding embodiment, the antimicrobial peptide is active and/or toxic towards microbial cells. In some embodiments, the antimicrobial peptide is not toxic towards mammalian cells, e.g., human cells. In some embodiments, the composition further comprises at least one bactericidal, bacteriostatic, or antifungal agent.

In some embodiments, methods are provided for controlling or preventing growth of a microbial pathogen, the method comprising applying, to the microbial pathogen or a locus containing the microbial pathogen, a composition comprising an effective amount of at least one antimicrobial peptide, antimicrobial peptide precursor, antimicrobial peptide fragment, or antimicrobial peptide motif of any of the preceding embodiments. In some embodiments, the composition further comprises a bactericidal agent.

Yet another aspect of the disclosure relates to methods of preventing growth of a microbial pathogen on a surface, including treating the surface with a composition comprising an effective amount of at least one antimicrobial peptide, antimicrobial peptide precursor, antimicrobial peptide fragment, or antimicrobial peptide motif of any of the preceding embodiments. In some embodiments, the composition further comprises an antibacterial or antifungal agent. In further embodiments of this aspect, which may be combined with any preceding embodiments, the surface is a non-living surface or is the surface of a living organism.

Also provided herein is a method of preventing growth of a microbial pathogen on a surface or within a structure (e.g., a human-built structure or artifact). In some embodiments, the method comprises treating the surface or structure with a composition (e.g., a paint, coating, spray, or dip) comprising an effective amount of at least one antimicrobial peptide, antimicrobial peptide precursor, antimicrobial peptide fragment, or antimicrobial peptide motif of any of the preceding embodiments. In some embodiments, the composition further comprises an antibacterial or antifungal agent. In some embodiments, the surface is a surface of a living organism. In embodiments, the structure is a human-built structure or artifact, such as a building, fence, wall, furniture, fabric, or components thereof.

Further provided is a method of reducing the dose of an antibacterial or antifungal agent for treatment of an infection caused by a microbial pathogen in a subject, the method comprising administering to the subject a composition comprising the antibacterial or antifungal agent and an antimicrobial peptide of any of the preceding embodiments. In some embodiments, the antibacterial or antifungal agent and the antimicrobial peptide act cooperatively, that is when the effect of one agent is added on to the effect of the other agent. In some embodiments, the antibacterial or antifungal agent and the antimicrobial peptide act synergistically, that is when the effect of the combination of the antibacterial or antifungal agent and the antimicrobial peptide exceeds the effect of each of the agents individually combined. In some embodiments, the subject is a plant, e.g., a citrus plant.

### 3. Methods of Controlling or Preventing Bacterial Growth

A further aspect of the disclosure relates to methods of controlling a bacterial pathogen, including delivering to the bacterial pathogen or an environment thereof a bacterial composition including an effective amount of a composition comprising the antimicrobial peptide, the antimicrobial peptide precursor, the antimicrobial peptide fragment, or the antimicrobial peptide motif.

In some embodiments, the composition for controlling or preventing bacterial growth comprises an effective amount of at least one antimicrobial peptide, antimicrobial peptide precursor, antimicrobial peptide fragment, or antimicrobial peptide motif, and that includes (a) an amino acid sequence of an antimicrobial peptide that has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity with a sequence selected from the group consisting of SEQ ID NO: 1-303 and 326-374 and 401-420; or (b) an amino acid sequence of an antimicrobial peptide motif; and a carrier. In some embodiments of this aspect, the antimicrobial peptide motif includes at least one of SEQ ID NO: 1-303 or 326-374 or 401-420. In additional embodiments of this aspect, which may be combined with any preceding embodiment, the antimicrobial peptide is active and/or toxic towards microbial cells. In some embodiments, the antimicrobial peptide is not toxic towards mammalian cells, e.g., human cells. In some embodiments, the composition further comprises at least one bactericidal or bacteriostatic agent.

In some embodiments, at least one of the components in the composition of any of the preceding embodiments exerts bacteriostatic or bacteriocidal activity against bacteria. In some instances, the bacteria is an Acidovorax Avenae subsp., including e.g., Acidovorax Avenae subsp. Avenae (=Pseudomonas Avenae subsp. Avenae), Acidovorax Avenae subsp. cattleyae (=Pseudomonas cattleyae), or Acidovorax Avenae subsp. citrulli (=Pseudomonas pseudoalcaligenes subsp. citrulli, Pseudomonas Avenae subsp. citrulli)). In some instances, the bacterium is a Burkholderia spp., including e.g., Burkholderia andropogonis (=Pseudomonas andropogonis, Pseudomonas woodsii), Burkholderia caryophylli (=Pseudomonas caryophylli), Burkholderia cepacia (=Pseudomonas cepacia), Burkholderia gladioli (=Pseudomonas gladioli), Burkholderia gladioli pv. agaricicola (=Pseudomnas gladioli pv. agaricicola), Burkholderia gladioli pv. alliicola (e.g., Pseudomonas gladioli pv. alliicola), Burkholderia gladioli pv. gladioli (e.g., Pseudomonas gladioli, Pseudomonas gladioli pv. gladioli), Burkholderia glumae (e.g., Pseudomonas glumae), Burkholderia plantarii (e.g., Pseudomonas plantarii), Burkholderia solanacearum (e.g., Ralstonia solanacearum), or Ralstonia spp. In some instances, the bacterium is a Liberibacter spp., including Candidatus Liberibacter spec., including e.g., Candidatus Liberibacter asiaticus, Liberibacter africanus (Laf), Liberibacter americanus (Lam), Liberibacter asiaticus (Las), Liberibacter europaeus (Leu), Liberibacter psyllaurous, or Liberibacter solanacearum (Lso).

In some instances, the bacterium is a Corynebacterium spp, including e.g., Corynebacterium fascians, Corynebacterium flaccumfaciens pv. fiaccumfaciens, Corynebacterium michiganensis, Corynebacterium michiganense pv. tritici, Corynebacterium michiganense pv. nebraskense, or Corynebacterium sepedonicum. In some instances, the bacterium is a Erwinia spp, including e.g., Erwinia amylovora, Erwinia ananas, Erwinia carotovora (e.g., Pectobacterium carotovorum), Erwinia carotovora subsp. atroseptica, Erwinia carotovora subsp. carotovora, Erwinia chrysanthemi, Erwinia chrysanthemi pv. zeae, Erwinia dissolvens, Erwinia herbicola, Erwinia rhapontic, Erwinia stewartiii, Erwinia tracheiphila, or Erwinia uredovora. In some instances, the bacterium is a Pseudomonas syringae subsp., including e.g., Pseudomonas syringae pv. Actinidiae (Psa), Pseudomonas syringae pv. atrofaciens, Pseudomonas syringae pv. coronafaciens, Pseudomonas syringae pv. glycinea, Pseudomonas syringae pv. lachrymans, Pseudomonas syringae pv. maculicola, Pseudomonas syringae pv. papulans, Pseudomonas syringae pv. striafaciens, Pseudomonas syringae pv. syringae, Pseudomonas syringae pv. tomato, or Pseudomonas syringae pv. tabaci. In some instances, the bacterium is a Streptomyces spp., including e.g., Streptomyces acidiscabies, Streptomyces albidoflavus, Streptomyces candidus (e.g., Actinomyces candidus), Streptomyces caviscabies, Streptomyces collinus, Streptomyces europaeiscabiei, Streptomyces intermedius, Streptomyces ipomoeae, Streptomyces luridiscabiei, Streptomyces niveiscabiei, Streptomyces puniciscabiei, Streptomyces retuculiscabiei, Streptomyces scabiei, Streptomyces scabies, Streptomyces setonii, Streptomyces steliiscabiei, Streptomyces turgidiscabies, or Streptomyces wedmorensis. In some instances, the bacterium is a Xanthomonas axonopodis subsp., including e.g., Xanthomonas axonopodis pv. alfalfae (=Xanthomonas alfalfae), Xanthomonas axonopodis pv. aurantifolii (=Xanthomonas fuscans subsp. aurantifolii), Xanthomonas axonopodis pv. allii (=Xanthomonas campestris pv. allii), Xanthomonas axonopodis pv. axonopodis, Xanthomonas axonopodis pv. bauhiniae (=Xanthomonas campestris pv. bauhiniae), Xanthomonas axonopodis pv. begoniae (=Xanthomonas campestris pv. begoniae), Xanthomonas axonopodis pv. betlicola (=Xanthomonas campestris pv. betlicola), Xanthomonas axonopodis pv. biophyti (=Xanthomonas campestris pv. biophyti), Xanthomonas axonopodis pv. cajani (=Xanthomonas campestris pv. cajani), Xanthomonas axonopodis pv. cassavae (=Xanthomonas cassavae, Xanthomonas campestris pv. cassavae), Xanthomonas axonopodis pv. cassiae (=Xanthomonas campestris pv. cassiae), Xanthomonas axonopodis pv. citri (=Xanthomonas citri), Xanthomonas axonopodis pv. citrumelo (=Xanthomonas alfalfae subsp. citrumelonis), Xanthomonas axonopodis pv. clitoriae (=Xanthomonas campestris pv. clitoriae), Xanthomonas axonopodis pv. coracanae (=Xanthomonas campestris pv. coracanae), Xanthomonas axonopodis pv. cyamopsidis (=Xanthomonas campestris pv. cyamopsidis), Xanthomonas axonopodis pv. desmodii (=Xanthomonas campestris pv. desmodii), Xanthomonas axonopodis pv. desmodiigangetici (=Xanthomonas campestris pv. desmodiigangetici), Xanthomonas axonopodis pv. desmodiilaxiflori (=Xanthomonas campestris pv. desmodiilaxiflori), Xanthomonas axonopodis pv. desmodiirotundifolii (=Xanthomonas campestris pv. desmodiirotundifolii), Xanthomonas axonopodis pv. dieffenbachiae (=Xanthomonas campestris pv. dieffenbachiae), Xanthomonas axonopodis pv. erythrinae (=Xanthomonas campestris pv. erythrinae), Xanthomonas axonopodis pv. fascicularis (=Xanthomonas campestris pv. fasciculari), Xanthomonas axonopodis pv. glycines (=Xanthomonas campestris pv. glycines), Xanthomonas axonopodis pv. khayae (=Xanthomonas campestris pv. khayae), Xanthomonas axonopodis pv. lespedezae (=Xanthomonas campestris pv. lespedezae), Xanthomonas axonopodis pv. maculifoliigardeniae (=Xanthomonas campestris pv. maculifoliigardeniae), Xanthomonas axonopodis pv. malvacearum (=Xanthomonas citri subsp. malvacearum), Xanthomonas axonopodis pv. manihotis (=Xanthomonas campestris pv. manihotis), Xanthomonas axonopodis pv. martyniicola (=Xanthomonas campestris pv. martyniicola), Xanthomonas axonopodis pv. melhusii (=Xanthomonas campestris pv. melhusii), Xanthomonas axonopodis pv. nakataecorchori (=Xanthomonas campestris pv. nakataecorchori), Xanthomonas axonopodis pv. passiflorae (=Xanthomonas campestris pv. passiflorae), Xanthomonas axonopodis pv. patelii (=Xanthomonas campestris pv. patelii), Xanthomonas axonopodis pv. pedalii (=Xanthomonas campestris pv. pedalii), Xanthomonas axonopodis pv. phaseoli (=Xanthomonas campestris pv. phaseoli, Xanthomonas phaseoli), Xanthomonas axonopodis pv. phaseoli var. fuscans (=Xanthomonas fuscans), Xanthomonas axonopodis pv. phyllanthi (=Xanthomonas campestris pv. phyllanthi), Xanthomonas axonopodis pv. physalidicola (=Xanthomonas campestris pv. physalidicola), Xanthomonas axonopodis pv. poinsettiicola (=Xanthomonas campestris pv. poinsettiicola), Xanthomonas axonopodis pv. punicae (=Xanthomonas campestris pv. punicae), Xanthomonas axonopodis pv. rhynchosiae (=Xanthomonas campestris pv. rhynchosiae), Xanthomonas axonopodis pv. ricini (=Xanthomonas campestris pv. ricini), Xanthomonas axonopodis pv. sesbaniae (=Xanthomonas campestris pv. sesbaniae), Xanthomonas axonopodis pv. tamarindi (=Xanthomonas campestris pv. tamarindi), Xanthomonas axonopodis pv. vasculorum (=Xanthomonas campestris pv. vasculorum), Xanthomonas axonopodis pv. vesicatoria (=Xanthomonas campestris pv. vesicatoria, Xanthomonas vesicatoria), Xanthomonas axonopodis pv. Vignaradiatae (=Xanthomonas campestris pv. Vignaeradiatae), Xanthomonas axonopodis pv. vignicola (=Xanthomonas campestris pv. vignicola), or Xanthomonas axonopodis pv. vitians (=Xanthomonas campestris pv. vitians). In some instances, the bacterium is Xanthomonas campestris pv. musacearum, Xanthomonas campestris pv. pruni (=Xanthomonas arboricola pv. pruni), or Xanthomonas fragariae. In some instances, the bacteria is a Xanthomonas translucens supsp. (=Xanthomonas campestris pv. hordei) including e.g., Xanthomonas translucens pv. arrhenatheri (=Xanthomonas campestris pv. arrhenatheri), Xanthomonas translucens pv. cerealis (=Xanthomonas campestris pv. cerealis), Xanthomonas translucens pv. graminis (=Xanthomonas campestris pv. graminis), Xanthomonas translucens pv. phlei (=Xanthomonas campestris pv. phlei), Xanthomonas translucens pv. phleipratensis (=Xanthomonas campestris pv. phleipratensis), Xanthomonas translucens pv. poae (=Xanthomonas campestris pv. poae), Xanthomonas translucens pv. secalis (=Xanthomonas campestris pv. secalis), Xanthomonas translucens pv. translucens (=Xanthomonas campestris pv. translucens), or Xanthomonas translucens pv. undulosa (=Xanthomonas campestris pv. undulosa). In some instances, the bacterium is a Xanthomonas oryzae supsp., Xanthomonas oryzae pv. oryzae (=Xanthomonas campestris pv. oryzae), or Xanthomonas oryzae pv. oryzicola (=Xanthomonas campestris pv. oryzicola). In some instances, the bacterium is a Xylella fastidiosa from the family of Xanthomonadaceae.

In some embodiments, methods are provided for controlling a bacterial pathogen of any of the preceding embodiments, the method comprising applying, to the bacterial pathogen or a locus containing the bacterial pathogen, a composition comprising an effective amount of at least one antimicrobial peptide, antimicrobial peptide precursor, antimicrobial peptide fragment, or antimicrobial peptide motif of any of the preceding embodiments. In some embodiments, the composition further comprises a bactericidal agent.

An additional aspect of the disclosure relates to methods of controlling growth or reproduction of a bacteria, including providing the bacteria of any of the preceding embodiments with a composition comprising an effective amount of at least one antimicrobial peptide, antimicrobial peptide precursor, antimicrobial peptide fragment, or antimicrobial peptide motif of any of the preceding embodiments. In further embodiments of this aspect, which may be combined with any preceding embodiment, the composition is provided to the bacterium by directly contacting the fungus with the composition, or by delivering the composition to the environment of the bacteria.

Yet another aspect of the disclosure relates to methods of preventing growth of a bacteria of any of the preceding embodiments on a surface, including treating the surface with a composition comprising an effective amount of at least one antimicrobial peptide, antimicrobial peptide precursor, antimicrobial peptide fragment, or antimicrobial peptide motif of any of the preceding embodiments. In some embodiments, the composition further comprises an antibacterial agent. In further embodiments of this aspect, which may be combined with any preceding embodiments, the surface is a non-living surface or is the surface of a living organism.

Also provided herein is a method of preventing growth of bacteria of any of the preceding embodiments on a surface or within a structure (e.g., a human-built structure or artifact). In some embodiments, the method comprises treating the surface or structure with a composition (e.g., a paint, coating, spray, or dip) comprising an effective amount of at least one antimicrobial peptide, antimicrobial peptide precursor, antimicrobial peptide fragment, or antimicrobial peptide motif of any of the preceding embodiments. In some embodiments, the composition further comprises an antibacterial agent. In some embodiments, the surface is a surface of a living organism. In embodiments, the structure is a human-built structure or artifact, such as a building, fence, wall, furniture, fabric, or components thereof.

Further provided is a method of reducing the dose of an antibacterial agent (e.g., oxytetracycline) for treatment of an infection caused by a bacterium in a subject, the method comprising administering to the subject a composition comprising the antibacterial agent and an antimicrobial peptide of any of the preceding embodiments. In some embodiments, the antibacterial agent and the antimicrobial peptide act cooperatively, that is when the effect of one agent is added on to the effect of the other agent. In some embodiments, the antibacterial agent and the antimicrobial peptide act synergistically, that is when the effect of the combination of the antibacterial agent and the antimicrobial peptide exceeds the effect of each of the agents individually combined. In some embodiments, the subject is a plant, e.g., a citrus plant. In some embodiments, the dose of an antibacterial agent, e.g., oxytetracycline, to treat HLB is reduced by combining oxytetracycline with an antimicrobial peptide of any of the preceding embodiments.

Further provided herein is a method of reducing or preventing the development of resistance in a cell against an antibacterial agent, the method comprising administering to a cell a composition comprising an antibacterial agent and an antimicrobial peptide of any of the preceding embodiments. In some embodiments, the antibacterial agent and the antimicrobial peptide are provided in combination. In some embodiments the antibacterial agent and the antimicrobial peptide are provided alternately (e.g., in a first time span the antibacterial agent is provided, and in a next time span the antimicrobial peptide is provided, wherein the time span can be any time period between a day and a year).

### 4. Methods of Controlling or Preventing Fungal Growth

A further aspect of the disclosure relates to methods of controlling a fungal pathogen, including delivering to the fungal pathogen or an environment thereof a composition including an effective amount of the antimicrobial peptide, the antimicrobial peptide precursor, the antimicrobial peptide fragment, or the antimicrobial peptide motif.

In some embodiments, the methods for controlling or preventing fungal growth comprises applying, to the fungal pathogen or a locus containing the fungal pathogen, a composition comprising an effective amount of at least one antimicrobial peptide, antimicrobial peptide precursor, antimicrobial peptide fragment, or antimicrobial peptide motif, and that includes (a) an amino acid sequence of an antimicrobial peptide that has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity with a sequence selected from the group consisting of SEQ ID NO: 1-303 and 326-374 and 401-420; or (b) an amino acid sequence of an antimicrobial peptide motif; and a carrier. In some embodiments of this aspect, the antimicrobial peptide motif includes at least one of SEQ ID NO: 1-303 or 326-374 or 401-420. In additional embodiments of this aspect, which may be combined with any preceding embodiment, the antimicrobial peptide is active and/or toxic towards microbial cells. In some embodiments, the antimicrobial peptide is not toxic towards mammalian cells, e.g., human cells. In some embodiments, the composition further comprises an antifungal agent.

Fungi that cause fungal diseases in plants, including diseases caused by powdery mildew pathogens, are for example Blumeria species, for example Blumeria graminis; Podosphaera species, for example Podosphaera leucotricha; Sphaerotheca species, for example Sphaerotheca fuliginea; Uncinula species, for example Uncinula necator, diseases caused by rust disease pathogens, for example Gymnosporangium species, for example Gymnosporangium sabinae; Hemileia species, for example Hemileia vastatrix; Phakopsora species, for example Phakopsora pachyrhizi and Phakopsora meibomiae; Puccinia species, for example Puccinia recondite, P. triticina, P. graminis or P. striiformis or P. hordei; Uromyces species, for example Uromyces appendiculatus; diseases caused by pathogens from the group of the Oomycetes, for example Albugo species, for example Algubo candida; Bremia species, for example Bremia lactucae; Peronospora species, for example Peronospora pisi, P. parasitica or P. brassicae; Phytophthora species, for example Phytophthora infestans; Plasmopara species, for example Plasmopara viticola; Pseudoperonospora species, for example Pseudoperonospora humuli or Pseudoperonospora cubensis; Pythium species, for example Pythium ultimum; leaf blotch diseases and leaf wilt diseases caused, for example, by Alternaria species, for example Alternaria solani; Cercospora species, for example Cercospora beticola; Cladiosporium species, for example Cladiosporium cucumerinum; Cochliobolus species, for example Cochliobolus sativus (conidia form: Drechslera, Syn: Helminthosporium), Cochliobolus miyabeanus; Colletotrichum species, for example Colletotrichum lindemuthanium; Cycloconium species, for example Cycloconium oleaginum; Diaporthe species, for example Diaporthe citri; Elsinoe species, for example Elsinoe fawcettii; Gloeosporium species, for example Gloeosporium laeticolor, Glomerella species, for example Glomerella cingulata; Guignardia species, for example Guignardia bidwelli; Leptosphaeria species, for example Leptosphaeria maculans, Leptosphaeria nodorum; Magnaporthe species, for example Magnaporthe grisea; Microdochium species, for example Microdochium nivale; Mycosphaerella species, for example Mycosphaerella graminicola, M. arachidicola and M. fifiensis; Phaeosphaeria species, for example Phaeosphaeria nodorum; Pyrenophora species, for example Pyrenophora teres, Pyrenophora tritici repentis; Ramularia species, for example Ramularia collo-cygni, Ramularia areola; Rhynchosporium species, for example Rhynchosporium secalis; Septoria species, for example Septoria apii, Septoria lycopersii; Typhula species, for example Typhula incarnata; Venturia species, for example Venturia inaequalis; root and stem diseases caused, for example, by Corticium species, for example Corticium graminearum; Fusarium species, for example Fusarium oxysporum; Gaeumannomyces species, for example Gaeumannomyces graminis; Rhizoctonia species, such as, for example Rhizoctonia solani; Sarocladium diseases caused for example by Sarocladium oryzae; Sclerotium diseases caused for example by Sclerotium oryzae; Tapesia species, for example Tapesia acuformis; Thielaviopsis species, for example Thielaviopsis basicola; ear and panicle diseases (including corn cobs) caused, for example, by Alternaria species, for example Alternaria spp.; Aspergillus species, for example Aspergillus flavus; Cladosporium species, for example Cladosporium cladosporioides; Claviceps species, for example Claviceps purpurea; Fusarium species, for example Fusarium culmorum; Gibberella species, for example Gibberella zeae; Monographella species, for example Monographella nivalis; Septoria species, for example Septoria nodorum; diseases caused by smut fungi, for example Sphacelotheca species, for example Sphacelotheca reiliana; Tilletia species, for example Tilletia caries, T. controversa; Urocystis species, for example Urocystis occulta; Ustilago species, for example Ustilago nuda, U. nuda tritici; fruit rot caused, for example, by Aspergillus species, for example Aspergillus flavus; Botrytis species, for example Botrytis cinerea; Penicillium species, for example Penicillium expansum and P. purpurogenum; Sclerotinia species, for example Sclerotinia sclerotiorum; Verticilium species, for example Verticilium alboatrum; seed and soilborne decay, mould, wilt, rot and damping-off diseases caused, for example, by Alternaria species, caused for example by Alternaria brassicicola; Aphanomyces species, caused for example by Aphanomyces euteiches; Ascochyta species, caused for example by Ascochyta lends; Aspergillus species, caused for example by Aspergillus flavus; Cladosporium species, caused for example by Cladosporium herbarum; Cochliobolus species, caused for example by Cochliobolus sativus; (Conidiaform: Drechslera, Bipolaris Syn: Helminthosporium)·, Colletotrichu species, caused for example by Colletotrichum coccodes; Fusarium species, caused for example by Fusarium culmorum; Gibberella species, caused for example by Gibberella zeae; Macrophomina species, caused for example by Macrophomina phaseolina; Monographella species, caused for example by Monographella nivalis; Penicillium species, caused for example by Penicillium expansum; Phoma species, caused for example by Phoma lingam; Phomopsis species, caused for example by Phomopsis sojae; Phytophthora species, caused for example by Phytophthora cactorum; Pyrenophora species, caused for example by Pyrenophora graminea; Pyricularia species, caused for example by Pyricularia oryzae; Pythium species, caused for example by Pythium ultimum; Rhizoctonia species, caused for example by Rhizoctonia solani; Rhizopus species, caused for example by Rhizopus oryzae; Sclerotium species, caused for example by Sclerotium rolfsii; Septoria species, caused for example by Septoria nodorum; Typhula species, caused for example by Typhula incarnata; Verticillium species, caused for example by Verticillium dahliae; cancers, galls and witches' broom caused, for example, by Nectria species, for example Nectria galligena; wilt diseases caused, for example, by Monilinia species, for example Monilinia laxa; leaf blister or leaf curl diseases caused, for example, by Exobasidium species, for example Exobasidium vexans; Taphrina species, for example Taphrina deformans; decline diseases of wooden plants caused, for example, by Esca disease, caused for example by Phaemoniella clamydospora, Phaeoacremonium aleophilum and Fomitiporia mediterranea; Eutypa dyeback, caused for example by Eutypa lata; Ganoderma diseases caused for example by Ganoderma boninense; Rigidoporus diseases caused for example by Rigidoporus lignosus; diseases of flowers and seeds caused, for example, by Botrytis species, for example Botrytis cinerea; diseases of plant tubers caused, for example, by Rhizoctonia species, for example Rhizoctonia solani; Helminthosporium species, for example Helminthosporium solani; Club root caused, for example, by Plasmodiophora species, for example Plamodiophora brassicae; diseases caused by bacterial pathogens, for example Xanthomonas species, for example Xanthomonas campestris pv. oryzae; Pseudomonas species, for example Pseudomonas syringae pv. lachrymans; Erwinia species, for example Erwinia amylovora.

An additional aspect of the disclosure relates to methods of controlling growth or reproduction of a fungus, including providing the fungus with a composition of any of the preceding embodiments. In some embodiments, the composition further comprises an antifungal agent. In further embodiments of this aspect, which may be combined with any preceding embodiment, the composition is provided to the fungus by directly contacting the fungus with the composition, or by delivering the composition to the environment of the fungus.

Yet another aspect of the disclosure relates to methods of preventing growth of a fungus on a surface, including treating the surface with a composition of any of the preceding embodiments. In some embodiments, the composition further comprises an antifungal agent. In further embodiments of this aspect, which may be combined with any preceding embodiment, the surface is a non-living surface or is the surface of a living organism.

Also provided herein is a method of preventing growth of a fungus on a surface or within a structure (e.g., a human-built structure or artifact). In some embodiments, the method comprises treating the surface or structure with a composition (e.g., a paint, coating, spray, or dip) of any of the preceding embodiments. In some embodiments, the composition further comprises an antifungal agent. In some embodiments, the surface is a non-living surface. In some embodiments, the surface is a surface of a living organism. In embodiments, the structure is a human-built structure or artifact, such as a building, fence, wall, furniture, fabric, or components thereof.

Further provided is a method of reducing the dose of an antifungal agent (e.g., amphotericin B) for treatment of an infection caused by a fungus in a subject, the method comprising administering to the subject a composition comprising the antifungal agent and an antifungal peptide of any of the preceding embodiments. In some embodiments, the antifungal agent and the antifungal peptide act cooperatively, that is when the effect of one agent is added on to the effect of the other agent. In some embodiments, the antifungal agent and the antifungal peptide act synergistically, that is when the effect of the combination of the antifungal agent and the antifungal peptide exceeds the effect of each of the agents individually combined. In some embodiments, the subject is a human. In some embodiments, the dose of an antifungal agent, e.g., amphotericin B, to treat a fungal infection, e.g., candidiasis, is reduced by combining amphotericin B with an antimicrobial peptide of any of the preceding embodiments.

Further provided herein is a method of reducing or preventing the development of resistance in a cell against an antifungal agent, the method comprising administering to a cell a composition comprising an antifungal agent (e.g., an azole antimycotic agent) and an antimicrobial peptide of any of the preceding embodiments. In some embodiments, the antifungal agent and the antimicrobial peptide are provided in combination. In some embodiments the antifungal agent and the antimicrobial peptide are provided alternately (e.g., in a first time span the antifungal agent is provided, and in a next time span the antimicrobial peptide is provided, wherein the time span can be any time period between a day and a year).

### 5. Methods of Treating a Plant

### a) Plants and Diseases

In some embodiments, the one or more of the antimicrobial peptides described herein are capable of treating bacterial infections in plants, using any of the contacting or injection methods described below. In some embodiments, one or more of the antimicrobial peptides described herein act as a therapeutic agent for plants infected with and/or at risk of being infected by a bacterial pathogen, e.g., a gram-negative bacterial pathogen.

In some embodiments, the plants are treated with a composition comprising an effective amount of at least one antimicrobial peptide, antimicrobial peptide precursor, antimicrobial peptide fragment, or antimicrobial peptide motif, and that includes (a) an amino acid sequence of an antimicrobial peptide that has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity with a sequence selected from the group consisting of SEQ ID NO: 1-303 and 326-374 and 401-420; or (b) an amino acid sequence of an antimicrobial peptide motif; and a carrier. In some embodiments of this aspect, the antimicrobial peptide motif includes at least one of SEQ ID NO: 1-303 or 326-374 or 401-420. In additional embodiments of this aspect, which may be combined with any preceding embodiment, the antimicrobial peptide is active and/or toxic towards microbial cells. In some embodiments, the antimicrobial peptide is not toxic towards mammalian cells, e.g., human cells.

In some embodiments, one or more of the antimicrobial peptides identified herein are used as a therapeutic agent for plants infected with and/or at risk of being infected by Erwinia amylovora, Candidatus Liberibacter asiaticus (CLas), Xanthomonas citri and Xylella fastidiosa (Xj), the causative agents of Fire Blight, HLB, citrus canker and Pierce's disease, respectively. In some embodiments, the plants or plant parts are infected or are at risk of being infected with fungi, e.g., Botrytis, or Fusarium spp.

In some embodiments, one or more of the antimicrobial peptides identified herein are used as a therapeutic agent for plants infected with and/or at risk of being infected by Erwinia amylovora, the causative agent of Fire Blight. In some embodiments, one or more of the antimicrobial peptides identified herein are used as a therapeutic agent for plants infected with and/or at risk of being infected by Candidatus Liberibacter asiaticus (CLas), the causative agent of HLB. In some embodiments, one or more of the antimicrobial peptides identified herein are used a therapeutic agent for plants infected with and/or at risk of being infected by Xanthomonas citri, the causative agent of citrus canker. In some embodiments, one or more of the antimicrobial peptides identified herein are used a therapeutic agent for plants infected with and/or at risk of being infected by Xylella fastidiosa (Xj), the causative agent of Pierce's disease.

A variety of plants can be delivered or treated with an antimicrobial peptide composition described herein to combat a plant pest, e.g., a microbial plant pest. Plants that can be delivered an antimicrobial peptide composition (e.g., “treated”) in accordance with the present methods include whole plants and parts thereof, including, but not limited to, shoot vegetative organs/structures (e.g., leaves, stems and tubers), roots, flowers and floral organs/structures (e.g., bracts, sepals, petals, stamens, carpels, anthers and ovules), seed (including embryo, endosperm, cotyledons, and seed coat) and fruit (the mature ovary), plant tissue (e.g., meristematic tissue, vascular tissue, ground tissue, and the like) and cells (e.g., guard cells, egg cells, and the like), and progeny of same. Plant parts can further refer parts of the plant such as the shoot, root, stem, seeds, stipules, leaves, petals, flowers, ovules, bracts, branches, petioles, internodes, bark, pubescence, tillers, rhizomes, fronds, blades, pollen, stamen, and the like.

The class of plants that can be treated in a method disclosed herein includes the class of higher and lower plants, including angiosperms (monocotyledonous and dicotyledonous plants), gymnosperms, ferns, horsetails, psilophytes, lycophytes, bryophytes, and algae (e.g., multicellular or unicellular algae). Plants that can be treated in accordance with the present methods further include any vascular plant, for example monocotyledons or dicotyledons or gymnosperms, including, but not limited to alfalfa, apple (e.g., ‘Gala’ apple trees), Arabidopsis, banana, barley, canola, castor bean, chrysanthemum, clover, cocoa, coffee, cotton, cottonseed, corn, crambe, cranberry, crucifers, cucumber, Dendrobium, Dioscorea, eucalyptus, fescue, flax, Gladiolus, Liliaceae, linseed, millet, muskmelon, mustard, oat, oil palm, canola or oilseed rape, papaya, peanut, pineapple, ornamental plants, Phaseolus, potato, rapeseed, rice, rye, ryegrass, safflower, sesame, sorghum, soybean, sugarbeet, sugarcane, sunflower, strawberry, tobacco, tomato, turfgrass, wheat, and vegetable crops such as lettuce, celery, broccoli, cauliflower, cucurbits; fruit and nut trees, such as apple, pear, peach, orange, grapefruit, lemon, lime, pomelo, almond, pecan, walnut, hazel; vines, such as grapes (e.g., a vineyard), kiwi, hops; cannabis, fruit shrubs and brambles, such as raspberry, blackberry, gooseberry; forest trees, such as ash, pine, fir, maple, oak, chestnut, popular; with alfalfa, canola, castor bean, corn, cotton, crambe, flax, linseed, mustard, oil palm, oilseed rape, peanut, potato, rice, safflower, sesame, soybean, sugarbeet, sunflower, tobacco, tomato, and wheat. Plants that can be treated in accordance with the methods of the present invention include any crop plant, for example, forage crop, oilseed crop, grain crop, fruit crop, vegetable crop, fiber crop, spice crop, nut crop, turf crop, sugar crop, beverage crop, and forest crop. In certain instances, the crop plant that is treated in the method is a soybean plant. In other certain instances, the crop plant is wheat. In certain instances, the crop plant is corn. In certain instances, the crop plant is cotton. In certain instances, the crop plant is alfalfa. In certain instances, the crop plant is sugarbeet. In certain instances, the crop plant is rice. In certain instances, the crop plant is potato. In certain instances, the crop plant is tomato.

In certain instances, the plant is a crop. Examples of such crop plants include, but are not limited to, monocotyledonous and dicotyledonous plants including, but not limited to, fodder or forage legumes, ornamental plants, food crops, trees, or shrubs selected from Acer spp., Allium spp., Amaranthus spp., Ananas comosus, Apium graveolens, Arachis spp, Asparagus officinalis, Beta vulgaris, Brassica spp. (e.g., Brassica napus, Brassica rapa ssp. (canola, oilseed rape, turnip rape), Camellia sinensis, Canna indica, Cannabis sativa, Capsicum spp., Castanea spp., Cichorium endivia, Citrullus lanatus, Citrus spp., Cocos spp., Coffea spp., Coriandrum sativum, Corylus spp., Crataegus spp., Cucurbita spp., Cucumis spp., Daucus carota, Fagus spp., Ficus carica, Fragaria spp., Ginkgo biloba, Glycine spp. (e.g., Glycine max, Soja hispida or Soja max), Gossypium hirsutum, Helianthus spp. (e.g., Helianthus annuus), Hibiscus spp., Hordeum spp. (e.g., Hordeum vuigare), Ipomoea batatas, Juglans spp., Lactuca sativa, Linum usitatissimum, Litchi chinensis, Lotus spp., Luffa acutangula, Lupinus spp., Lycopersicon spp. (e.g., Lycopersicon esculenturn, Lycopersicon lycopersicum, Lycopersicon pyriforme), Malus spp., Medicago sativa, Mentha spp., Miscanthus sinensis, Morns nigra, Musa spp., Nicotiana spp., Olea spp., Oryza spp. (e.g., Oryza sativa, Oryza lati folia), Panicum miliaceum, Panicum virgatum, Passiflora edulis, Petroselinum crispum, Phaseolus spp., Pinus spp., Pistacia vera, Pisum spp., Poa spp., Populus spp., Prunus spp., Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis spp., Solanum spp. (e.g., Solanum tuberosum, Solarium integrifolium or Solarium lycopersicum), Sorghum bicolor, Sorghum halepense, Spinacia spp., Tamarindus indica, Theobroma cacao, Trifolium spp., Triticosecale rimpaui, Triticum spp. (e.g., Triticum aestivum, Triticum durum, Triticum turgidum, Triticum hybernum, Triticum macha, Triticum sativum or Triticum vuigare), Vaccinium spp., Vicia spp., Vigna spp., Viola odorata, Vitis spp., and Zea mays. In certain embodiments, the crop plant is rice, oilseed rape, canola, soybean, corn (maize), cotton, sugarcane, alfalfa, sorghum, or wheat.

In certain embodiments, the compositions and methods can be used to treat post-harvest plants or plant parts, food, or feed products. In some instances, the food or feed product is a non-plant food or feed product (e.g., a product edible for humans, veterinary animals, or livestock (e.g., mushrooms)). When applying the composition comprising at least one antimicrobial peptide to a harvested part of a plant (also referred to herein as post-harvest), application may be by a variety of treatment methods, e.g., dip, drip, drench, spray, or fog. In alternative embodiments, the harvested plant part has applied to it a composition, such as a film or membrane, containing the antimicrobial peptide, or is packaged in a container that includes the antimicrobial peptide. Such treatments, compositions, and containers are further useful for protecting foodstuffs (e.g., processed food products such as bakery goods or processed fruit or vegetables) from fungal growth and spoilage.

The plant or plant part for use in the present invention include plants of any stage of plant development. In certain instances, the delivery can occur during the stages of germination, seedling growth, vegetative growth, and reproductive growth. In certain instances, delivery to the plant occurs during vegetative and reproductive growth stages. Alternatively, the delivery can occur to a seed. The stages of vegetative and reproductive growth are also referred to herein as “adult” or “mature” plants.

Exemplary plant diseases which may be treated, with causative pathogen shown in parenthesis, include Alternaria Leaf and Fruit Spot (Alternaria alternata), Anthracnose (Colletotrichum acutatum), Leaf Blight (Seimatosporium lichenicola), Leaf Rust (Tranzschelia discolor), Scab (Cladosporium carpophilum), Shot Hole (Wilsonomyces carpophilus), Brown Rot Blossom Blight (Monilinia laxa, M. fructicola), Black Sigatoka (Mycosphaerella fijiensis), Yellow Sigatoka (Mycosphaerella musicola), Alternaria Fruit Rot (Alternaria spp.), Anthracnose Fruit Rot (Colletotrichum gloeosporoides), Botryosphaeria Canker (Botryosphaeria spp.), Leaf Spot and Blotch (Mycosphaerella spp., Septoria spp.), Mummyberry (Monilinia vaccinii-corymbosi), Phomopsis Leaf Spot, Twig Blight and Stem Canker (Phomopsis vaccinii), Powdery Mildew (Sphaerotheca spp.), Septoria Blight (Septoria spp.), Spur Blight (Didymella spp., Phoma spp.), Anthracnose (Spaceloma necator, Elsinoe veneta), Botryosphaeria Canker (Botryosphaeria dothidea), Colletotrichum Rot (Colletotrichum gloeosporioides), Leaf Spot and Blotch (Mycosphaerella spp., Septoria rubi, Sphaerulina rubi), Powdery Mildew (Sphaerotheca macularis, Microphaera spp., Oidium spp.), Rosette or Double Blossom of Blackberries (Cercosporella rubi), Spur Blight (Didymella applanata), Blackberry Rust (Phragmidium spp.), Anthracnose (Colletotrichum fragariae), Leather Rot (Phytophthora cactorum), Powdery Mildew (Sphaerotheca macularis), Botrytis grey mould on Foliage (Botrytis cinerea), Seedling Root Rot, Basal Stem Rot (Rhizoctonia solani), Cottonball (Monilinia oxycocci), Fruit Rots (Physalospora vaccinia, Glomerella cingulata, Coleophoma empetri), Lophodermium Twig Blight (Lophodermium spp.), Fairy Ring Suppression (Psilocybe spp.), Albinism (Alternaria alternata pv citri), Alternaria Leaf and Fruit Spot (Alternaria citri), Anthracnose (Colletotrichum acutatum, C. gloeosporioides), Cercospora Leaf Spot (Cercospora spp.), Diplodia Stem-End Rot (Diplodia natalensis), Greasy Spot (Mycosphaerella citri), Melanose (Diaporthe citri), Penicillium Decays, Green Mold, Whisker Mold, Blue Mold (Penicillium spp.), Phomopsis Stem-End Rot (Phomopsis citrii), Post Bloom Fruit Drop (PFD) (Colletotrichum acutatum), Powdery Mildew (Erysiphe spp.), Scab (Elsinoe fawcettii), Sweet Orange Scab (Elsinoe australis), Black Spot (Guignardia citricarpa), Black Rot (Guignardia bidwellii), Downy Mildew (Plasmopara viticola), Phomopsis Cane and Leaf Spot (Phomopsis viticola), Powdery Mildew (Uncinula necator), Botrytis Bunch Rot (Botrytis cinerea), Aspergillus Crown Rot (Aspergillus niger), Pythium Damping Off (Pythium spp.), Stem Rot/White Mold (Sclerotium rolfsii), Rhizoctonia Peg and Pod Rot (Rhizoctonia solani), Stem Rot/White Mold (Sclerotium rolfsii), Cylindrocladium Black Rot (Cylindocladium crotalariae), Pythium Pod Rot (Pythium myriotylum), Alternaria Late Blight (Alternaria alternata), Botryosphaeria Panicle and Shoot Blight (Botryosphaeria dothidea), Septoria Leaf Spot (Septoria pistaciarum), Scab (Cladosporium carpophilum), Alternaria Spot and Fruit Rot (Alternaria alternata), Anthracnose (Colletotrichum prunicola, C. gloeosporioides), Leaf Rust (Tranzschelia discolor), Powdery Mildew (Sphaerotheca pannosa, Podosphaera clandestina), Shot Hole (Wilsonomyces carpophilus), Alternaria Leaf Spot (Alternaria spp., A. alternata), Ascochyta Leaf Spot (Ascochyta cynarae), Phyllostica Leaf Spot (Phyllostica spp.), Rust (Uromyces betae, Puccinia helianthi), White Rust (Albugo tragopogonis), Anthracnose (Colletotrichum acutatum, Glomerella cingulata), Eastern Filbert Blight (Anisogramma anomale), Late Blight (Alternaria alternata), Scab (Cladosporium carpophilum), Septoria Leaf Spot (Septoria pistaciarum), Shot Hole (Wilsonomyces carpophilus), Blossom Blight (Monilinia laxa, M. fructicola), Powdery Mildew (Erysiphe spp.), Rust (Puccinia spp.), Alternaria black spot (Alternaria brassicae), Black leg/Phoma (Leptosphaeria maculans), Cercospora leaf spot (C. beticola), Head rot (Rhizoctonia solani), Leaf spot and pod rot (Alternaria alternata), Powdery mildew (Erysiphe polygoni), Southern blight (Sclerotium rolfsii), Anthracnose leaf blight (Colletotrichum graminicola), Gray leaf spot (Cercospora sorghi), Northern corn leaf blight (Setosphaeria turcica), Northern corn leaf spot (Cochliobolus carbonum), Common Rust (Puccinia sorghi), Southern Rust (P. polysora), Southern corn leaf blight (Cochliobolus heterostrophus), Eye spot (Aureobasidium zeae), Physoderma brown spot (P. maydis), Yellow Leaf Blight (Phyllosticta maydis), Ascochyta blight (A. gossypii), Rust (Puccinia schedonnardi, P. cacabata), Rhizoctonia leaf and stem diseases (R. solani), Target spot (Corynespora cassiicola), Southern blight (Sclerotium rolfsii), Rhizoctonia limb rot (R. solani), Cylindrocladium black rot (C. crotalaria), White mold (Sclerotinia minor), Early leaf spot (Cercospora arachidicola), Late leaf spot (Cercosporidium personatum), Web blotch (Phoma arachidicola), Rust (Puccinia arachidis), Pepper Spot (Leptospherulina crassiasca), Southern stem rot (Sclerotium rolfsii), Rhizoctonia limb rot (R. solani), Cylindrocladium black rot (C. crotalaria), White mold (Sclerotinia minor), Anthracnose (Colletotrichum lindemuthianum), Ascochyta blight (A. phaseolorum), Cercospora leaf blotch (C. cruenta), Downy mildew (Phytophthora nicotianae), Rust (Uromyces appendiculatus), Anthracnose (ripe rot) (C. gloeosporoides), Mummy berry (M. vacciniicorymbosi), Rust (Pucciniastrum vaccinii), Septoria leaf spot (Septoria albopunctata), Downy mildew (Peronospora parasitica), Alternaria leaf blight (A. dauci), Cercospora leaf spot (C. carotae), Basal stalk rot (Rhizoctonia solani), Early blight (Cercospora apii), Late blight (Septoria apicola), Verticihium brown spot and dry bubble, Pink rot (Sclerotinia sclerotiorum), Lophodermium leaf/twig blight (hypophyllum), Upright dieback (Phomopsis vaccinii), Anthracnose (Colletotrichum spp.), Downy mildew (Pseudoperonospora cubensis), Target spot (Corynespora cassiicola), Alternaria leaf blight (A. cucumerina), Alternaria leaf spot (A. altemata), Cercospora leaf spot (C. citrullina), Gummy stem blight/vine decline (Didymella bryoniae), Powdery mildew (Sphaerotheca only), Scab (Cladosporium cucumerinum), Anthracnose (Colletotrichum spp.), Botrytis leaf mold (Botrytis cinerea), Cercospora leaf spot (Cercospora spp.), Powdery mildew (Leveillula taurica), Purple blotch (Alternaria porri), Botrytis neck rot, Downy mildew (Peronospora destructor), Early leaf spot (Cercospora arachidicola), Late leaf spot (Cercosporidium personatum), Pepper spot (Leptosphaerulina crassiasca), Black dot (Colletotrichum coccodes), Botrytis vine rot (B. cinerea), Early blight (Alternaria solani), Late blight (Phytophthora infestans), Anthracnose (Colletotrichum truncatum), Cercospora leaf blight (C. kikuchii), Diaporthe pod and stem rot (D. phaseolorum), Frogeye leaf spot (Cercospora sojina), Purple seed stain (C. kikuchii), Septoria brown spot (S. glycines), Rust (Phakopsora pachyrhizi), Stem canker (Diaporthe phaseolorum), Early blight (Alternaria solani), Gray leaf mold (Fluvia fluva Cladosporium), Gray leaf spot (Stemphyllium botryosum), Late blight (Phytophthora infestans), Septoria leaf spot (S. lycopersici), Target spot (Corynespora cassiicola), Alternaria fruit rot (black mold) (A. altemata), Anthracnose (Colletotrichum spp.), Botrytis gray mold (B. cinerea), Late blight fruit rot (P, infestans), Rhizoctonia fruit rot (R. solani), Anthracnose (Colletotrichum gloeosporioides), Anthracnose (Colletotrichum acutatum), Blossom blight/brown rot (Monilinia spp.), Scab (Venturia carpophila), Shot hole (Wilsonomyces carpophilus), Leaf curl (Taphrina deformans), Black knot (cherry, plum) (Apiosporina morbosa), Cherry leaf spot (Blumeriella jaapii), Scab (Cladosporium carpophilum), Interior needle blight (Mycosphaerella spp. and Phaeocryptopus nudus), Swiss needlecast (Phaeocryptopus gaeumannii), Interior needle blight (Mycosphaerella spp. and Phaeocryptopus nudus), Scleroderris canker (Gremmeniella abietina), Leaf rust (Thekopsora minima), Powdery mildew (Erysiphe necator), Alternaria rot (A. altemata), Angular leaf spot (Mycosphearella angulata), Anthracnose (Elsinoe ampelina), Black Rot (Guignardia bidwellii), Leaf Blight (Pseudocercospora vitis), Phomopsis cane and leaf spot (P. viticola), Rotbrenner (Pseudopezicula tracheiphila), Septoria leaf spot (S. ampelina), Apple Scab (Venturia inaequalis), Pear Scab (V. piris), Alternaria blotch, Alternaria rot (Alternaria spp.), Cedar apple rust (Gymnosporangium juniper-virginianae), Powdery mildew (Podosphaera leucotricha), Quince rust (Gymnosporangium spp.), Flyspeck and Sooty blotch, Bitter rot (Glomerella cingulata), Black rot (Botryosphaeria obtusa), Brooks fruit spot (Mycosphaerella pomi), White rot (Botryosphaeria dothidea), Alternaria rot and surface mold, Bitter rot, Blue mold, Bull's-eye rot, Gray mold, Phacidiopycnis rot, Rhizopus rot, Speck rot, Sphaeropsis rot, White rot, Damping off (Pythium spp.), Root Rot (Phytophthora spp.), Leather rot (P. cactorum), Red stele (P. fragariae), Vascular collapse (P. cactorum), Basal stem rot (Phytophthora spp.), Crown rot (Phytophthora capsici), Downy Mildew (Peronospora effuse, P. farinosa), White rust (Albugo occidentalis), Pink rot (Phytophthora erythroseptica), Pythium leak, Pythium seedling disease (Pythium spp.), Phytophthora root and stem rot (Phytophthora megasperma), Pythium damping off (Pythium spp.), Collar rot, Crown rot, Root rot (Phytophthora spp.), Crown rot, Spear rot (Phytophthora spp.), Root Rot (Phytophthora cinnamomi), Downy mildew (Peronospora parasitica), Brown rot, Citrus foot rot, Gummosis, Root rot, Trunk canker (Phytophthora spp.), or Downy mildew (Bremia lactucae). From an agricultural or horticultural perspective, and for the purposes of this application, some of the pathogens and diseases listed above are considered “fungal” although the causative pathogen is technically an oomycete (phylum Oomycota), including, but not limited to Pythion spp., Phytophthora spp., Peronospora spp., Plasmopara spp., Albugo spp., and Bremia spp.

Fungal diseases on leaves, stems, pods and seeds caused, for example, by Alternaria leaf spot (Alternaria tenuissima), Anthracnose (Colletotrichum gloeosporoides dematium var. truncatum), brown spot (Septoria glycines), Cercospora leaf spot and blight (Cercospora kikuchii), choanephora leaf blight (Choanephora infundibulifera trispora (Syn.)), dactuliophora leaf spot (Dactuliophora glycines), downy mildew (Peronospora manshurica), drechslera blight (Drechslera glycines), frogeye leaf spot (Cercospora sojina), leptosphaerulina leaf spot (Leptosphaerulina trifolii), Phyllostica leaf spot (Phyllosticta sojaecola), pod and stem blight (Phomopsis sojae), powdery mildew (Microsphaera diffusa), pyrenochaeta leaf spot (Pyrenochaeta glycines), Rhizoctonia aerial, foliage, and web blight (Rhizoctonia solani), rust (Phakopsora pachyrhizi, Phakopsora meibomiae), scab (Sphaceloma glycines), stemphylium leaf blight (Stemphylium botryosum), target spot (Corynespora cassiicola).

Fungal diseases on roots and the stem base caused, for example, by black root rot (Calonectria crotalariae), charcoal rot (Macrophomina phaseolina), Fusarium blight or wilt, root rot, and pod and collar rot (Fusarium oxysporum, Fusarium orthoceras, Fusarium semitectum, Fusarium equiseti), mycoleptodiscus root rot (Mycoleptodiscus terrestris), neocosmospora (Neocosmospora vasinfecta), pod and stem blight (Diaporthe phaseolorum), stem canker (Diaporthe phaseolorum var. caulivora), Phytophthora rot (Phytophthora megasperma), brown stem rot (Phialophora gregata), Pythium rot (Pythium aphanidermatum, Pythium irregulare, Pythium debaryanum, Pythium myriotylum, Pythium ultimum), Rhizoctonia root rot, stem decay, and damping-off (Rhizoctonia solani), sclerotinia stem decay (Sclerotinia sclerotiorum), sclerotinia southern blight (Sclerotinia rolfsii), thielaviopsis root rot (Thielaviopsis basicola).

In certain instances, the fungus is a Sclerotinia spp (Sclerotinia sclerotiorum). In certain instances, the fungus is a Botrytis spp (e.g., Botrytis cinerea). In certain instances, the fungus is an Aspergillus spp. In certain instances, the fungus is a Fusarium spp. In certain instances, the fungus is a Penicillium spp.

Compositions of the present invention are useful in various fungal control applications. The above-described compositions may be used to control fungal phytopathogens prior to harvest or postharvest fungal pathogens. In one embodiment, any of the above-described compositions are used to control target pathogens such as Fusarium species, Botrytis species, Verticillium species, Rhizoctonia species, Trichoderma species, or Pythium species by applying the composition to plants, the area surrounding plants, or edible cultivated mushrooms, mushroom spawn, or mushroom compost. In another embodiment, compositions of the present invention are used to control post-harvest pathogens such as Penicillium, Geotrichum, Aspergillus niger, or Colletotrichum species.

In some embodiments, the compositions as disclosed herein are useful in treating citrus greening, or Huanglongbing (HLB). HLB is a devastating plant disease, caused by Candidates Liberibacter asiaticus (CLas or Liberibacter), a gram-negative bacterium, which is transmitted by Asian citrus psyllids (ACP). In one specific embodiment, disclosed herein are novel systems, methods, and compositions for the treatment of HLB disease caused by CLas in plants. In some embodiments, the compositions comprising antimicrobial peptides as described herein are used to treat susceptible or already infected citrus plants, and cure, or lower the bacterial load and increase the productive years of the plant infected with, or at risk of being affected with HLB.

### b) Means of Contacting a Plant

In some embodiments, the disclosure provides methods for contacting the plant with the antimicrobial peptide compositions of any of the preceding embodiments, e.g., topically contacting the plant with the antimicrobial peptide compositions discussed herein. Administration generally is achieved by application of the compositions in a vehicle compatible with the plant to be treated (e.g., a botanically compatible vehicle or carrier), such as an aqueous vehicle, to the plant or to the soil surrounding the plant or by injection into the plant. Any application can be used; however, one application method includes trunk injection and foliar spraying as described herein. Other methods include application to the soil surrounding the plant, by injection, soaking or spraying, so that the applied compounds can come into contact with the plant roots and can be taken up by the roots. Additional topical applications may also be contemplated. The compositions disclosed herein can be formulated for seed or plant treatments in any of the following modes: dry powder, water slurriable powder, liquid solution, flowable concentrate or emulsion, emulsion, microcapsules, gel, or water dispersible granules.

In another embodiment, delivery of the antimicrobial peptide composition to plants can be via different routes. The compositions can be suitably administered as an aerosol, for example by spraying onto leaves or other plant material. The particles can also be administered by injection, for example directly into a plant, such as into the stem. In certain embodiments the compositions are administered to the roots. This can be achieved by spraying or watering plant roots with compositions. In other embodiments, the particles are introduced into the xylem or phloem, for example by injection or being included in a water supply feeding the xylem or phloem. Application to the stems or leaves of the plant can be performed by spraying or other direct application to the desired area of the plant; however, any method known in the art can be used. A solution or vehicle containing the antimicrobial peptides at a dosage of active ingredient can be applied with a sprayer to the stems or leaves until runoff to ensure complete coverage, and repeat three or four times in a growing season. The concentrations, volumes and repeat treatments may change depending on the plant.

In certain embodiments, the method comprises delivering a formulation comprising at least one antimicrobial peptide, and optionally one or more nutrients, into, e.g., a citrus plant. In certain embodiments the method comprises precision delivery (also referred to as “precision injection”) of a formulation into the citrus plant. Precision delivery refers to delivering the formulation only or substantially only into a target location in the citrus plant. For example, in some embodiments, the target location is the active vasculature of the plant. In certain embodiments, the method comprises injecting an injection formulation into and no further than the active vasculature of the plant. In some embodiments, the composition enters the active vasculature and is transported throughout the plant. In some variations, the active vasculature of the plant is the xylem and/or the phloem. In one variation, the active vasculature is active xylem (such as sapstream) and phloem. In further embodiments, precision delivery involves delivering the formulation into the active vasculature of the citrus plant while minimizing damage to the plant relative to traditional forms of injection drilling systems. In yet other embodiments, precision delivery involves using a system that can be configured to deliver formulation into and no further than the active vasculature of a plant.

The methods described herein comprise contacting the plant with the antimicrobial peptide compositions of any of the preceding embodiments at one or more stages of growth, including contacting a seed with an antimicrobial peptide composition. In some embodiments, the method comprises contacting the plant with the antimicrobial agents described herein in any order, e.g., contacting the plant first with an antimicrobial peptide, then contacting the plant with an antibacterial or antifungal agent or contacting the plant first with an antibacterial or antifungal agent, then contacting the plant with an antimicrobial peptide. In some embodiments, the antimicrobial peptide and antibacterial or antifungal agents are applied simultaneously.

In some aspects, the methods described herein comprise detecting the biodistribution of the antimicrobial peptide compositions administered to plants (e.g., detecting distribution throughout the trunk, stem, and other parts of a plant). Various means may be used for detection of biodistribution in a plant. In some embodiments, the antimicrobial peptides described herein further comprise a fluorescent label for use in detecting biodistribution of the antimicrobial peptide compositions. The fluorescent label may be incorporated at multiple positions of the peptide, including at the N-terminus of the peptide, the C-terminus of the peptide, or internally to the peptide. Multiple fluorescent labels may be used for one peptide or in one composition. Exemplary fluorescent labels and methods of labeling proteins are disclosed in Toseland (2013), Fluorescent labeling and modification of proteins, J Chem Biol. 6 (3): 85-95, Boaro et al. (2020), Light-Emitting Probes for Labeling Peptides, Cell Rep Phys Sci 1 (12): 100257, and Siepi et al. (2021), Environment-Sensitive Fluorescent Labelling of Peptides by Luciferin Analogues, Int J Mol Sci 22 (24): 13312. In some embodiments, the antimicrobial peptide compositions described herein further comprise a dye for use in detecting biodistribution of the antimicrobial peptide composition.

### i) Injection Technology

In some embodiments, the composition of any of the preceding embodiments is applied to the plant or plant part by injection into the plant or plant part. The injecting of the composition is performed, e.g., using an injection system comprising an injection tool operatively connected to a fluid delivery unit, wherein the fluid delivery unit is configured to deliver the injection formulation. In some embodiments, the injecting of the injection formulation comprises piercing the trunk or stem of a plant, e.g., a citrus plant using the injection tool of the injection system. In some embodiments, the injecting of the injection formulation comprises delivering at least a portion of the injection formulation from the fluid delivery unit through the injection tool into and no further than the active vasculature of the plant. Exemplary injection technologies are disclosed in WO2020021041, which is incorporated in its entirety herein.

In some embodiments, this disclosure also provides systems and devices for delivering injection formulations to the interior of the plant. In some embodiments, the systems comprise an injection tool operatively connected to a fluid delivery unit, wherein the injection tool is configured for precision delivery of the injection formulation to a target location inside the plant. In some embodiments, the systems are configured for precision delivery of an injection formulation into the active vasculature of a plant, e.g., a citrus plant. In some embodiments, the fluid delivery unit further comprises the formulation. In other embodiments, the system comprises an injection tool, a fluid delivery unit, and a source of source of formulation in fluid communication with the fluid delivery unit.

In some embodiments, the injection systems comprise an injection tool, a fluid delivery unit, and an injection formulation source. In operation, the injection tool is operatively connected to the fluid delivery unit such that injection formulation flows from the source through the injection tool into the plant. In some embodiments, the source of injection formulation is independent of the fluid delivery unit. In other embodiments, the source of injection formulation is integral with the fluid delivery unit. Certain embodiments of the injection systems are suitable for use in the methods described herein are described in further detail below.

In some embodiments, an injection system is used to deliver the injection formulation to a plant, e.g., a citrus plant. In some variations, the injection system comprises: an injection tool operatively connected to a fluid delivery unit. In certain variations, the injection tool comprises: a base having at least one inlet; and a body comprising at least one distribution reservoir, and at least one outlet. In some embodiments, the injection system comprises: an injection tool, a fluid delivery unit, and a source of active ingredient (including, for example, nutrients) formulated as a liquid.

In some variations, the body is shaped to pierce the plant, such as the trunk or stem of the plant. In certain variations, the body is in the shape of a blade. In certain variations, the body has a cutting edge at the tip of the body, and the width of the cutting edge is narrower than width of the body in the area connected to the base.

In certain variations, the body comprises: at least one outlet that receives the injection formulation from the at least one inlet, and at least one distribution reservoir that retains the injection formulation proximate to adjacent tissue of the plant. In certain variations, the fluid delivery unit is configured to store and deliver the injection formulation. In certain variations, the fluid delivery unit comprises a pressurized container (e.g., a pressurized canister).

In some embodiments, the method comprises: piercing the trunk or stem of a plant, e.g., a citrus plant, using the injection tool of the injection system; and delivering at least a portion of the injection formulation from the fluid delivery unit through the injection tool to the vasculature of the plant. In some variations, the injection formulation is delivered pneumatically or hydraulically.

In some embodiments, the injection formulation is precisely delivered. In some variations, the injection formulation is delivered into and no further than the active vasculature of the plant when the injection tool is inserted into the trunk or stem of the plant. In one variation, the injection formulation is delivered into and no further than the xylem, or the phloem or both of the plant when the injection tool is inserted into the trunk or stem of the plant.

In other embodiments, precisely delivering the injection formulation comprises inserting the injection tool into and no further than the active vasculature of the plant. In certain variations, precisely delivering the injection formulation comprises inserting the body of the injection tool into and no further than the active vasculature of the plant. In one variation, precisely delivering the injection formulation comprises inserting the injection tool such that the distribution reservoir is positioned in and no further than the active vasculature of the plant.

In some variations, the methods deliver at least 50%, at least 60%, at least 70%, at least 80%, at least 90%, at least 95%, at least 96%, at least 97%, at least 98% or at least 99% of the injection formulation into to the active vasculature of the plant. In one variation, the methods deliver at least 50%, at least 60%, at least 70%, at least 80%, at least 90%, at least 95%, at least 96%, at least 97%, at least 98% or at least 99% of the injection formulation into the xylem and/or phloem of the plant. In some variations of the foregoing, the methods deliver the injection formulation into to the active vasculature of the plant in an average maximum time of less than 10 minutes, or less than 5 minutes.

In certain embodiments, the method comprises injecting injection formulation into the vasculature through one or more sites on the trunk or stem of the plant. In embodiments where the formulation is injected through multiple injection sites, a plurality of the injection systems described herein may be used. In some embodiments where the formulation is injected through multiple injection sites, the system comprises multiple injection tools operatively connected to a single fluid delivery unit. In some variations, the method further comprises removing at least a portion of the bark around the injection site, e.g., prior to piercing the trunk.

The methods described herein generally provide one or more commercial advantages over the methods currently known in the art to control citrus greening disease. For example, advantages include one or more of a faster return to the production yields pre-infection, fast response (e.g., curing), lower volumes of formulation needed, less loss of formulation to the environment, less damage to the plant, response in old plants, response in plants with significant disease symptoms.

### ii) Injection Technology Applied to Treat Citrus Greening (HLB)

In some aspects, provided herein are methods for controlling citrus greening disease in a citrus plant using compositions and formulations as described herein. In some embodiments, citrus greening disease in these citrus plants are controlled by precisely injecting a liquid formulation comprising at least one antimicrobial peptide into the active vasculature of the citrus plant. In some variations, the liquid formulation is injected no further than the active vasculature of the citrus plant.

There are multiple advantages to using antimicrobial peptides for treating bacterial diseases, e.g., those bacteria that cause HLB. Many of the current solutions for treating bacterial diseases in agriculture, including HLB, rely on the use of antibiotics, where resistance has developed or is likely to develop. Additionally, with the known problems associated with widespread antibiotic usage many countries are phasing out the use of antibiotics to treat certain agriculturally relevant bacterial diseases. Antimicrobial peptides are less likely to induce antimicrobial resistance. Moreover, the antimicrobial peptides disclosed herein are short linear antibacterial peptides and thus, should readily degrade in the environment providing an added benefit over traditional antibiotics that can accumulate in the environment.

In some embodiments, the method comprises injecting the citrus plant with an injection formulation described herein. In some embodiments, an antimicrobial peptide solution is delivered to citrus trees using injection technology to deliver the peptides directly to the vasculature of the tree. In some embodiments, the formulation comprising at least one antimicrobial peptides is injected in a tree with the Trecise injection technology, as disclosed in WO2020021041, which is incorporated in its entirety herein. In some embodiments, the direct application of the formulation comprising an antimicrobial peptide to the vasculature of the tree, where CLas resides, increases the likelihood the peptides will come into direct contact with the CLas pathogen.

In some embodiments, injecting the injection formulation or any of the methods described herein are performed 4 times a year. In some embodiments, injecting the injection formulation or any of the methods described herein are performed 1, 2, 3, 4, 5, 6, 7, 8, 9, 10, 11, or 12 times a year. In some embodiments, injecting the injection formulation or any of the methods described herein are performed more than 1 time, more than 2 times, more than 3 times, more than 4 times, more than 5 times, more than 6 times, more than 7 times, more than 8 times, more than 9 times, or more than 10 times a year. In some embodiments, injecting the injection formulation or any of the methods described herein are performed less than 2 times, less than 3 times, less than 4 times, less than 5 times, less than 6 times, less than 7 times, less than 8 times, less than 9 times, or less than 10 times a year.

In some embodiments, the citrus plant is a citrus tree or a citrus bush. In some variations, the citrus tree is an orange tree, a lemon tree, a lime tree, a grapefruit tree, or a pomelo tree. In some embodiments, the orange tree is a Citrus sinensis tree. In some embodiments, the orange tree is a Citrus x aurantium tree. In certain variations, the citrus plant is a lemon bush, or a lime bush. In some embodiments, the citrus plant is one of Citrus x macrocarpa, Citrus medica, Citrus x paradisi, Citrus japonica, Citrus limon, Citrus reticulata, Citrus maxima, or Citrus unshiu. In one variation, the citrus bush is a dwarf citrus bush. In other variations, the citrus tree is a mature tree. In some embodiments, the citrus tree is a fruit bearing tree. In some embodiments, the citrus tree is a non-bearing tree.

In some variations, the citrus plants are suffering from citrus greening disease caused by Liberibacter spp. (e.g., L. asiaticus, L. africanus, L. americanus). In some variations, the disease is transmitted by the Asian citrus psyllid, Diaphorina citri, and the African citrus psyllid, Trioza erytreae.

In some embodiments, the infected citrus plant exhibits at least one symptom caused by citrus greening disease. In some embodiments, the citrus plant to which the injection formulation is applied is infected. In some embodiments, the citrus plant to which the injection formulation is applied is not infected. In some embodiments, the methods described herein are used only for citrus plants with one or more symptoms caused by citrus greening disease. Such symptoms may include any one or more of the following: asymmetrical yellowing of veins and adjacent tissues; splotchy mottling of the entire leaf; premature defoliation; dieback of twigs; decay of feeder rootlets and lateral roots; decline in vigor; stunted growth, bear multiple off-season flowers; produce small, irregularly shaped fruit with a thick, pale peel that remains green at the bottom and tastes bitter.

In some variations, to assess the efficacy of the injection formulations used in the citrus plant, one or more of the following are evaluated: BRIX analysis of fruit, fruit yield, fruit drop, antimicrobial peptide residue levels in fruit, antimicrobial peptide concentrations in citrus leaves, effects of the treatment on CLas titers in leaves, and overall plant health.

BRIX, or Balling Relative Intensity Index, is a measure of sugar content of an aqueous solution. As described herein, BRIX may be measured by any number of common methods known in the art, such as using a refractometer. See, e.g., Jaywant, S. A.; Singh, H.; Arif, K. M. Sensors and Instruments for Brix Measurement: A Review. Sensors 2022, 22, 2290.

In some embodiments, this disclosure provides methods for enhancing or maintaining plant health in the citrus plants and grove. In some such embodiments, this disclosure provides methods for treating diseased plants and/or methods for controlling the bacteria, fungi, viruses and/or other pathogens that cause citrus greening disease in the citrus plants. In further such embodiments, this disclosure provides methods for treating citrus plants whose xylem and/or phloem have been invaded by disease-causing bacteria, fungi, viruses, and/or other pathogens, for controlling the bacteria, fungi, virus and/or other pathogens causing the disease, and for preventing diseases by preventing sufficient colonization of the plant by the disease-causing pathogens such as bacteria, fungi, and viruses.

In some embodiments, controlling citrus greening disease in citrus plants using the systems, devices and methods herein includes reducing the bacterial concentration (titer) in the vascular system. In some variations, controlling citrus greening disease in citrus plants using the systems, devices and methods herein includes reducing the bacterial concentration (titer) in the vascular system by strengthening the plant's natural defense system. In certain embodiments, the systems, devices and methods herein can provide a treatment that leads to suppression of the disease to a level where recovery of citrus production occurs. In some variations, bacterial titer refers to the bacterial concentration in the vascular system of the infected plant. Bacterial titer may be measured using any suitable methods and techniques known in the art. For example, in one variation, bacterial titer is measured through quantitative PCR. In one variation, CLas titer is measured, e.g., using any suitable techniques known in the art.

In some variations, the treatment protocols provided herein can (i) reduce fruit drop; (ii) increase Brix in the fruit; and/or (iii) increase fruit yield. In certain variations, the treatment protocols provided herein can (i) reduce fruit drop by at least 10%, at least 15%, at least 20%, or at least 25%, or between 5% and 50%, between 5% and 40%, between 10% and 30%, or between 15% and 25%; (ii) increase Brix by at least 1%, or at least 5%, or between 1% and 10%; and/or (iii) increase yield by at least 25%, at least 30%, at least 35%, at least 40%, at least 45%, or at least 50%, or between 25% and 75%, or between 40% and 60%. Overall, in one variation, the treatment protocols provided herein can improve recovery of plant health, and yield a healthier, more resilient grove.

In some variations, the average fruit drop for the plants to which the injection formulation is administered is less than 25, less than 20, less than 15 or less than 10; or between 10 and 25. In some variations, the average fruit yield for the plants to which the injection formulation is administered is at least 35 lbs, at least 40 lbs, at least 45 lbs, at least 50 lbs, at least 55 lbs, at least 60 lbs, at least 65 lbs, at least 70 lbs, at least 75 lbs, at least 80 lbs, or least 85 lbs per plant, or between 30 and 90 lbs, or between 35 and 85 lbs per plant.

In some variations, an average Brix for the plants to which the injection formulation is administered is at least 7.5, at least 8, or at least 8.5; or between 7 and 9, or between 7.5 and 8.5.

In other embodiments, controlling citrus greening disease in citrus plants using the systems, devices and methods herein includes at least partially or fully restoring phloem functionality of the infected citrus plants. In certain embodiments of the foregoing, this may restore the plant's productive capacity and overall plant health including the metabolomic profile of the plant. In some variations, metabolomic profile of the plant may be used to measure the plant health. In yet other embodiments, controlling citrus greening disease in citrus plants using the systems, devices and methods herein includes at least partially or fully restoring yield capacity. In some variations, yield over the plant lifecycle is increased as compared to untreated control plants.

### 6. Method of Increasing Plant Fitness

In one aspect, provided herein is a method of increasing the fitness of a plant, the method including delivering to the plant the antimicrobial peptide composition described herein (e.g., in an effective amount and duration) to increase the fitness of the plant relative to an untreated plant (e.g., a plant that has not been delivered the antimicrobial peptide composition).

In some embodiments the plant fitness is increased by treating the plant with a composition comprising an effective amount of at least one antimicrobial peptide, antimicrobial peptide precursor, antimicrobial peptide fragment, or antimicrobial peptide motif, and that includes (a) an amino acid sequence of an antimicrobial peptide that has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity with a sequence selected from the group consisting of SEQ ID NO: 1-303 and 326-374 and 401-420; or (b) an amino acid sequence of an antimicrobial peptide motif; and a carrier. In some embodiments of this aspect, the antimicrobial peptide motif includes at least one of SEQ ID NO: 1-303 of 326-374 or 401-420. In additional embodiments of this aspect, which may be combined with any preceding embodiment, the antimicrobial peptide is active and/or toxic towards microbial cells. In some embodiments, the antimicrobial peptide is not toxic towards mammalian cells, e.g., human cells.

An increase in the fitness of the plant as a consequence of delivery of an antimicrobial peptide composition can manifest in a number of ways, e.g., thereby resulting in a better production of the plant, for example, an improved yield, improved vigor of the plant (e.g., improved tolerance of abiotic or biotic stress or improved resistance to pests) or improved quality of the harvested product from the plant. An improved yield of a plant relates to an increase in the yield of a product (e.g., as measured by plant biomass, grain, seed or fruit yield, protein content, carbohydrate or oil content or leaf area) of the plant by a measurable amount over the yield of the same product of the plant produced under the same conditions, but without the application of the instant compositions or compared with application of conventional agricultural agents. For example, yield can be increased by at least about 0.5%, about 1%, about 2%, about 3%, about 4%, about 5%, about 10%, about 20%, about 30%, about 40%, about 50%, about 60%, about 70%, about 80%, about 90%, about 100%, or more than 100%. Yield can be expressed in terms of an amount by weight or volume of the plant or a product of the plant on some basis. The basis can be expressed in terms of time, growing area, weight of plants produced, or amount of a raw material used. For example, such methods may increase the yield of plant tissues including, but not limited to: seeds, fruits, kernels, bolls, tubers, roots, and leaves.

An increase in the fitness of a plant as a consequence of delivery of a antimicrobial peptide composition can also be measured by other methods, such as an increase or improvement of the vigor rating, the stand (the number of plants per unit of area), plant height, stalk circumference, stalk length, leaf number, leaf size, plant canopy, visual appearance (such as greener leaf color), root rating, emergence, protein content, increased tillering, bigger leaves, more leaves, less dead basal leaves, stronger tillers, less fertilizer needed, less seeds needed, more productive tillers, earlier flowering, early grain or seed maturity, less plant verse (lodging), increased shoot growth, earlier germination, or any combination of these factors, by a measurable or noticeable amount over the same factor of the plant produced under the same conditions, but without the administration of the instant compositions or with application of conventional agricultural agents.

Provided herein is a method of modifying or increasing the fitness of a plant, the method including delivering to the plant an effective amount of a antimicrobial peptide composition provided herein, wherein the method modifies the plant and thereby introduces or increases a beneficial trait in the plant (e.g., by about 1%, 2%, 5%, 10%, 20%, 30%, 40%, 50%, 60%, 70%, 80%, 90%, 100%, or more than 100%) relative to an untreated plant. In particular, the method may increase the fitness of the plant (e.g., by about 1%, 2%, 5%, 10%, 20%, 30%, 40%, 50%, 60%, 70%, 80%, 90%, 100%, or more than 100%) relative to an untreated plant.

In some instances, the increase in plant fitness is an increase (e.g., by about 1%, 2%, 5%, 10%, 20%, 30%, 40%, 50%, 60%, 70%, 80%, 90%, 100%, or more than 100%) in disease resistance, drought tolerance, heat tolerance, cold tolerance, salt tolerance, metal tolerance, herbicide tolerance, chemical tolerance, water use efficiency, nitrogen utilization, resistance to nitrogen stress, nitrogen fixation, pest resistance, herbivore resistance, pathogen resistance, yield, yield underwater-limited conditions, vigor, growth, photosynthetic capability, nutrition, protein content, carbohydrate content, oil content, biomass, shoot length, root length, root architecture, seed weight, or amount of harvestable produce.

In some instances, the increase in fitness is an increase (e.g., by about 1%, 2%, 5%, 10%, 20%, 30%, 40%, 50%, 60%, 70%, 80%, 90%, 100%, or more than 100%) in development, growth, yield, resistance to abiotic stressors, or resistance to biotic stressors. An abiotic stress refers to an environmental stress condition that a plant or a plant part is subjected to that includes, e.g., drought stress, salt stress, heat stress, cold stress, and low nutrient stress. A biotic stress refers to an environmental stress condition that a plant or plant part is subjected to that includes, e.g., nematode stress, insect herbivory stress, fungal pathogen stress, bacterial pathogen stress, or viral pathogen stress. The stress may be temporary, e.g., several hours, several days, several months, or permanent, e.g., for the life of the plant.

In some instances, the increase in plant fitness is an increase (e.g., by about 1%, 2%, 5%, 10%, 20%, 30%, 40%, 50%, 60%, 70%, 80%, 90%, 100%, or more than 100%) in quality of products harvested from the plant. For example, the increase in plant fitness may be an improvement in commercially favorable features (e.g., taste or appearance) of a product harvested from the plant. In other instances, the increase in plant fitness is an increase in shelf-life of a product harvested from the plant (e.g., by about 1%, 2%, 5%, 10%, 20%, 30%, 40%, 50%, 60%, 70%, 80%, 90%, 100%, or more than 100%).

Alternatively, the increase in fitness may be an alteration of a trait that is beneficial to human or animal health, such as a reduction in allergen production. For example, the increase in fitness may be a decrease (e.g., by about 1%, 2%, 5%, 10%, 20%, 30%, 40%, 50%, 60%, 70%, 80%, 90%, 100%, or more than 100%) in production of an allergen (e.g., pollen) that stimulates an immune response in an animal (e.g., human).

The modification of the plant (e.g., increase in fitness) may arise from modification of one or more plant parts. For example, the plant can be modified by contacting leaf, seed, pollen, root, fruit, shoot, flower, cells, protoplasts, or tissue (e.g., meristematic tissue) of the plant. As such, in another aspect, provided herein is a method of increasing the fitness of a plant, the method including contacting pollen of the plant with an effective amount of a antimicrobial peptide composition herein, wherein the method increases the fitness of the plant (e.g., by about 1%, 2%, 5%, 10%, 20%, 30%, 40%, 50%, 60%, 70%, 80%, 90%, 100%, or more than 100%) relative to an untreated plant.

In yet another aspect, provided herein is a method of increasing the fitness of a plant, the method including contacting a seed of the plant with an effective amount of a antimicrobial peptide composition disclosed herein, wherein the method increases the fitness of the plant (e.g., by about 1%, 2%, 5%, 10%, 20%, 30%, 40%, 50%, 60%, 70%, 80%, 90%, 100%, or more than 100%) relative to an untreated plant.

In another aspect, provided herein is a method including contacting a protoplast of the plant with an effective amount of a antimicrobial peptide composition herein, wherein the method increases the fitness of the plant (e.g., by about 1%, 2%, 5%, 10%, 20%, 30%, 40%, 50%, 60%, 70%, 80%, 90%, 100%, or more than 100%) relative to an untreated plant.

In a further aspect, provided herein is a method of increasing the fitness of a plant, the method including contacting a plant cell of the plant with an effective amount of a antimicrobial peptide composition herein, wherein the method increases the fitness of the plant (e.g., by about 1%, 2%, 5%, 10%, 20%, 30%, 40%, 50%, 60%, 70%, 80%, 90%, 100%, or more than 100%) relative to an untreated plant.

In another aspect, provided herein is a method of increasing the fitness of a plant, the method including contacting meristematic tissue of the plant with an effective amount of a antimicrobial peptide composition herein, wherein the method increases the fitness of the plant (e.g., by about 1%, 2%, 5%, 10%, 20%, 30%, 40%, 50%, 60%, 70%, 80%, 90%, 100%, or more than 100%) relative to an untreated plant.

In another aspect, provided herein is a method of increasing the fitness of a plant, the method including contacting an embryo of the plant with an effective amount of a antimicrobial peptide composition herein, wherein the method increases the fitness of the plant (e.g., by about 1%, 2%, 5%, 10%, 20%, 30%, 40%, 50%, 60%, 70%, 80%, 90%, 100%, or more than 100%) relative to an untreated plant.

In cases where an herbicide is included in the antimicrobial peptide composition, the methods may be further used to decrease the fitness of or kill weeds. In such instances, the method may be effective to decrease the fitness of the weed by about 2%, 5%, 10%, 20%, 30%, 40%, 50%, 60%, 70%, 80%, 90%, 100%, or more in comparison to an untreated weed (e.g., a weed to which the antimicrobial peptide composition has not been administered). For example, the method may be effective to kill the weed, thereby decreasing a population of the weed by about 2%, 5%, 10%, 20%, 30%, 40%, 50%, 60%, 70%, 80%, 90%, 100%, or more in comparison to an untreated weed. In some instances, the method substantially eliminates the weed. Examples of weeds that can be treated in accordance with the present methods are further described herein.

### 7. Methods of Plant Transformation

Additional embodiments are directed to the generation of transgenic plants expressing one or more of the antimicrobial peptides disclosed herein that provide resistance against infection by gram-negative bacterial pathogens. In some embodiments, the transgenic plant is a transgenic HLB-resistant citrus plants that expresses one or more of the antimicrobial peptides of the invention.

In some embodiments, the plant is transformed with constructs encoding at least one antimicrobial peptide, antimicrobial peptide precursor, antimicrobial peptide fragment, or antimicrobial peptide motif, and that includes (a) an amino acid sequence of an antimicrobial peptide that has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity with a sequence selected from the group consisting of SEQ ID NO: 1-303 and 326-374 and 401-420; or (b) an amino acid sequence of an antimicrobial peptide motif; and a carrier. In some embodiments of this aspect, the antimicrobial peptide motif includes at least one of SEQ ID NO: 1-303 or 326-374 or 401-420. In additional embodiments of this aspect, which may be combined with any preceding embodiment, the antimicrobial peptide is active and/or toxic towards microbial cells. In some embodiments, the antimicrobial peptide is not toxic towards mammalian cells, e.g., human cells.

Furthermore, the construct encoding any of the preceding embodiments' sequence can be designed so that it will be optimally expressed in the organism in which the construct will be expressed. For example, the construct can be codon-optimized for expression in, e.g., in E. coli cells, or for expression in e.g., plants. U.S. Pat. No. 5,500,365 describes a method for synthesizing plant genes to optimize the expression level of the protein encoded by the synthesized gene. This method relates to the modification of the structural gene sequences of the exogenous recombinant or edited polynucleotide, to make them more “plant-like” and therefore more efficiently transcribed, processed, translated, and expressed by the plant. Features of genes that are expressed well in plants include use of codons that are commonly used by the plant host and elimination of sequences that can cause undesired intron splicing or polyadenylation in the coding region of a gene transcript. A similar method for obtaining enhanced expression of transgenes in monocotyledonous plants is disclosed in U.S. Pat. No. 5,689,052, which is incorporated in its entirety herein.

Examples of methods for inserting foreign DNA into the plant genome with clustered regularly interspaced short palindromic repeats (CRISPR)-associated (Cas)-guide RNA technology and a Cas endonuclease are at least disclosed by Svitashev et al., 2015; Murovec et al., 2017; Kumar and Jain, 2015; and in US Patent Appl. Pub. No. 20150082478, which is specifically incorporated herein by reference in its entirety.

Further provided are methods for modifying insects, mollusks, fungi, and nematodes by providing for consumption a plant comprising a recombinant polynucleotide e.g., recombinant ssRNAs, e.g., recombinant ssRNA vectors) comprising one or more sequences of or derived from a viroid and one or more heterologous effector sequences, e.g., an antimicrobial peptide, which have a biological effect on an organism. Methods for modifying plants by delivery of such recombinant viroid polynucleotides are described in WO2022020378A1, which is disclosed in its entirety herein.

### 8. Methods of Treating a Fungal Disease in a Subject

A further aspect of the disclosure relates to methods of treating a subject with a fungal disease including administering to a subject an antifungal or fungicidal composition including the antimicrobial peptide, the antimicrobial peptide precursor, the antimicrobial peptide fragment, or the antimicrobial peptide motif of any of the preceding embodiments and a pharmaceutically acceptable carrier. In some embodiments of this aspect, the subject is a mammal; in other embodiments the subject is a vertebrate such as a bird, reptile, fish, or amphibian, or is an invertebrate such as an insect. In additional embodiments of this aspect, the mammal is a human. In further embodiments of this aspect, the mammal is a domestic animal or livestock. In still further embodiments of this aspect, which may be combined with any preceding embodiment, the fungal disease is caused by a fungal pathogen selected from the group of Aspergillus, Candida, Coccidioides, Histoplasma, Cryptococcus, Pneumocystis, or Blastomyces fungus. In some embodiments, the fungal disease is an infection of a Mucoromycotina fungus, a Candida species (e.g., C. albicans, C. auris, C. tropicalis, C. krusei, C. glabrata, C. parapsilosis, and C. pseudotropicalis), a Coccidioides species (e.g., C, immitis or C. posadasii), an Aspergillus species (e.g., A. fumigatus, A. flavus, and A. niger), a Mucor species, a Rhizomucor species, a Malassezia species (e.g., M. furfur, M. globose, and M. restricta), Magnaporthe oryzae, Botrytis cinerea, Puccinia spp., Fusarium graminearum; Fusarium oxysporum, Blumeria graminis, Mycosphaerella graminicola, Colletotrichum spp., Ustilago maydis; Melampsora lini, Phakopsora pachyrhizi, or Rhizoctonia solani. In yet another embodiment of this aspect, which may be combined with any preceding embodiment, the fungal disease is aspergillosis, blastomycosis, candidiasis, coccidioidomycosis, histoplasmosis, mucormycosis, mycetoma, ringworm, sporotrichosis, paracoccidioidomycosis, talaromycosis, chromoblastomycosis fusariosis, emergomycosis, scedosporiosis, or fungal meningitis. In additional embodiments of this aspect, which may be combined with any preceding embodiments, the antifungal or fungicidal composition is administered intravenously, intramuscularly, subcutaneously, topically, orally, transdermally, intraperitoneally, intraorbitally, by implantation, by inhalation, intrathecally, intraventricularly, or intranasally.

As used herein, the term “subject” refers to an organism, such as an animal, plant, or microbe. In some embodiments, the subject is a mammal. In some embodiments, the mammal is a human. In other embodiments, the subject is a domestic animal or livestock. In some embodiments, the subject is a non-mammal. In some embodiments, the non-mammal is a reptile, an insect, an amphibian, a bird, or a fish. In embodiments, the subject is a vertebrate animal (e.g., mammal, bird, cartilaginous or bony fish, reptile, or amphibian). In embodiments, the subject is a human; including adults and non-adults (infants and children). In embodiments, the subject is a non-human mammal, such as a non-human primate (e.g., monkeys, apes), ungulate (e.g., cattle, buffalo, bison, sheep, goat, pig, camel, llama, alpaca, deer, horses, donkeys), carnivore (e.g., dog, cat), rodent (e.g., rat, mouse), or lagomorph (e.g., rabbit). In embodiments, the subject is a bird, such as a member of the avian taxa Galliformes (e.g., chickens, turkeys, pheasants, quail), Anseriformes (e.g., ducks, geese), Paleaognathae (e.g., ostriches, emus), Columbiformes (e.g., pigeons, doves), or Psittaciformes (e.g., parrots). In embodiments, the subject is an invertebrate such as an arthropod (e.g, insects, arachnids, crustaceans), a nematode, an annelid, a helminth, or a mollusc. In embodiments, the subject is an organism that is part of a symbiosis, such as part of the microbiome of an animal or a plant. In embodiments, the subject is a plant, such as an angiosperm plant (which can be a dicot or a monocot) or a gymnosperm plant (e.g., a conifer, a cycad, a gnetophyte, a Ginkgo), a fern, horsetail, clubmoss, or a bryophyte. In embodiments, the subject is a eukaryotic alga (unicellular or multicellular). In embodiments, the subject is a plant of agricultural or horticultural importance, such as row crop plants, fruit-producing plants and trees, vegetables, trees, and ornamental plants including ornamental flowers, shrubs, trees, groundcovers, and turf grasses. Plants and plant cells are of any species of interest, including dicots and monocots. Plants of interest include row crop plants, fruit-producing plants and trees, vegetables, trees, and ornamental plants including ornamental flowers, shrubs, trees, groundcovers, and turf grasses.

The inventive concepts that can be embodied in a wide variety of specific contexts. The specific embodiments discussed herein are merely illustrative of specific ways to make and use the invention and do not delimit the scope of the invention.

### 9. Other Methods

In some embodiments, any of the antimicrobial peptide, antimicrobial peptide precursor, antimicrobial peptide fragment, or antimicrobial peptide motif encoded by SEQ ID NO: 1-260 or 401-414 is active and/or toxic to a structural element fungal pathogen (e.g., a fungal pathogen that infests or damages human-built structures such as buildings or other human-created artifacts, or components thereof). In some embodiments, the antimicrobial peptide, antimicrobial peptide precursor, antimicrobial peptide fragment, or antimicrobial peptide motif inhibits growth or reproduction of or is toxic to a fungus that damages wood or other materials useful in human-built structures or artifacts; examples include wood-decaying fungi that cause brown rot, white rot, or soft rot, or fungi that cause dry rot in human-built structures or buildings. In some embodiments, the structural element fungal pathogen is dry rot fungus (Serpula lacrymans), cellar rot fungus (Coniphora puteana), a wet rot fungus (Antrodia vaillantii, A. xantha, Asterostroma spp., Donkioporia expansa, Paxillus panuoides, Phellinus contignuus, Tyromyces placentus), or a fungus that colonizes water-damaged structural materials, e.g., Penicillium chrysogenum, Aspergillus versicolor, Chaetomium spp., Acremonium spp., Ulocladium spp., Stachybotrys spp., Arthrinium phaeospermum, Aureobasidium pullulans, Cladosporium herbarum, Trichoderma spp., Aspergillus fumigatus, Aspergillus melleus, Aspergillus niger, Aspergillus ochraceus, Mucor racemosus, or Mucor spinosus.

### Examples

### Example 1. Design of Alpha-Factor Variant Peptides

Antimicrobial alpha-factor peptides were designed with enhanced antifungal activity towards Botrytis.

### Experimental Procedure:

### Design of Alpha Factor Variants:

### 1) Based on Six Models of the Botrytis STE2 GPCR

Six models of the Botrytis STE2 GPCR were used in the design of antimicrobial peptides with improved activity. One model was directly from the AlphaFold 2 database, and five other models were built using the 5 yeast STE2 structures available in the PDB (7QA8, 7AD3, 7QB9, 7QBC and 7QBI). These models were built with the natural yeast alpha-factor peptide (13 amino acids long) bound in place and further refined.

The best variants were prioritized for screening using several biochemical filters such as synthesizability, hydrophobicity, and instability index. Tables 3 depicts the list of peptides designed, of which the peptides corresponding to SEQ ID 1-180 were tested in biological assays (see Example 2). Table 4 depicts designed homo- and hetero dimers comprising antimicrobial peptide domains that are linked together with a linker (e.g., GGGG (SEQ ID NO: 376)), and constructs comprising additional peptide domains (e.g., a cell penetrating peptide domain).

TABLE 3
Alpha factor variant peptides

| SEQ ID | amino acid sequence |
|---|---|
| 1 | WHWLQFWRGQSMY |
| 2 | PHWGSFKKGQPMY |
| 3 | KHWVTLKWGAPMY |
| 4 | WHLLRFKPGQPMY |
| 5 | PHWLRFKPGQPMY |
| 6 | PHWVQLKRGAPMY |
| 7 | KHWLQIKYGEPMY |
| 8 | KHWIKLMPGQGMY |
| 9 | KHWLRFKPGQPMY |
| 10 | PHWVQIKLGQPMY |
| 11 | KHWITFKPGQPMY |
| 12 | KHVLRFKPGQPMY |
| 13 | KHWLKFKPGQPMY |
| 14 | KHWLKLWPGQPMY |
| 15 | WHTLKIKKGEPMY |
| 16 | WHWLTFKTGQGMY |
| 17 | KHFLKLWPGQPMY |
| 18 | KHFLRFKPGQPMY |
| 19 | PKWIRLKPGQPMY |
| 20 | KHWLSFKPGQPMY |
| 21 | KHWVQLMKGQPMY |
| 22 | KHLVQFKMGQPMY |
| 23 | WHWVALKNGQGMY |
| 24 | KHYLNFKPGQPMY |
| 25 | WHLLQFKVGQPMY |
| 26 | WHWIQFKPGQSMY |
| 27 | KHFLNLKSGQPMY |
| 28 | KHFLALKPGQPMY |
| 29 | KHDLRFKPGQPMY |
| 30 | PHWLTIKPGQPMY |
| 31 | KEWIQLKLGQPMY |
| 32 | KSWLSLKPGQGMY |
| 33 | WRWGQFKPGEPMY |
| 34 | PHWLSFFPGQGMY |
| 35 | KCWLELKTGMPMY |
| 36 | WHMLEFKPGQPMY |
| 37 | PHWIQLFNGQPMY |
| 38 | KHVLQFKPGEPMY |
| 39 | KSWGQFKPGQPMY |
| 40 | PEWIQLKTGMPMY |
| 41 | PHLLNFKPGQPMY |
| 42 | PHSGQLWSGQPMY |
| 43 | PHWGQFKLGQPMY |
| 44 | PHWVNLKTGQGMY |
| 45 | PTGLQFKPGQSMY |
| 46 | WHWLSLNTGQPMY |
| 47 | KHDLELWPGQPMY |
| 48 | WGGLALKPGQPMY |
| 49 | PEWLKLYPGQPMY |
| 50 | WRVLQLKPGSSMY |
| 51 | WHWVRIWPGQPMY |
| 52 | KHWLRIKWGEPMY |
| 53 | WKWVRLKTGQPMY |
| 54 | KHMIQLYWGQPMY |
| 55 | WRYLRLKPGSGMY |
| 56 | WHSLKLKRGQPMY |
| 57 | WHWLAIFRGQPMY |
| 58 | WKWVQIKKGAPMY |
| 59 | KHWITLKSGMPMY |
| 60 | WKTLQLKIGAPMY |
| 61 | KHWLKLFPGQPMY |
| 62 | KHWLKIKIGEPMY |
| 63 | PRWLQIKKGQSMY |
| 64 | KHWLQFKPGSGMY |
| 65 | WKWVNLRTGQPMY |
| 66 | PHWISLKKGQPMY |
| 67 | KHMLRIKPGQPMY |
| 68 | KHWGRLRAGQPMY |
| 69 | KHWLRLNPGQPMY |
| 70 | WHYLRLKPGQSMY |
| 71 | WHWIQLKKGEPMY |
| 72 | KHYLKFKPGQPMY |
| 73 | WQWLRIKSGQGMY |
| 74 | WHFVKLHPGQPMY |
| 75 | KHWIRLWPGQPMY |
| 76 | WHWLRLKPGASMY |
| 77 | PHWIKLHTGQPMY |
| 78 | KHFLNFKPGQPMY |
| 79 | WRDIRLKKGQPMY |
| 80 | KHTLRFKPGQPMY |
| 81 | KHWIKLKNGQPMY |
| 82 | WHWGSLKVGQPMY |
| 83 | KHTLRLKPGQPMY |
| 84 | KHMLRFKPGQPMY |
| 85 | WHFLKFKPGEPMY |
| 86 | KHFLKIKPGQPMY |
| 87 | KHVLKFKPGQPMY |
| 88 | KHLLRLKPGQPMY |
| 89 | WHSLEFKIGQGMY |
| 90 | WHFLQIKPGQGMY |
| 91 | KQWLKLKNGQPMY |
| 92 | WHTLRFKPGQPMY |
| 93 | KHTLQLKRGQPMY |
| 94 | KHYLSFKPGQPMY |
| 95 | KHLLTFKPGQPMY |
| 96 | KHFLTLKPGQPMY |
| 97 | WHWVQFRPGAPMY |
| 98 | WKWGSLKNGQPMY |
| 99 | WHWLKIWPGQPMY |
| 100 | WKYLNFKPGQPMY |
| 101 | WHVLRFKPGQPMY |
| 102 | KHYLTLKPGQPMY |
| 103 | KHYLALKPGQPMY |
| 104 | KRGINLKPGQPMY |
| 105 | WHILRFKNGMPMY |
| 106 | WHWVQLKKGEPMY |
| 107 | KHFLSLKPGQPMY |
| 108 | WHWLKLNYGQPMY |
| 109 | KRWLQIRPGEPMY |
| 110 | WHWLSIKPGQGMY |
| 111 | WHWVQFGPGSPMY |
| 112 | PHFLRLKPGQPMY |
| 113 | KHLLNLKPGQPMY |
| 114 | WHWINLWPGQPMY |
| 115 | KKVLQLKLGQPMY |
| 116 | KHDLKFKPGQPMY |
| 117 | WQSGQLKPGQPMY |
| 118 | WHMLNFKPGQPMY |
| 119 | WHWLKLGTGEPMY |
| 120 | WSMGQIKPGSPMY |
| 121 | PSGLQIKVGQPMY |
| 122 | PEWLSLKPGSSMY |
| 123 | KQWVQLKYGQGMY |
| 124 | WHGIQLFKGQPMY |
| 125 | KEWVQLYPGQPMY |
| 126 | PHDLEFKPGQGMY |
| 127 | WKSINFKPGQPMY |
| 128 | PHWVEFDPGQPMY |
| 129 | WQGLELHPGQPMY |
| 130 | WEVLTLKPGSPMY |
| 131 | PHLIQLGLGQPMY |
| 132 | PHMVRLKPGQPMY |
| 133 | KGWGQLIPGEPMY |
| 134 | WGDLKLKPGQPMY |
| 135 | WHWGQLQRGEPMY |
| 136 | PHWLKLKPGEPMY |
| 137 | WHMGQLKPGEPMY |
| 138 | WQMLKLKYGQPMY |
| 139 | PHWGEIKPGQSMY |
| 140 | KCFIQLKPGQPMY |
| 141 | PRDLTLKRGQPMY |
| 142 | PHGLSIYPGQPMY |
| 143 | PHWGELKPGQPMY |
| 144 | KHWVQLFPGQPMY |
| 145 | KEWLNFKNGQPMY |
| 146 | WRTGQFWPGQPMY |
| 147 | PHDGQLKRGQPMY |
| 148 | PKWLNLKPGEPMY |
| 149 | WRVLKLMPGQPMY |
| 150 | WHFLQIKPGEPMY |
| 151 | KEWLNLKPGQPMY |
| 152 | KSWLQIKNGQPMY |
| 153 | KGILTLKPGQPMY |
| 154 | KHVLALKPGSPMY |
| 155 | WKILTIKPGQPMY |
| 156 | KHDVQLKNGSPMY |
| 157 | WHWGNFQTGQPMY |
| 158 | PHWLNLKPGEPMY |
| 159 | WKSLNFKPGQSMY |
| 160 | PHWVSLKPGQPMY |
| 161 | PHWVAFKPGSPMY |
| 162 | KHYLELKPGQPMY |
| 163 | WHMVQLKPGEPMY |
| 164 | KGTLSLKPGQPMY |
| 165 | KHSLEFKPGQPMY |
| 166 | WEFVQFKRGQPMY |
| 167 | WHWLEFQRGQPMY |
| 168 | KHLVQLHPGQPMY |
| 169 | PHTIQLWKGQPMY |
| 170 | WHMLTFKPGQPMY |
| 171 | WHLGQFKPGEPMY |
| 172 | WSWGALKSGMPMY |
| 173 | PHWVQLKNGQPMY |
| 174 | WHSVQLHPGQPMY |
| 175 | KKWIQLKPGEPMY |
| 176 | WHDVNLKRGSPMY |
| 177 | KHTLEFKPGQPMY |
| 178 | WHSVQFWPGQPMY |
| 179 | KHFLELKPGQPMY |
| 180 | KHDLALKIGQPMY |
| 181 | KCWLNLKAGQGMY |
| 182 | KEWLQLNWGQPMY |
| 183 | KHLLNFKPGQPMY |
| 184 | KHMLKLKIGEPMY |
| 185 | KHMLQFSMGQPMY |
| 186 | KHMLQLKPGAGMY |
| 187 | KHVLRFKPGQPMY |
| 188 | KHWIKLKPGQPMY |
| 189 | KHWIQIKPGQSMY |
| 190 | KHWLQFNMGAPMY |
| 191 | KHWVQLNPGQPMY |
| 192 | KQYLQIKVGQPMY |
| 193 | KSWLELKIGQPMY |
| 194 | KSWVQLKAGEPMY |
| 195 | KTWLQLNLGEPMY |
| 196 | PHLLEFKPGQPMY |
| 197 | PHLLQFKVGEPMY |
| 198 | PHMLQIKYGQGMY |
| 199 | PHSLQFKPGQGMY |
| 200 | PHWIQFKKGQPMY |
| 201 | PHWIQLFAGQPMY |
| 202 | PHWIQLSLGQPMY |
| 203 | PHWLQFGKGQPMY |
| 204 | PHWLSIYWGQPMY |
| 205 | PHWLTLNYGMPMY |
| 206 | WCVLQIKWGMPMY |
| 207 | WCWGQLKIGEPMY |
| 208 | WCWLNLYNGQPMY |
| 209 | WCWVQFKPGQSMY |
| 210 | WEWIALNPGMPMY |
| 211 | WEWLELKLGQSMY |
| 212 | WGVLQFKLGQPMY |
| 213 | WGWIQLWRGQPMY |
| 214 | WGWLKFKYGMPMY |
| 215 | WGYLRLGYGQPMY |
| 216 | WGYLTIKLGQPMY |
| 217 | WHFLKLETGQPMY |
| 218 | WHFLSLKLGQPMY |
| 219 | WHGLALYSGQPMY |
| 220 | WHLIQIKLGQPMY |
| 221 | WHLVQFWPGQPMY |
| 222 | WHLVSIKWGQPMY |
| 223 | WHTLSLKWGQPMY |
| 224 | WHVGKLKAGSPMY |
| 225 | WHVLRIKYGQPMY |
| 226 | WHWGELWRGAPMY |
| 227 | WHWVQLNTGQPMY |
| 228 | WHWVTFKPGQSMY |
| 229 | WKFLQLKYGQPMY |
| 230 | WKFVQLKYGQSMY |
| 231 | WKLLQIKMGQPMY |
| 232 | WKLVQFKPGQPMY |
| 233 | WKWLALKIGQSMY |
| 234 | WKWLQFNWGQGMY |
| 235 | WKYLNLKPGQPMY |
| 236 | WKYVQLKIGQPMY |
| 237 | WQWVALNLGQPMY |
| 238 | WQWVQIKPGQSMY |
| 239 | WQYIQLKLGMPMY |
| 240 | WRLIQIKYGQPMY |
| 241 | WRLLQLHMGQSMY |
| 242 | WRLLQLKWGQPMY |
| 243 | WRSLELKIGQPMY |
| 244 | WRVLQFKTGEPMY |
| 245 | WRWVQFKIGQGMY |
| 246 | WSFLKLWTGQPMY |
| 247 | WSFLQFNPGQPMY |
| 248 | WSFLRLKPGSPMY |
| 249 | WSTLRFKVGQPMY |
| 250 | WSWLELKLGQSMY |
| 251 | WSWVELKPGMPMY |
| 252 | WSWVQLARGMPMY |
| 253 | WTDLQLFKGQPMY |
| 254 | WTILQLKPGAPMY |
| 255 | WTLIQLRLGQPMY |
| 256 | WTLLQLWRGQPMY |
| 257 | WTTLQLKPGSPMY |
| 258 | WTWLKLSPGQGMY |
| 259 | WTWLTFKPGMPMY |
| 260 | WTWVQLKPGASMY |
| 401 | WHWLQLKPGQPMY |
| 402 | KHILKFKPGQPMY |
| 403 | KHYLRFKPGQPMY |
| 404 | KHSLRLWPGQPMY |
| 405 | KHWLTLFPGQPMY |
| 406 | WQWLQLDRGQPMY |
| 407 | WHSLQIKKGQPMY |
| 408 | WHLLQFNWGQPMY |
| 409 | WTVLTLKRGQSMY |
| 410 | KHWLRFRPGQPMY |
| 411 | KHWLRFWPGQPMY |
| 412 | KHWLRIWPGQPMY |
| 413 | KHWLRLWPGQPMY |
| 414 | WHWLRLWPGQPMY |

TABLE 4
Alpha factor dimers and hybrid constructs

| SEQ ID | amin acid sequence |
|---|---|
| 261 | KHWGRLRAGQPMYGGGGKHWGRLRAGQPMY |
| 262 | KHWGRLRAGQPMYGGGGRRRRRRRR |
| 263 | KHWGRLRAGQPMYGGGGKKKKKKKK |
| 264 | KHWGRLRAGQPMYGGGGYMPQGARLRGWHK |
| 265 | YMPQGARLRGWHKGGGGKHWGRLRAGQPMY |
| 266 | KHFLRFKPGQPMYGSGSKHFLRFKPGQPMY |
| 267 | KHILKFKPGQPMYGGGGKHILKFKPGQPMY |
| 268 | KHILKFKPGQPMYGSGSKHILKFKPGQPMY |
| 269 | KHILKFKPGQPMYGSGSKHYLRFKPGQPMY |
| 270 | KHILKFKPGQPMYGSGSWTVLTLKRGQSMY |
| 271 | KHVLRFKPGQPMYGSGSKHVLRFKPGQPMY |
| 272 | KHWIKLKNGQPMYGSGSKHWIKLKNGQPMY |
| 273 | KHWLKFKPGQPMYGSGSKHWLKFKPGQPMY |
| 274 | KHWLKIKIGEPMYGSGSKHWLKIKIGEPMY |
| 275 | KHWLRFKPGQPMYGSGSKHWLRFKPGQPMY |
| 276 | KHWLRFRPGQPMYGSGSKHWLRFRPGQPMY |
| 277 | KHWLRIKWGEPMYGSGSKHWLRIKWGEPMY |
| 278 | KHYLKFKPGQPMYGSGSKHYLKFKPGQPMY |
| 279 | KHYLRFKPGQPMYGGGGKHYLRFKPGQPMY |
| 280 | KHYLRFKPGQPMYGSGSKHILKFKPGQPMY |
| 281 | KHYLRFKPGQPMYGSGSKHYLRFKPGQPMY |
| 282 | KHYLRFKPGQPMYGSGSWTVLTLKRGQSMY |
| 283 | PHWISLKKGQPMYGSGSPHWISLKKGQPMY |
| 284 | WTVLTLKRGQSMYGGGGWTVLTLKRGQSMY |
| 285 | WTVLTLKRGQSMYGSGSKHILKFKPGQPMY |
| 286 | WTVLTLKRGQSMYGSGSKHYLRFKPGQPMY |
| 287 | WTVLTLKRGQSMYGSGSWTVLTLKRGQSMY |
| 288 | YMPEGIKIKLWHKGSGSKHWLKIKIGEPMY |
| 289 | YMPEGWKIRLWHKGSGSKHWLRIKWGEPMY |
| 290 | YMPQGKKLSIWHPGSGSPHWISLKKGQPMY |
| 291 | YMPQGNKLKIWHKGSGSKHWIKLKNGQPMY |
| 292 | YMPQGPKFKLIHKGGGGKHILKFKPGQPMY |
| 293 | YMPQGPKFKLIHKGSGSKHILKFKPGQPMY |
| 294 | YMPQGPKFKLWHKGSGSKHWLKFKPGQPMY |
| 295 | YMPQGPKFKLYHKGSGSKHYLKFKPGQPMY |
| 296 | YMPQGPKFRLFHKGSGSKHFLRFKPGQPMY |
| 297 | YMPQGPKFRLVHKGSGSKHVLRFKPGQPMY |
| 298 | YMPQGPKFRLWHKGSGSKHWLRFKPGQPMY |
| 299 | YMPQGPKFRLYHKGGGGKHYLRFKPGQPMY |
| 300 | YMPQGPKFRLYHKGSGSKHYLRFKPGQPMY |
| 301 | YMPQGPRFRLWHKGSGSKHWLRFRPGQPMY |
| 302 | YMSQGRKLTLVTWGGGGWTVLTLKRGQSMY |
| 303 | YMSQGRKLTLVTWGSGSWTVLTLKRGQSMY |
| 326 | KCWLELKTGMPMYGGGGKCWLELKTGMPMY |
| 327 | KHFLRFKPGQPMYGGGGKHFLRFKPGQPMY |
| 328 | KHILKFKPGQPMYGGGGGEKEKEKEKEK |
| 329 | KHILKFKPGQPMYGGGGGRRRRRRRRRR |
| 330 | KHILKFKPGQPMYGGGGKHYLRFKPGQPMY |
| 331 | KHILKFKPGQPMYGGGGWTVLTLKRGQSMY |
| 332 | KHVLRFKPGQPMYGGGGKHVLRFKPGQPMY |
| 333 | KHWIKLKNGQPMYGGGGKHWIKLKNGQPMY |
| 334 | KHWLKFKPGQPMYGGGGKHWLKFKPGQPMY |
| 335 | KHWLKIKIGEPMYGGGGKHWLKIKIGEPMY |
| 336 | KHWLRFKPGQPMYGGGGKHWLRFKPGQPMY |
| 337 | KHWLRFRPGQPMYGGGGKHWLRFRPGQPMY |
| 338 | KHWLRFWPGQPMYGGGGKHWLRFWPGQPMY |
| 339 | KHWLRIKWGEPMYGGGGKHWLRIKWGEPMY |
| 340 | KHYLKFKPGQPMYGGGGKHYLKFKPGQPMY |
| 341 | KHYLRFKPGQPMYGGGGGEKEKEKEKEK |
| 342 | KHYLRFKPGQPMYGGGGGRRRRRRRRRR |
| 343 | KHYLRFKPGQPMYGGGGKHILKFKPGQPMY |
| 344 | KHYLRFKPGQPMYGGGGWTVLTLKRGQSMY |
| 345 | PHWISLKKGQPMYGGGGPHWISLKKGQPMY |
| 346 | PRWLQIKKGQSMYGGGGPRWLQIKKGQSMY |
| 347 | WHILRFKNGMPMYGGGGWHILRFKNGMPMY |
| 348 | WHWVQFGPGSPMYGGGGWHWVQFGPGSPMY |
| 349 | WQMLKLKYGQPMYGGGGWQMLKLKYGQPMY |
| 350 | WTVLTLKRGQSMYGGGGGEKEKEKEKEK |
| 351 | WTVLTLKRGQSMYGGGGGRRRRRRRRRR |
| 352 | WTVLTLKRGQSMYGGGGKHILKFKPGQPMY |
| 353 | WTVLTLKRGQSMYGGGGKHYLRFKPGQPMY |
| 354 | YMPEGIKIKLWHKGGGGKHWLKIKIGEPMY |
| 355 | YMPEGWKIRLWHKGGGGKHWLRIKWGEPMY |
| 356 | YMPMGNKFRLIHWGGGGWHILRFKNGMPMY |
| 357 | YMPMGTKLELWCKGGGGKCWLELKTGMPMY |
| 358 | YMPQGARLRGWHKGGGGKHWGRLRAGQPMY |
| 359 | YMPQGKKLSIWHPGGGGPHWISLKKGQPMY |
| 360 | YMPQGNKLKIWHKGGGGKHWIKLKNGQPMY |
| 361 | YMPQGPKFKLWHKGGGGKHWLKFKPGQPMY |
| 362 | YMPQGPKFKLYHKGGGGKHYLKFKPGQPMY |
| 363 | YMPQGPKFRLFHKGGGGKHFLRFKPGQPMY |
| 364 | YMPQGPKFRLVHKGGGGKHVLRFKPGQPMY |
| 365 | YMPQGPKFRLWHKGGGGKHWLRFKPGQPMY |
| 366 | YMPQGPRFRLWHKGGGGKHWLRFRPGQPMY |
| 367 | YMPQGPWFRLWHKGGGGKHWLRFWPGQPMY |
| 368 | YMPQGYKLKLMQWGGGGWQMLKLKYGQPMY |
| 369 | YMPSGPGFQVWHWGGGGWHWVQFGPGSPMY |
| 370 | YMSQGKKIQLWRPGGGGPRWLQIKKGQSMY |
| 371 | KHWGRLRAGQPMYEGEGGCKADGSWGVCCSGF |
| 372 | YMPQGARLRGWHKEGEGGCKADGSWGVCCSGF |
| 373 | FGSCCVGWSGDAKCGEGEGKHWGRLRAGQPMY |
| 374 | FGSCCVGWSGDAKCGEGEGYMPQGARLRGWHK |
| 413 | WHWLQLKPGQPMYYMPQGPKLQLWHW |
| 414 | YMPQGPKLQLWHWWHWLQLKPGQPMY |
| 415 | WHWLQLKPGQPMYGGGGYMPQGPKLQLWHW |
| 416 | YMPQGPKLQLWHWGGGGWHWLQLKPGQPMY |
| 417 | RRRRRRRRWHWLQLKPGQPMY |
| 418 | RRRRRRRRYMPQGPKLQLWHW |

### Example 2. Antifungal Activity Testing Towards Botrytis cinerea

The antifungal activity of 180 alpha-factor peptide variants towards Botrytis was tested. Experimental design:

### a) Peptide Synthesis

Peptides corresponding to SEQ ID NO: 1-180 were chemically synthesized using the solid-phase synthesis method and resuspended in water to a stock solution of 1 mM. The stock solution was aliquoted in cryovials and stored at −80° C.

### b) Botrytis Culturing

The Botrytis cinerea culture (B05.10) isolate was acquired from the Centraalbureau voor Schimmelcultures, the Netherlands. The Botrytis fungus was propagated on 0.5% V8 agar plates (200 ml V8 Juice, 3 g CaCO3, 15 g agar, 800-ml millipore water) at 20° C., in a 12 h photoperiod for 21 days.

Fungal conidia were harvested by scraping a Botrytis plate flooded with 1 ml water with 0.1% Tween 80 solution. Conidia were then collected in a 50 ml falcon tube by passing the solution through a 40 micron filter. The solution was then centrifuged at 6000 rpm and the pellet was resuspended in 1 ml of 20% glycerol. The conidia concentration of the resulting solution was counted using a hemocytometer. The stock was diluted to 107 spores/ml using 20% glycerol and frozen in 100 μl aliquots in −80° C. until further use.

c) Antifungal assay

Peptides were tested for activity against Botrytis, using the resazurin assay. For this, fungal stock conidia was first diluted to 5000 conidia/well. 50 μl of the spore suspension was added to each well of the sterile 96-well clear bottom testing plate and mixed with 50 μl of peptide solutions to a final concentration of 100, 50, 20, 15 and 10 μM. The plate was incubated for 24 h at 20° C. After 24 h, 10 μl of resazurin dye was added to the microtiter plate and incubated again for 20 h. Fluorescence was measured at 570/620 nm after 4 h of incubation with resazurin.

Growth inhibition was calculated by subtracting the fluorescence values of conidia only wells from sample wells and depicted as % inhibition of fungal growth. Four technical replicates were used for all treatments.

Results are shown in Table 5. Peptides that showed greater than 90% inhibition were considered active against Botrytis. Of the 180 peptides tested, 106 showed over 90% inhibition at 100 μM, and of those 78 showed >90% inhibition at 50 μM. The 78 peptides that were active at 50 μM were also tested at lower concentrations of 20 μM and 15 μM. Of those, 28 were active at 20 μM and of those, 8 peptides, with SEQ ID NOs 9, 12 13, 18, 52, 62, 68 and 72 were active at 15 μM. The peptide corresponding to SEQ ID NO: 68 was active at 10 μM. In summary, a selection of the designed alpha-factor variants showed a remarkable antifungal activity at concentrations as low as 10 to 15 μM.

TABLE 5
Antifungal activity of 180 alpha factor peptides towards Botrytis

| SEQ | Amino acid | Inhibition at¹ | | | | |
|---|---|---|---|---|---|---|
| ID NO: | sequence | 100 μM | 50 μM | 20 μM | 15 μM | 10 μM |
| 1 | WHWLQFWRGQSMY | 100% | 100% | <90% | n.d. | <90% |
| 2 | PHWGSFKKGQPMY | 100% | 100% | <90% | n.d. | <90% |
| 3 | KHWVTLKWGAPMY | 100% | 100% | 100.61% | <90% | <90% |
| 4 | WHLLRFKPGQPMY | 100% | 100% | <90% | n.d. | <90% |
| 5 | PHWLRFKPGQPMY | 100% | 100% | <90% | n.d. | <90% |
| 6 | PHWVQLKRGAPMY | 100% | 100% | <90% | n.d. | <90% |
| 7 | KHWLQIKYGEPMY | 100% | 100% | <90% | n.d. | <90% |
| 8 | KHWIKLMPGQGMY | 100% | 100% | <90% | n.d. | <90% |
| 9 | KHWLRFKPGQPMY | 100% | 100% | 100% | 100% | <90% |
| 10 | PHWVQIKLGQPMY | 100% | 100% | <90% | n.d. | <90% |
| 11 | KHWITFKPGQPMY | 100% | 100% | <90% | n.d. | <90% |
| 12 | KHVLRFKPGQPMY | 100% | 100% | 99.94% | 98.79% | <90% |
| 13 | KHWLKFKPGQPMY | 100% | 100% | 100% | 100% | <90% |
| 14 | KHWLKLWPGQPMY | 100% | 100% | <90% | n.d. | <90% |
| 15 | WHTLKIKKGEPMY | 100% | 100% | <90% | n.d. | <90% |
| 16 | WHWLTFKTGQGMY | 100% | 100% | <90% | n.d. | <90% |
| 17 | KHFLKLWPGQPMY | 100% | 100% | <90% | n.d. | <90% |
| 18 | KHFLRFKPGQPMY | 100% | 100% | 99.67% | 94.03% | <90% |
| 19 | PKWIRLKPGQPMY | 100% | 100% | <90% | n.d. | <90% |
| 20 | KHWLSFKPGQPMY | 100% | 100% | <90% | n.d. | <90% |
| 21 | KHWVQLMKGQPMY | 100% | 100% | <90% | n.d. | <90% |
| 22 | KHLVQFKMGQPMY | 100% | 100% | <90% | n.d. | <90% |
| 23 | WHWVALKNGQGMY | 100% | 98.68% | <90% | n.d. | <90% |
| 24 | KHYLNFKPGQPMY | 100% | 96.95% | <90% | n.d. | <90% |
| 25 | WHLLQFKVGQPMY | 100% | 92.63% | <90% | n.d. | <90% |
| 26 | WHWIQFKPGQSMY | 100% | 91.39% | <90% | n.d. | <90% |
| 27 | KHFLNLKSGQPMY | 100% | 90.58% | n.d. | n.d. | n.d. |
| 28 | KHFLALKPGQPMY | 100% | <90% | n.d. | n.d. | n.d. |
| 29 | KHDLRFKPGQPMY | 97.77% | <90% | n.d. | n.d. | n.d. |
| 30 | PHWLTIKPGQPMY | <90% | <90% | n.d. | n.d. | n.d. |
| 31 | KEWIQLKLGQPMY | <90% | <90% | n.d. | n.d. | n.d. |
| 32 | KSWLSLKPGQGMY | <90% | <90% | n.d. | n.d. | n.d. |
| 33 | WRWGQFKPGEPMY | <90% | <90% | n.d. | n.d. | n.d. |
| 34 | PHWLSFFPGQGMY | <90% | <90% | n.d. | n.d. | n.d. |
| 35 | KCWLELKTGMPMY | 97.61% | <90% | n.d. | n.d. | n.d. |
| 36 | WHMLEFKPGQPMY | <90% | <90% | n.d. | n.d. | n.d. |
| 37 | PHWIQLFNGQPMY | <90% | <90% | n.d. | n.d. | n.d. |
| 38 | KHVLQFKPGEPMY | <90% | <90% | n.d. | n.d. | n.d. |
| 39 | KSWGQFKPGQPMY | <90% | <90% | n.d. | n.d. | n.d. |
| 40 | PEWIQLKTGMPMY | <90% | <90% | n.d. | n.d. | n.d. |
| 41 | PHLLNFKPGQPMY | <90% | <90% | n.d. | n.d. | n.d. |
| 42 | PHSGQLWSGQPMY | <90% | <90% | n.d. | n.d. | n.d. |
| 43 | PHWGQFKLGQPMY | <90% | <90% | n.d. | n.d. | n.d. |
| 44 | PHWVNLKTGQGMY | <90% | <90% | n.d. | n.d. | n.d. |
| 45 | PTGLQFKPGQSMY | <90% | <90% | n.d. | n.d. | n.d. |
| 46 | WHWLSLNTGQPMY | 97.03% | <90% | n.d. | n.d. | n.d. |
| 47 | KHDLELWPGQPMY | <90% | <90% | n.d. | n.d. | n.d. |
| 48 | WGGLALKPGQPMY | <90% | <90% | n.d. | n.d. | n.d. |
| 49 | PEWLKLYPGQPMY | <90% | <90% | n.d. | n.d. | n.d. |
| 50 | WRVLQLKPGSSMY | <90% | <90% | n.d. | n.d. | n.d. |
| 51 | WHWVRIWPGQPMY | 100% | 100% | 97.13% | <90% | <90% |
| 52 | KHWLRIKWGEPMY | 100% | 100% | 100% | 97.66% | <90% |
| 53 | WKWVRLKTGQPMY | 100% | 100% | 100% | <90% | <90% |
| 54 | KHMIQLYWGQPMY | 100% | 100% | 93.21% | <90% | <90% |
| 55 | WRYLRLKPGSGMY | 100% | 100% | 99.43% | <90% | <90% |
| 56 | WHSLKLKRGQPMY | 100% | 100% | 99.84% | <90% | <90% |
| 57 | WHWLAIFRGQPMY | 100% | 100% | <90% | n.d. | 3.45% |
| 58 | WKWVQIKKGAPMY | 100% | 100% | <90% | n.d. | <90% |
| 59 | KHWITLKSGMPMY | 100% | 100% | 90.97% | <90% | <90% |
| 60 | WKTLQLKIGAPMY | 100% | 100% | <90% | n.d. | <90% |
| 61 | KHWLKLFPGQPMY | 100% | 100% | 98.64% | <90% | <90% |
| 62 | KHWLKIKIGEPMY | 100% | 100% | 100% | 98.89% | <90% |
| 63 | PRWLQIKKGQSMY | 100% | 100% | <90% | n.d. | <90% |
| 64 | KHWLQFKPGSGMY | 100% | 100% | 98.09% | <90% | <90% |
| 65 | WKWVNLRTGQPMY | 100% | 100% | <90% | n.d. | <90% |
| 66 | PHWISLKKGQPMY | 100% | 100% | 96.17% | <90% | <90% |
| 67 | KHMLRIKPGQPMY | 100% | 100% | 97.68% | <90% | <90% |
| 68 | KHWGRLRAGQPMY | 100% | 100% | 100% | 99.23% | 97.09% |
| 69 | KHWLRLNPGQPMY | 100% | 100% | 95.74% | <90% | <90% |
| 70 | WHYLRLKPGQSMY | 100% | 100% | <90% | n.d. | <90% |
| 71 | WHWIQLKKGEPMY | 100% | 100% | <90% | n.d. | <90% |
| 72 | KHYLKFKPGQPMY | 100% | 100% | 100% | 98.60% | <90% |
| 73 | WQWLRIKSGQGMY | 100% | 100% | <90% | n.d. | <90% |
| 74 | WHFVKLHPGQPMY | 100% | 100% | <90% | n.d. | <90% |
| 75 | KHWIRLWPGQPMY | 100% | 100% | 92.02% | <90% | <90% |
| 76 | WHWLRLKPGASMY | 100% | 100% | 97.59% | <90% | <90% |
| 77 | PHWIKLHTGQPMY | 99.74% | 100% | <90% | n.d. | <90% |
| 78 | KHFLNFKPGQPMY | 100% | 100% | <90% | n.d. | <90% |
| 79 | WRDIRLKKGQPMY | 100% | 100% | <90% | n.d. | <90% |
| 80 | KHTLRFKPGQPMY | 100% | 100% | 99.56% | <90% | <90% |
| 81 | KHWIKLKNGQPMY | 100% | 100% | 99.51% | <90% | <90% |
| 82 | WHWGSLKVGQPMY | 100% | 100% | <90% | n.d. | <90% |
| 83 | KHTLRLKPGQPMY | 100% | 100% | 99.92% | <90% | <90% |
| 84 | KHMLRFKPGQPMY | 100% | 100% | 97.08% | <90% | <90% |
| 85 | WHFLKFKPGEPMY | 100% | 100% | <90% | n.d. | <90% |
| 86 | KHFLKIKPGQPMY | 100% | 100% | 99.49% | <90% | <90% |
| 87 | KHVLKFKPGQPMY | 100% | 100% | 98.89% | <90% | <90% |
| 88 | KHLLRLKPGQPMY | 100% | 99.75% | <90% | n.d. | <90% |
| 89 | WHSLEFKIGQGMY | 100% | 99.55% | <90% | n.d. | <90% |
| 90 | WHFLQIKPGQGMY | 100% | 99.27% | <90% | n.d. | <90% |
| 91 | KQWLKLKNGQPMY | 100% | 98.66% | <90% | n.d. | <90% |
| 92 | WHTLRFKPGQPMY | 98.95% | 98.44% | <90% | n.d. | <90% |
| 93 | KHTLQLKRGQPMY | 100% | 97.77% | <90% | n.d. | <90% |
| 94 | KHYLSFKPGQPMY | 100% | 97.32% | <90% | n.d. | <90% |
| 95 | KHLLTFKPGQPMY | 99.99% | 97.12% | <90% | n.d. | <90% |
| 96 | KHFLTLKPGQPMY | 99.87% | 95.86% | <90% | n.d. | <90% |
| 97 | WHWVQFRPGAPMY | 100% | 95.75% | <90% | n.d. | <90% |
| 98 | WKWGSLKNGQPMY | 100% | 95.59% | <90% | n.d. | <90% |
| 99 | WHWLKIWPGQPMY | 100% | 94.66% | <90% | n.d. | <90% |
| 100 | WKYLNFKPGQPMY | 100% | 93.45% | <90% | n.d. | <90% |
| 101 | WHVLRFKPGQPMY | 100% | 90.50% | <90% | n.d. | <90% |
| 102 | KHYLTLKPGQPMY | 98.51% | <90% | <90% | n.d. | <90% |
| 103 | KHYLALKPGQPMY | 98.77% | <90% | <90% | n.d. | <90% |
| 104 | KRGINLKPGQPMY | 99.59% | <90% | <90% | n.d. | <90% |
| 105 | WHILRFKNGMPMY | 100% | <90% | <90% | n.d. | <90% |
| 106 | WHWVQLKKGEPMY | 100% | <90% | <90% | n.d. | <90% |
| 107 | KHFLSLKPGQPMY | 98.74% | <90% | <90% | n.d. | <90% |
| 108 | WHWLKLNYGQPMY | 100% | <90% | <90% | n.d. | <90% |
| 109 | KRWLQIRPGEPMY | 98.64% | <90% | <90% | n.d. | <90% |
| 110 | WHWLSIKPGQGMY | 97.94% | <90% | n.d. | n.d. | n.d. |
| 111 | WHWVQFGPGSPMY | 96.65% | <90% | <90% | n.d. | <90% |
| 112 | PHFLRLKPGQPMY | 99.57% | <90% | n.d. | n.d. | n.d. |
| 113 | KHLLNLKPGQPMY | 98.44% | <90% | n.d. | n.d. | n.d. |
| 114 | WHWINLWPGQPMY | 100% | <90% | n.d. | n.d. | n.d. |
| 115 | KKVLQLKLGQPMY | 99.72% | <90% | n.d. | n.d. | n.d. |
| 116 | KHDLKFKPGQPMY | 93.51% | <90% | n.d. | n.d. | n.d. |
| 117 | WQSGQLKPGQPMY | <90% | <90% | n.d. | n.d. | n.d. |
| 118 | WHMLNFKPGQPMY | <90% | <90% | n.d. | n.d. | n.d. |
| 119 | WHWLKLGTGEPMY | 98.65% | <90% | n.d. | n.d. | n.d. |
| 120 | WSMGQIKPGSPMY | <90% | <90% | n.d. | n.d. | n.d. |
| 121 | PSGLQIKVGQPMY | <90% | <90% | n.d. | n.d. | n.d. |
| 122 | PEWLSLKPGSSMY | <90% | <90% | n.d. | n.d. | n.d. |
| 123 | KQWVQLKYGQGMY | 100% | <90% | n.d. | n.d. | n.d. |
| 124 | WHGIQLFKGQPMY | 100% | <90% | n.d. | n.d. | n.d. |
| 125 | KEWVQLYPGQPMY | <90% | <90% | n.d. | n.d. | n.d. |
| 126 | PHDLEFKPGQGMY | <90% | <90% | n.d. | n.d. | n.d. |
| 127 | WKSINFKPGQPMY | <90% | <90% | n.d. | n.d. | n.d. |
| 128 | PHWVEFDPGQPMY | <90% | <90% | n.d. | n.d. | n.d. |
| 129 | WQGLELHPGQPMY | <90% | <90% | n.d. | n.d. | n.d. |
| 130 | WEVLTLKPGSPMY | <90% | <90% | n.d. | n.d. | n.d. |
| 131 | PHLIQLGLGQPMY | <90% | <90% | n.d. | n.d. | n.d. |
| 132 | PHMVRLKPGQPMY | 94.13% | <90% | n.d. | n.d. | n.d. |
| 133 | KGWGQLIPGEPMY | <90% | <90% | n.d. | n.d. | n.d. |
| 134 | WGDLKLKPGQPMY | <90% | <90% | n.d. | n.d. | n.d. |
| 135 | WHWGQLQRGEPMY | <90% | <90% | n.d. | n.d. | n.d. |
| 136 | PHWLKLKPGEPMY | <90% | <90% | n.d. | n.d. | n.d. |
| 137 | WHMGQLKPGEPMY | <90% | <90% | n.d. | n.d. | n.d. |
| 138 | WQMLKLKYGQPMY | <90% | <90% | n.d. | n.d. | n.d. |
| 139 | PHWGEIKPGQSMY | <90% | <90% | n.d. | n.d. | n.d. |
| 140 | KCFIQLKPGQPMY | 90.65% | <90% | n.d. | n.d. | n.d. |
| 141 | PRDLTLKRGQPMY | <90% | <90% | n.d. | n.d. | n.d. |
| 142 | PHGLSIYPGQPMY | <90% | <90% | n.d. | n.d. | n.d. |
| 143 | PHWGELKPGQPMY | <90% | <90% | n.d. | n.d. | n.d. |
| 144 | KHWVQLFPGQPMY | 99.99% | <90% | n.d. | n.d. | n.d. |
| 145 | KEWLNFKNGQPMY | <90% | <90% | n.d. | n.d. | n.d. |
| 146 | WRTGQFWPGQPMY | <90% | <90% | n.d. | n.d. | n.d. |
| 147 | PHDGQLKRGQPMY | <90% | <90% | n.d. | n.d. | n.d. |
| 148 | PKWLNLKPGEPMY | <90% | <90% | n.d. | n.d. | n.d. |
| 149 | WRVLKLMPGQPMY | <90% | <90% | n.d. | n.d. | n.d. |
| 150 | WHFLQIKPGEPMY | <90% | <90% | n.d. | n.d. | n.d. |
| 151 | KEWLNLKPGQPMY | <90% | <90% | n.d. | n.d. | n.d. |
| 152 | KSWLQIKNGQPMY | <90% | <90% | n.d. | n.d. | n.d. |
| 153 | KGILTLKPGQPMY | <90% | <90% | n.d. | n.d. | n.d. |
| 154 | KHVLALKPGSPMY | <90% | <90% | n.d. | n.d. | n.d. |
| 155 | WKILTIKPGQPMY | <90% | <90% | n.d. | n.d. | n.d. |
| 156 | KHDVQLKNGSPMY | <90% | <90% | n.d. | n.d. | n.d. |
| 157 | WHWGNFQTGQPMY | 97.14% | <90% | n.d. | n.d. | n.d. |
| 158 | PHWLNLKPGEPMY | <90% | <90% | n.d. | n.d. | n.d. |
| 159 | WKSLNFKPGQSMY | <90% | <90% | n.d. | n.d. | n.d. |
| 160 | PHWVSLKPGQPMY | <90% | <90% | n.d. | n.d. | n.d. |
| 161 | PHWVAFKPGSPMY | 90.52% | <90% | n.d. | n.d. | n.d. |
| 162 | KHYLELKPGQPMY | <90% | <90% | n.d. | n.d. | n.d. |
| 163 | WHMVQLKPGEPMY | <90% | <90% | n.d. | n.d. | n.d. |
| 164 | KGTLSLKPGQPMY | <90% | <90% | n.d. | n.d. | n.d. |
| 165 | KHSLEFKPGQPMY | <90% | <90% | n.d. | n.d. | n.d. |
| 166 | WEFVQFKRGQPMY | <90% | <90% | n.d. | n.d. | n.d. |
| 167 | WHWLEFQRGQPMY | <90% | <90% | n.d. | n.d. | n.d. |
| 168 | KHLVQLHPGQPMY | <90% | <90% | n.d. | n.d. | n.d. |
| 169 | PHTIQLWKGQPMY | <90% | <90% | n.d. | n.d. | n.d. |
| 170 | WHMLTFKPGQPMY | <90% | <90% | n.d. | n.d. | n.d. |
| 171 | WHLGQFKPGEPMY | <90% | <90% | n.d. | n.d. | n.d. |
| 172 | WSWGALKSGMPMY | <90% | <90% | n.d. | n.d. | n.d. |
| 173 | PHWVQLKNGQPMY | <90% | <90% | n.d. | n.d. | n.d. |
| 174 | WHSVQLHPGQPMY | <90% | <90% | n.d. | n.d. | n.d. |
| 175 | KKWIQLKPGEPMY | <90% | <90% | n.d. | n.d. | n.d. |
| 176 | WHDVNLKRGSPMY | <90% | <90% | n.d. | n.d. | n.d. |
| 177 | KHTLEFKPGQPMY | <90% | <90% | n.d. | n.d. | n.d. |
| 178 | WHSVQFWPGQPMY | 97.30% | <90% | n.d. | n.d. | n.d. |
| 179 | KHFLELKPGQPMY | <90% | <90% | n.d. | n.d. | n.d. |
| 180 | KHDLALKIGQPMY | <90% | <90% | n.d. | n.d. | n.d. |

¹n.d.; not determined

### Example 3: Peptide Antifungal Activity Towards Fusarium graminearum

The antifungal activity of alpha-factor peptide variants towards Fusarium graminearum will be tested.

### Experimental Procedure:

### a) Peptide Synthesis and Media

Peptides corresponding to SEQ ID NO: 1-303 and 326-374 and 401-420 are chemically synthesized and purified by HPLC, using a linear gradient of acetonitrile/water mixture, in a C-18 reverse phase HPLC (Agilent Technologies, USA). HPLC fractions are lyophilized and resuspended in nuclease-free water to 10 mM in Tris buffer, pH 7.6, and then diluted to 24 μM in the same buffer, from which a 1:1 dilution series is prepared.

### b) Preparation of CMC Broth

CMC broth, used for culturing Fusarium, is prepared as follows. An aliquot of 1 g yeast extract, 0.5 g MgSO4.7H2O, 1 g NH4NO3, and 1 g KH2PO4 are added to 700 ml of deionized water. Subsequently, an aliquot of 15 g of Carboxymethylcellulose (CMC) powder is added, while stirring continuously until the solution becomes clear and light-yellow in color. The volume is then adjusted to 1000 ml with additional deionized water, and autoclaved.

c) F. graminearum Cultivation and Spore Collection

An aliquot of frozen glycerol F. graminearum PH-1 spore suspension stock is thawed and 1020 μl is used to inoculate Potato dextrose agar (PDA) agar plates. Plates are incubated at 25° C. for a duration of 10 days. Conidia are produced by inoculating a 1 cm×1 cm mycelial plug from a 10-day old fungal culture in 25 ml of media in a 125 ml flat-bottom conical flask. The culture is incubated at 25° C. while shaking at 150 rpm for 3 days. Spore formation is checked under a microscope. The spore suspension is filtered through two layers of Miracloth, using Whatman Filter Holders or a funnel to remove mycelia, and the filtrate comprising the spores is collected in sterile 1.5 ml centrifuge tubes. The collected filtrate is centrifuged at 13,500 rpm for 1 minute, the pellet washed with sterile water, and finally the spores are resuspended in 2× Synthetic Fungal Medium (SFM). The suspension is adjusted to 1×10⁵ spores/ml using a hemocytometer.

### c) Antifungal Activity Towards Fusarium

In a microtiter plate, a 45 μl aliquot of the diluted peptides solutions is mixed with either 45 μl 2× SFM (media control) or with 45 μl 2× SFM comprising the 1×10⁵ Fusarium spores. The Fusarium growth control is spores mixed with buffer only (without peptide). A spectrophotometer reading is performed at 595 nm at 0 h. The plate is then sealed with parafilm to minimize evaporation and plates are incubated at 25° C. for 48 hours, after which absorbance is read again at 595 nm. The percent inhibition is calculated by reference to the non-treated Fusarium spores.

### d) Metabolic Activity of Treated Cells

At 48 hours, an aliquot of 20 μl of 0.05% resazurin solution is added to the microtiter plates containing the peptide-treated and untreated Fusarium suspension. The plates are sealed with parafilm and incubated at 25° C. for 18-22 hours, after which a color change from blue to pink/colorless, indicating metabolic activity, is assessed macroscopically and microscopically. The MIC value is determined from the well comprising the lowest peptide concentration at which there is no resazurin color change.

Antifungal Peptides corresponding to SEQ ID NO: 1-303 and 326-374 and 401-420 that show statistically significant growth inhibition against Fusarium are selected for formulation in antimicrobial composition or expression in a transgenic plant as described herein.

### Example 4: Peptide Antifungal Activity Towards Zymoseptoria tritici

This Example describes the antifungal activity of alpha-factor peptide variants towards Zymoseptoria tritici.

### Experimental Procedure:

### a) Peptide Synthesis and Media

Peptides corresponding to SEQ ID NO: 1-303 and 326-374 and 401-420 are chemically synthesized and purified by HPLC, using a linear gradient of acetonitrile/water mixture, in a C-18 reverse phase HPLC (Agilent Technologies, USA). HPLC fractions are lyophilized and resuspended in nuclease-free water to 10 mM in Tris buffer, pH 7.6, and then diluted to 24 μM in the same buffer, from which a 1:1 dilution series is prepared.

b) Zymoseptoria tritici Cultivation and Spore Collection

An aliquot of frozen glycerol Zymoseptoria tritici IPO323 spore suspension stock is thawed and 1020 μl is used to inoculate yeast peptone dextrose agar (YPD) agar plates. Plates are incubated at 18° C. for 3-4 days.

The plates are then gently flooded with 1-2 ml of sterile water. Then, using a sterile spreader or pipette tip, plates are scraped to scrape the surface to extract the fungal cells. The suspension is then filtered through two layers of Miracloth, using Whatman Filter Holders or a funnel to remove mycelia, and the filtrate, comprising the spores, is collected in sterile 1.5 ml centrifuge tubes. The collected filtrate is centrifuged at 13,500 rpm for 1 minute, the pellet washed with sterile water, and finally the spores are resuspended in 2× Synthetic Fungal Medium (SFM). The suspension is adjusted to 1×10⁵ spores/ml using a hemocytometer.

### c) Antifungal Activity Assessment

In a microtiter plate, a 45 μl aliquot of the diluted peptides solutions is mixed with either 45 μl 2× SFM (media control) or with 45 μl 2× SFM comprising the Zymoseptoria tritici spores. The Zymoseptoria tritici growth control is spores mixed with buffer only (without peptide). A spectrophotometer reading is performed at 595 nm at 0 h. The plate is then sealed with parafilm to minimize evaporation and incubated at 25° C. for 72 hours, after which absorbance is read again at 595 nm. The percent inhibition is calculated by reference to the non-treated Zymoseptoria tritici spores.

### d) Metabolic Activity Assessment

At 48 hours, an aliquot of 20 μl of 0.05% resazurin solution is added to the microtiter plates containing the peptide-treated and untreated Zymoseptoria tritici suspension. The plates are sealed with parafilm and incubated at 25° C. for 18-22 hours, after which a color change from blue to pink/colorless, indicating metabolic activity, is assessed macroscopically and microscopically. The MIC value is determined from the well comprising the lowest peptide concentration at which there is no resazurin color change.

Antifungal peptides corresponding to SEQ ID NO: 1-303 and 326-374 and 401-420 that show statistically significant growth inhibition against Zymoseptoria tritici are selected for formulation in antimicrobial composition or expression in a transgenic plant as described herein.

### Example 5. Peptide Antifungal Activity Towards Pseudoperonospora cubensis

This Example describes the antifungal activity of alpha-factor peptide variants towards Pseudoperonospora cubensis.

### Experimental Procedure:

### a) Peptide Synthesis and Media

Peptides corresponding to SEQ ID NO: 1-303 and 326-374 and 401-420 are chemically synthesized and purified by HPLC, using a linear gradient of acetonitrile/water mixture, in a C-18 reverse phase HPLC (Agilent Technologies, USA). HPLC fractions are lyophilized and resuspended in nuclease-free water to 10 mM in Tris buffer, pH 7.6, and then diluted to 24 μM in the same buffer, from which a 1:1 dilution series is prepared.

b) Antifungal Activity Towards Pseudoperonospora cubensis (Cucurbit Downy Mildew)—Leaf Disc Assay

Pseudoperonospora cubensis (cucurbit downy mildew) sporangia are harvested from leaves of infected cucurbit plants by gently running water over the leaf situated in a funnel within a conical tube so that the resulting suspension collected in the tube. This solution is passed through a 40-micrometer pluriStrainer filter, and sporangia in the filtrate are quantified using a hemocytometer.

A leaf disk assay is carried out in 12-well plates. Each well contained 4 milliliters water agar and a cucurbit leaf disk punched from healthy leaves using a hole punch of the same diameter as the well. Treatments are applied by spraying 1 milliliter of treatment solution using a hand-held spray brush. After plates are dried inside a fume hood, each leaf disc is inoculated with 1 milliliter of sporangia suspension, applied using the hand-held sprayer.

The readout of the cucurbit downy mildew leaf disk assay involved scoring each leaf disc according to a disease severity scale with 4 nominal ranges: 0%, 1-30%, 31-60%, and >60% disease. Discs are visually assessed, approximating sporangia/black structure coverage within the ranges. Treatment effectiveness is evaluated based on its ability to reduce disease severity compared to untreated controls.

Antifungal peptides corresponding to SEQ ID NO: 1-303 and 326-374 and 401-420 that show growth inhibition against Pseudoperonospora cubensis are selected for formulation in antimicrobial composition or expression in a transgenic plant as described herein.

### Example 6. Antifungal Activity of Peptides in Combination with FRAC Group 3 Fungicides Towards Fusarium graminearum

This Example describes the antifungal activity of alpha-factor peptide variants as described herein in combination with a FRAC group 3 fungicide, metoconazole, towards Fusarium graminearum.

### Experimental Procedure:

### a) Antimicrobial Peptides

Peptides corresponding to SEQ ID NO: 1-303 and 326-374 and 401-420 are synthesized by Genscript and stored at −20° C. Peptide stock solutions are prepared in MilliQ water at a final concentration of 1 mM. Stocks are aliquoted into cryostorage tubes and stored at −80° C. until use in the assay.

### b) Antimycotics

Metconazole is purchased from Sigma Aldrich, USA. Stocks are prepared in 100% DMSO (dimethyl sulfoxide) at a final concentration of 1 mg/mL. Stocks are stored at room temperature until used in the assay.

c) Isolation and Spore Collection of F. proliferatum

F. proliferatum isolated from corn seeds is used for all assays. To isolate F. proliferatum, corn seeds are soaked in water for at least an hour. The seeds are removed from the water and frozen at −20° C. overnight. They are then placed on plates of Fusarium-selective Nash and Snyder medium and incubated at room temperature. Once cultures have grown, a putative F. proliferatum isolate is chosen based on morphology and propagated on potato dextrose agar (PDA) (Difco, USA) at room temperature. DNA extraction and sequencing is then performed to confirm the isolate to be F. proliferatum.

Conidial stocks of F. proliferatum are prepared from 5 plates of 11-day-old cultures growing on PDA at room temperature. In a biosafety cabinet (BSC) about 3 mL of 0.01% Tween20 is added to each plate. A cell scraper is used to dislodge the conidia into suspension. The resulting liquid from each plate is passed through a 40-micron PluriSelect filter into a sterile 50 mL Falcon tube. Additionally, 0.01% Tween20 and 100% glycerol is added to the solution for a final volume of 20 mL at 20% glycerol. The concentration is quantified using a hemocytometer and is determined to be 1.39*10\hat{ }7 mg/mL. This solution is aliquoted into cryostorage tubes and stored at −80° C. until use in the assay.

d) Antifungal Activity Assessment Using oCelloscope Assay

Fungal inhibition assays are performed using the oCelloScope (Biosense Solutions, Denmark) to determine the concentration at which peptides and Metconazole alone demonstrated approximately 100%, 50%, and 0% inhibition of Fusarium proliferatum. The concentrations at which these phenotypes are observed are referred to as the treatments high (“H”, 100% inhibition), medium (“M”, 50% inhibition), and low concentrations (“L”, 0% inhibition). In these experiments, a treatment's relative minimum inhibitory concentration (MIC) is its high concentration. The same assay is then used to test peptides and Metconazole in various combination types to show a peptide's ability to reduce the MIC of Metconazole and other FRAC group 3 triazole fungicides of the same class.

Briefly, F. proliferatum conidia at a final concentration of 10,000 conidia/mL are co-incubated with peptide and Metconazole individually or in combination treatments in sterile 10% sucrose liquid media to a final volume of 150 microliters in 4 separate wells of a 96 well plate and incubated for 24 hours at 23° C. Images of each well are captured using the oCelloScope, and those images are visually reviewed for fungal growth and conidial germination to determine which conditions demonstrated inhibition of F. proliferatum.

To conduct the oCelloScope assay, all solutions including 10% sucrose media, F. proliferatum conidia stock, Metconazole stocks, and peptide stocks are brought to room temperature. Working stocks are prepared by performing dilutions of the original stocks in 10% sucrose. All conditions are prepared in individual microcentrifuge tubes by combining working stocks with media and conidia to achieve the final desired concentrations in a volume of 150 μl per replicate, with at least four replicates per condition. The solutions for each condition are then aliquoted, (150 μl in each well) into a sterile 96-well plate. Plates are incubated at 23° C. for 24 hours. An end point image is taken using the oCelloScope. These images are visually evaluated to determine conditions that demonstrated inhibition of F. proliferatum germination.

Alpha-factor variant peptides corresponding to SEQ ID NO: 1-303 and 326-374 and 401-420 that lower the Metaconazole dose required to achieve fungal growth inhibition are selected for formulation in antimicrobial composition or expression in a transgenic plant as described herein.

### Example 7. Antifungal Activity of Peptides in Combination with FRAC Group 48 Fungicides Towards Fusarium graminearum

This Example describes the antifungal activity of alpha-factor peptide variants as described herein in combination with a FRAC group 48 fungicide, Natamycin, towards Fusarium graminearum.

### Experimental Procedure:

### a) Antimicrobial Peptides

Peptides corresponding to SEQ ID NO: 1-303 and 326-374 and 401-420 are synthesized by Genscript and stored at −20° C. until stocks are prepared. These peptide stocks are prepared in MilliQ water at a final concentration of 1 mM. Stocks are aliquoted into cryostorage tubes and stored at −80° C. until use in the assay.

### b) Antimycotics

Natamycin is purchased from (Cayman Chemical Company, USA). Stocks are prepared in 100% DMSO (dimethyl sulfoxide) at a final concentration of 1 mg/mL. Stocks are stored at room temperature until used in the assay.

c) Isolation and Spore Collection of F. proliferatum

F. proliferatum isolated from corn seeds is used for all assays. To isolate F. proliferatum, corn seeds are soaked in water for at least an hour. The seeds are removed from the water and frozen at −20° C. overnight. They are then placed on plates of Fusarium-selective Nash and Snyder medium and incubated at room temperature. Once cultures have grown, a putative F. proliferatum isolate is chosen based on morphology and propagated on potato dextrose agar (PDA) (Difco, USA) at room temperature. DNA extraction and sequencing is then performed to confirm the isolate to be F. proliferatum.

Conidial stocks of F. proliferatum are prepared from 5 plates of 11-day-old cultures growing on PDA at room temperature. In a biosafety cabinet (BSC) about 3 mL of 0.01% Tween20 is added to each plate. A cell scraper is used to dislodge the conidia into suspension. The resulting liquid from each plate is passed through a 40-micron PluriSelect filter into a sterile 50 mL Falcon tube. Additionally, 0.01% Tween20 and 100% glycerol is added to the solution for a final volume of 20 mL at 20% glycerol. The concentration is quantified using a hemocytometer and is determined to be 1.39*10\hat{ }7 mg/mL. This solution is aliquoted into cryostorage tubes and stored at −80° C. until use in the assay.

d) Antifungal Activity Assessment Using oCelloscope Assay

Fungal inhibition assays are performed using the oCelloScope (Biosense Solutions, Denmark) to determine the concentration at which peptides and Natamycin alone demonstrated approximately 100%, 50%, and 0% inhibition of Fusarium proliferatum. The concentrations at which these phenotypes are observed are referred to as the treatments high (“H”, 100% inhibition), medium (“M”, 50% inhibition), and low concentrations (“L”, 0% inhibition). In these experiments, a treatment's relative minimum inhibitory concentration (MIC) is its high concentration. The same assay is then used to test peptides and Natamycin in various combination types to show a peptide's ability to reduce the MIC of Natamycin and other FRAC group 48 fungicides of the same class.

Briefly, F. proliferatum conidia at a final concentration of 10,000 conidia/mL are co-incubated with peptide and Natamycin individually or in combination treatments in sterile10% sucrose liquid media to a final volume of 150 microliters in 4 separate wells of a 96 well plate and incubated for 24 hours at 23° C. Images of each well are captured using the oCelloScope, and those images are visually reviewed for fungal growth and conidial germination to determine which conditions demonstrated inhibition of F. proliferatum.

To conduct the oCelloScope assay, all solutions including 10% sucrose media, F. proliferatum conidia stock, Natamycin stocks, and peptide stocks are brought to room temperature. Working stocks are prepared by performing dilutions of the original stocks in 10% sucrose. All conditions are prepared in individual microcentrifuge tubes by combining working stocks with media and conidia to achieve the final desired concentrations in a volume of 150 μl per replicate, with at least four replicates per condition. The solutions for each condition are then aliquoted, (150 μl in each well) into a sterile 96-well plate. Plates are incubated at 23° C. for 24 hours. An end point image is taken using the oCelloScope. These images are visually evaluated to determine conditions that demonstrated inhibition of F. proliferatum.

Antifungal peptides corresponding to SEQ ID NO: 1-303 and 326-374 and 401-420 that lower the Natamycin dose required to achieve fungal growth inhibition are selected for formulation in antimicrobial composition or expression in a transgenic plant as described herein.

### Example 8. Antifungal Activity of Peptides in Combination with Cell-Wall Interacting Fungicides Towards Fusarium graminearum

This Example describes the antifungal activity of alpha-factor peptide variants as described herein in combination with a cell-wall interacting fungicide, Drosomycin, towards Fusarium graminearum.

### Experimental Procedure:

### a) Antimicrobial Peptides

Peptides corresponding to SEQ ID NO: 1-303 and 326-374 and 401-420 are synthesized by Genscript and stored at −20° C. until stocks are prepared. The peptides are prepared in MilliQ water at a final concentration of 1 mM. Stocks are aliquoted into cryostorage tubes and stored at −80° C. until used in the assay.

### b) Antimycotics

Drosomycin is a 44-residue antifungal peptide with four intramolecular disulfide bridges, including a terminal cysteine, and is originally described from Drosophila flies; see Feldbaum et al. (1994) J. Biol Chem., 269:33159-33163. Drosomycin's sequence has homology with antifungal peptides identified from plants such as members of the Brassicaceae (Feldbaum et al. (1994)). Drosomycin-01 is heterologously expressed and detected as previously described, with modifications; see Yuan et al. (2006) Protein Expr. Purif., 52:457-462; doi: 10.1016/j.pep.2006.10.024. The coding sequence (Yuan et al. (2006)) is codon-optimized via De Novo DNA and cloned as an N-terminal translational fusion to glutathione S-transferase (GST), and the resulting plasmid transformed into E. coli cells, which are grown out into a 1-L culture. The culture is harvested and centrifuged. The cell pellet is frozen at −80° C., then thawed and lysed. The GST-Drosomycin-01 is purified from the cell lysate using a GST trapping column, on-column digestion is performed to remove the GST tag, and the eluted tagless drosomycin-01 is further purified by HPLC on a C18 reverse-phase column.

c) Isolation and Spore Collection of F. proliferatum

F. proliferatum isolated from corn seeds is used for all assays. To isolate F. proliferatum, corn seeds are soaked in water for at least an hour. The seeds are removed from the water and frozen at −20° C. overnight. They are then placed on plates of Fusarium-selective Nash and Snyder medium and incubated at room temperature. Once cultures have grown, a putative F. proliferatum isolate is chosen based on morphology and propagated on potato dextrose agar (PDA) (Difco, USA) at room temperature. DNA extraction and sequencing is then performed to confirm the isolate to be F. proliferatum.

Conidial stocks of F. proliferatum are prepared from 5 plates of 11-day-old cultures growing on PDA at room temperature. In a biosafety cabinet (BSC) about 3 mL of 0.01% Tween20 is added to each plate. A cell scraper is used to dislodge the conidia into suspension. The resulting liquid from each plate is passed through a 40-micron PluriSelect filter into a sterile 50 mL Falcon tube. Additionally, 0.01% Tween20 and 100% glycerol is added to the solution for a final volume of 20 mL at 20% glycerol. The concentration is quantified using a hemocytometer and is determined to be 1.39*10\hat{ }7 mg/mL. This solution is aliquoted into cryostorage tubes and stored at −80° C. until use in the assay.

d) Antifungal Activity Assessment Using oCelloscope Assay

Fungal inhibition assays are performed using the oCelloScope (Biosense Solutions, Denmark) to determine the concentration at which peptides and Drosomycin alone demonstrated approximately 100%, 50%, and 0% inhibition of Fusarium proliferatum. The concentrations at which these phenotypes are observed are referred to as the treatments high (“H”, 100% inhibition), medium (“M”, 50% inhibition), and low concentrations (“L”, 0% inhibition). In these experiments, a treatment's relative minimum inhibitory concentration (MIC) is its high concentration. The same assay is then used to test peptides and Drosomycin in various combination types to show a peptide's ability to reduce the MIC of Drosomycin and other fungicides of the same class.

Briefly, F. proliferatum conidia at a final concentration of 10,000 conidia/mL are co-incubated with peptide and Drosomycin individually or in combination treatments in sterile 10% sucrose liquid media to a final volume of 150 microliters in 4 separate wells of a 96 well plate and incubated for 24 hours at 23° C. Images of each well are captured using the oCelloScope, and those images are visually reviewed for fungal growth and conidial germination to determine which conditions demonstrated inhibition of F. proliferatum.

To conduct the oCelloScope assay, all solutions including 10% sucrose media, F. proliferatum conidia stock, Drosomycin stocks, and peptide stocks are brought to room temperature. Working stocks are prepared by performing dilutions of the original stocks in 10% sucrose. All conditions are prepared in individual microcentrifuge tubes by combining working stocks with media and conidia to achieve the final desired concentrations in a volume of 150 μl per replicate, with at least four replicates per condition. The solutions for each condition are then aliquoted, (150 μl in each well) into a sterile 96-well plate. Plates are incubated at 23° C. for 24 hours. An end point image is taken using the oCelloScope. These images are visually evaluated to determine conditions that demonstrated inhibition of F. proliferatum.

Alpha-factor peptide variants corresponding to SEQ ID NO: 1-303 and 326-374 and 401-420 that lower the Drosomycin dose required to achieve fungal growth inhibition are selected for formulation in antimicrobial composition or expression in a transgenic plant as described herein.

### Example 9. Antifungal Activity of Peptides in Combination with Fungicides that Inhibit Beta-Glucan Synthesis Towards Fusarium

This Example describes the antifungal activity of alpha-factor peptide variants as described herein in combination with a beta-glucan synthesis interacting fungicide, Caspofungin, towards Fusarium graminearum.

### Experimental Procedure:

### a) Antimicrobial Peptides

Peptides corresponding to SEQ ID NO: 1-303 and 326-374 and 401-420 are synthesized by Genscript and stored at −20° C. until stocks are prepared. The peptides are prepared in MilliQ water at a final concentration of 1 mM. Stocks are aliquoted into cryostorage tubes and stored at −80° C. until used in the assay.

### b) Antimycotics

The echninocandin fungicide caspofungin is tested. Stocks are stored at room temperature until used in the assay.

c) Isolation and Spore Collection of F. proliferatum

F. proliferatum isolated from corn seeds is used for all assays. To isolate F. proliferatum, corn seeds are soaked in water for at least an hour. The seeds are removed from the water and frozen at −20° C. overnight. They are then placed on plates of Fusarium-selective Nash and Snyder medium and incubated at room temperature. Once cultures had grown, a putative F. proliferatum isolate is chosen based on morphology and propagated on potato dextrose agar (PDA) (Difco, USA) at room temperature. DNA extraction and sequencing is then performed to confirm the isolate to be F. proliferatum.

Conidial stocks of F. proliferatum are prepared from 5 plates of 11-day-old cultures growing on PDA at room temperature. In a biosafety cabinet (BSC) about 3 mL of 0.01% Tween20 is added to each plate. A cell scraper is used to dislodge the conidia into suspension.

The resulting liquid from each plate is passed through a 40-micron PluriSelect filter into a sterile 50 mL Falcon tube. Additionally, 0.01% Tween20 and 100% glycerol is added to the solution for a final volume of 20 mL at 20% glycerol. The concentration is quantified using a hemocytometer and is determined to be 1.39*10\hat{ }7 mg/mL. This solution is aliquoted into cryostorage tubes and stored at −80° C. until use in the assay.

d) Antifungal Activity Assessment Using oCelloscope Assay

Fungal inhibition assays are performed using the oCelloScope (Biosense Solutions, Denmark) to determine the concentration at which peptides and caspofungin alone demonstrated approximately 100%, 50%, and 0% inhibition of Fusarium proliferatum. The concentrations at which these phenotypes are observed are referred to as the treatments high (“H”, 100% inhibition), medium (“M”, 50% inhibition), and low concentrations (“L”, 0% inhibition). In these experiments, a treatment's relative minimum inhibitory concentration (MIC) is its high concentration. The same assay is then used to test peptides and caspofungin in various combination types to show a peptide's ability to reduce the MIC of caspofungin and other fungicides of the same class.

Briefly, F. proliferatum conidia at a final concentration of 10,000 conidia/mL are co-incubated with peptide and caspofungin individually or in combination treatments in sterile10% sucrose liquid media to a final volume of 150 microliters in 4 separate wells of a 96 well plate and incubated for 24 hours at 23° C. Images of each well are captured using the oCelloScope, and those images are visually reviewed for fungal growth and conidial germination to determine which conditions demonstrated inhibition of F. proliferatum.

To conduct the oCelloScope assay, all solutions including 10% sucrose media, F. proliferatum conidia stock, caspofungin stocks, and peptide stocks are brought to room temperature. Working stocks are prepared by performing dilutions of the original stocks in 10% sucrose. All conditions are prepared in individual microcentrifuge tubes by combining working stocks with media and conidia to achieve the final desired concentrations in a volume of 150 μl per replicate, with at least four replicates per condition. The solutions for each condition are then aliquoted, (150 μl in each well) into a sterile 96-well plate. Plates are incubated at 23° C. for 24 hours. An end point image is taken using the oCelloScope. These images are visually evaluated to determine conditions that demonstrated inhibition of F. proliferatum.

Alpha-factor variant peptides corresponding to SEQ ID NO: 1-303 and 326-374 and 401-420 that lower the Caspofungin dose required to achieve fungal growth inhibition are selected for formulation in antimicrobial composition or expression in a transgenic plant as described herein.

### Example 10. Antifungal Activity of Peptides in Combination with Fungicides that Inhibit Signal Transduction Towards Fusarium graminearum

This Example describes the antifungal activity of alpha-factor peptide variants as described herein in combination with a fungicide that inhibits signal transduction, Fenpiclonil, towards Fusarium graminearum.

### Experimental Procedure:

### a) Antimicrobial Peptides

Peptides corresponding to SEQ ID NO: 1-303 and 326-374 and 401-420 are synthesized by Genscript and stored at −20° C. until stocks are prepared. The peptides are prepared in MilliQ water at a final concentration of 1 mM. Stocks are aliquoted into cryostorage tubes and stored at −80° C. until used in the assay.

### b) Antimycotics

Fenpiclonil and Fludioxonil are acquired from Sigma Aldrich. Fenpiclonil stocks are prepared in 100% acetone at a final concentration of 1 μM. Fludioxonil stocks are prepared in 100% dimethyl sulfoxide (DMSO) at a final concentration of 1 mg/mL. Stocks are stored at room temperature until used in the assay.

c) Isolation and Spore Collection of F. proliferatum

F. proliferatum isolated from corn seeds is used for all assays. To isolate F. proliferatum, corn seeds are soaked in water for at least an hour. The seeds are removed from the water and frozen at −20° C. overnight. They are then placed on plates of Fusarium-selective Nash and Snyder medium and incubated at room temperature. Once cultures had grown, a putative F. proliferatum isolate is chosen based on morphology and propagated on potato dextrose agar (PDA) (Difco, USA) at room temperature. DNA extraction and sequencing is then performed to confirm the isolate to be F. proliferatum.

Conidial stocks of F. proliferatum are prepared from 5 plates of 11-day-old cultures growing on PDA at room temperature. In a biosafety cabinet (BSC) about 3 mL of 0.01% Tween20 is added to each plate. A cell scraper is used to dislodge the conidia into suspension. The resulting liquid from each plate is passed through a 40-micron PluriSelect filter into a sterile 50 mL Falcon tube. Additionally, 0.01% Tween20 and 100% glycerol is added to the solution for a final volume of 20 mL at 20% glycerol. The concentration is quantified using a hemocytometer and is determined to be 1.39*10\hat{ }7 mg/mL. This solution is aliquoted into cryostorage tubes and stored at −80° C. until use in the assay.

d) Antifungal Activity Assessment Using oCelloscope Assay

Fungal inhibition assays are performed using the oCelloScope (Biosense Solutions, Denmark) to determine the concentration at which peptides and fenpiclonil or fludioxinil alone demonstrated approximately 100%, 50%, and 0% inhibition of Fusarium proliferatum. The concentrations at which these phenotypes are observed are referred to as the treatments high (“H”, 100% inhibition), medium (“M”, 50% inhibition), and low concentrations (“L”, 0% inhibition). In these experiments, a treatment's relative minimum inhibitory concentration (MIC) is its high concentration. The same assay is then used to test peptides and fenpiclonil or fludioxinil in various combination types to show a peptide's ability to reduce the MIC of fenpiclonil or fludioxinil and other FRAC group 3 triazole fungicides of the same class.

Briefly, F. proliferatum conidia at a final concentration of 10,000 conidia/mL are co-incubated with peptide and fenpiclonil or fludioxinil individually or in combination treatments in sterile10% sucrose liquid media to a final volume of 150 microliters in 4 separate wells of a 96 well plate and incubated for 24 hours at 23° C. Images of each well are captured using the oCelloScope, and those images are visually reviewed for fungal growth and conidial germination to determine which conditions demonstrated inhibition of F. proliferatum.

To conduct the oCelloScope assay, all solutions including 10% sucrose media, F. proliferatum conidia stock, fenpiclonil or fludioxinil stocks, and peptide stocks are brought to room temperature. Working stocks are prepared by performing dilutions of the original stocks in 10% sucrose. All conditions are prepared in individual microcentrifuge tubes by combining working stocks with media and conidia to achieve the final desired concentrations in a volume of 150 μl per replicate, with at least four replicates per condition. The solutions for each condition are then aliquoted, (150 μl in each well) into a sterile 96-well plate. Plates are incubated at 23° C. for 24 hours. An end point image is taken using the oCelloScope. These images are visually evaluated to determine conditions that demonstrated inhibition of F. proliferatum.

Alpha-factor variant peptides corresponding to SEQ ID NO: 1-303 and 326-374 and 401-420 that lower the Fenpiclonil dose required to achieve fungal growth inhibition for formulation in antimicrobial composition or expression in a transgenic plant as described herein.

### Example 11. Antifungal Activity of Peptides in Combination with Fungicides that Inhibit Fungal Growth or Reproduction Towards Fusarium graminearum

This Example describes the antifungal activity of alpha-factor peptide variants as described herein in combination with Boscalid or Trifloxystrobin, towards Fusarium graminearum.

### Experimental Procedure:

### a) Antimicrobial Peptides

Peptides corresponding to SEQ ID NO: 1-303 and 326-374 and 401-420 are synthesized by Genscript and stored at −20° C. until stocks are prepared. The peptides are prepared in MilliQ water at a final concentration of 1 mM. Stocks are aliquoted into cryostorage tubes and stored at −80° C. until used in the assay.

### b) Antimycotics

Boscalid and Trifloxystrobin are purchased Sigma, USA. Stocks are prepared in 100% DMSO (dimethyl sulfoxide) at a final concentration of 1 mg/mL. Stocks are stored at room temperature until used in the assay.

c) Isolation and Spore Collection of F. proliferatum

F. proliferatum isolated from corn seeds is used for all assays. To isolate F. proliferatum, corn seeds are soaked in water for at least an hour. The seeds are removed from the water and frozen at −20° C. overnight. They are then placed on plates of Fusarium-selective Nash and Snyder medium and incubated at room temperature. Once cultures had grown, a putative F. proliferatum isolate is chosen based on morphology and propagated on potato dextrose agar (PDA) (Difco, USA) at room temperature. DNA extraction and sequencing is then performed to confirm the isolate to be F. proliferatum.

Conidial stocks of F. proliferatum are prepared from 5 plates of 11-day-old cultures growing on PDA at room temperature. In a biosafety cabinet (BSC) about 3 mL of 0.01% Tween20 is added to each plate. A cell scraper is used to dislodge the conidia into suspension. The resulting liquid from each plate is passed through a 40-micron PluriSelect filter into a sterile 50 mL Falcon tube. Additionally, 0.01% Tween20 and 100% glycerol is added to the solution for a final volume of 20 mL at 20% glycerol. The concentration is quantified using a hemocytometer and is determined to be 1.39*10\hat{ }7 mg/mL. This solution is aliquoted into cryostorage tubes and stored at −80° C. until use in the assay.

d) Antifungal Activity Assessment Using oCelloscope Assay

Fungal inhibition assays are performed using the oCelloScope (Biosense Solutions, Denmark) to determine the concentration at which peptides, boscalid or trifloxystrobin alone demonstrated approximately 100%, 50%, and 0% inhibition of Fusarium proliferatum. The concentrations at which these phenotypes are observed are referred to as the treatments high (“H”, 100% inhibition), medium (“M”, 50% inhibition), and low concentrations (“L”, 0% inhibition). In these experiments, a treatment's relative minimum inhibitory concentration (MIC) is its high concentration. The same assay is then used to test peptides and boscalid or trifloxystrobin in various combination types to show a peptide's ability to reduce the MIC of boscalid or trifloxystrobin and other fungicides of the same class.

Briefly, F. proliferatum conidia at a final concentration of 10,000 conidia/mL are co-incubated with peptide and boscalid or trifloxystrobin individually or in combination treatments in sterile10% sucrose liquid media to a final volume of 150 microliters in 4 separate wells of a 96 well plate and incubated for 24 hours at 23° C. Images of each well are captured using the oCelloScope, and those images are visually reviewed for fungal growth and conidial germination to determine which conditions demonstrated inhibition of F. proliferatum.

To conduct the oCelloScope assay, all solutions including 10% sucrose media, F. proliferatum conidia stock, boscalid or trifloxystrobin stocks, and peptide stocks are brought to room temperature. Working stocks are prepared by performing dilutions of the original stocks in 10% sucrose. All conditions are prepared in individual microcentrifuge tubes by combining working stocks with media and conidia to achieve the final desired concentrations in a volume of 150 μl per replicate, with at least four replicates per condition. The solutions for each condition are then aliquoted, (150 μl in each well) into a sterile 96-well plate. Plates are incubated at 23° C. for 24 hours. An end point image is taken using the oCelloScope. These images are visually evaluated to determine conditions that demonstrated inhibition of F. proliferatum.

Alpha-factor variant peptides corresponding to SEQ ID NO: 1-303 and 326-374 and 401-420 that lower the Boscalid or Trifloxystrobin dose required to achieve fungal growth inhibition are selected for formulation in antimicrobial composition or expression in a transgenic plant as described herein.

### Example 12. Antifungal Activity of Peptides in Combination with Fungicides that Inhibit Amino Acid or Protein Synthesis Towards Fusarium

This Example describes the antifungal activity of alpha-factor peptide variants as described herein in combination with a fungicide that inhibits amino acid or protein synthesis, Cyprodinil, towards Fusarium graminearum.

### Experimental Procedure:

### a) Antimicrobial Peptides

Peptides corresponding to SEQ ID NO: 1-303 and 326-374 and 401-420 are synthesized by Genscript and stored at −20° C. until stocks are prepared. The peptides are prepared in MilliQ water at a final concentration of 1 mM. Stocks are aliquoted into cryostorage tubes and stored at −80° C. until used in the assay.

### b) Antimycotics

The anilinopyrimidine fungicide Cyprodinil is tested. Stocks are stored at room temperature until used in the assay.

c) Isolation and Spore Collection of F. proliferatum

F. proliferatum isolated from corn seeds is used for all assays. To isolate F. proliferatum, corn seeds are soaked in water for at least an hour. The seeds are removed from the water and frozen at −20° C. overnight. They are then placed on plates of Fusarium-selective Nash and Snyder medium and incubated at room temperature. Once cultures had grown, a putative F. proliferatum isolate is chosen based on morphology and propagated on potato dextrose agar (PDA) (Difco, USA) at room temperature. DNA extraction and sequencing is then performed to confirm the isolate to be F. proliferatum.

Conidial stocks of F. proliferatum are prepared from 5 plates of 11-day-old cultures growing on PDA at room temperature. In a biosafety cabinet (BSC) about 3 mL of 0.01% Tween20 is added to each plate. A cell scraper is used to dislodge the conidia into suspension.

The resulting liquid from each plate is passed through a 40-micron PluriSelect filter into a sterile 50 mL Falcon tube. Additionally, 0.01% Tween20 and 100% glycerol is added to the solution for a final volume of 20 mL at 20% glycerol. The concentration is quantified using a hemocytometer and is determined to be 1.39*10\hat{ }7 mg/mL. This solution is aliquoted into cryostorage tubes and stored at −80° C. until use in the assay.

d) Antifungal Activity Assessment Using oCelloscope Assay

Fungal inhibition assays are performed using the oCelloScope (Biosense Solutions, Denmark) to determine the concentration at which peptides and Cyprodinil alone demonstrated approximately 100%, 50%, and 0% inhibition of Fusarium proliferatum. The concentrations at which these phenotypes are observed are referred to as the treatments high (“H”, 100% inhibition), medium (“M”, 50% inhibition), and low concentrations (“L”, 0% inhibition). In these experiments, a treatment's relative minimum inhibitory concentration (MIC) is its high concentration. The same assay is then used to test peptides and cyprodinil in various combination types to show a peptide's ability to reduce the MIC of cyprodinil n and other fungicides of the same class.

Briefly, F. proliferatum conidia at a final concentration of 10,000 conidia/mL are co-incubated with peptide and cyprodinil individually or in combination treatments in sterile10% sucrose liquid media to a final volume of 150 microliters in 4 separate wells of a 96 well plate and incubated for 24 hours at 23° C. Images of each well are captured using the oCelloScope, and those images are visually reviewed for fungal growth and conidial germination to determine which conditions demonstrated inhibition of F. proliferatum.

To conduct the oCelloScope assay, all solutions including 10% sucrose media, F. proliferatum conidia stock, cyprodinil stocks, and peptide stocks are brought to room temperature. Working stocks are prepared by performing dilutions of the original stocks in 10% sucrose. All conditions are prepared in individual microcentrifuge tubes by combining working stocks with media and conidia to achieve the final desired concentrations in a volume of 150 μl per replicate, with at least four replicates per condition. The solutions for each condition are then aliquoted, (150 μl in each well) into a sterile 96-well plate. Plates are incubated at 23° C. for 24 hours. An end point image is taken using the oCelloScope. These images are visually evaluated to determine conditions that demonstrated inhibition of F. proliferatum.

Alpha-factor variant peptides corresponding to SEQ ID NO: 1-303 and 326-374 and 401-420 that lower the Cyprodinil dose required to achieve fungal growth inhibition are selected for formulation in antimicrobial composition or expression in a transgenic plant as described herein.

### Example 13. Antifungal Activity of Peptides in Combination with Macrolide Antifungals Towards Fusarium

The Example describes the effect of an alpha-factor variant peptide as described herein in combination with a macrolide antifungal agent, amphotericin B, against Fusarium oxysporum sp. heliotrophii.

### Experimental Procedure:

### a) Antimicrobial Peptides

Peptides corresponding to SEQ ID NO: 1-303 and 326-374 and 401-420 are synthesized by Genscript and stored at −20° C. until stocks are prepared. The peptides are prepared in MilliQ water at a final concentration of 1 mM. Stocks are aliquoted into cryostorage tubes and stored at −80° C. until used in the assay.

### b) Antimycotics

The fungicide Amphotericin B is tested. Stocks are stored at room temperature until used in the assay.

### c) Fusarium Cells

Fusarium conidia are prepared as described in previous examples. 1×10⁵ conidia/mL working solution from prepared from a −80° C. freezer stock in 1× potato dextrose broth (PDB).

### d) Experimental Procedure

Peptides corresponding to SEQ ID NO: 1-303 and 326-374 and 401-420 are chemically synthesized and tested in a 96 well microtiter plated in 1× PDB at a final concentration of 10 μM, alone or in combination with Amphotericin B (macrolide) at a final concentration of 2.5 μM. Metconazole (an azole, control) is tested at a final concentration of 375 μg/ml. Plates are incubated at 25° C. for 24 hrs after which 10 μL of Presto Blue reagent is added to each well on the plate. Plates are incubated for another 22 h at 25° C., incubator after which fluorescence is read at 570/620 nm with a read height of 7 mm.

Alpha-factor variant peptides corresponding to SEQ ID NO: 1-303 and 326-374 and 401-420 that lower the Amphotericin B dose required to achieve fungal growth inhibition are selected for formulation in antimicrobial composition or expression in a transgenic plant as described herein.

### Example 14. Antifungal Activity of Peptides Towards Botrytis on Pepper Plants

This example describes a lab-based tub assay for measuring the protective effect of alpha-factor variant peptides as described herein against gray mold disease development caused by Botrytis cinerea strain T-4 on California sweet pepper (Capsicum annuum cv. California)

### Experimental Procedure:

### a) Antimicrobial Peptides

Peptides corresponding to SEQ ID NO: 1-303 and 326-374 and 401-420 are synthesized by Genscript and stored at −20° C. until stocks are prepared. The peptides are prepared in MilliQ water at a final concentration of 1 mM. Stocks are aliquoted into cryostorage tubes and stored at −80° C. until used in the assay.

### b) Botrytis Spore Preparation

Botrytis cinerea strain T-4 conidia (spore) are used as the fungal inoculum. The fungus is grown in 20% V8 agar media for 2 weeks in dark at 25° C. The 2 weeks old media plates are flooded with 4-5 mL sterile distilled water to collect the fungal spores. The spore suspension is filtered through 2 layers of Mira cloth, centrifuged at 13,600 rpm for 1 min, washed 3 times with sterile distilled water, and re-suspended in ½ strength low-salt Synthetic Fungal Media (SFM). The spore suspension is adjusted to 10⁵ spores/mL using a hemocytometer.

### c) Experimental Treatments

The experimental preventative treatments are 0.5× SFM (buffer control),

0.02% Tween20 in 0.5× SFM (mock condition), Decree 0.5× (positive chemical control), Serenade 50 mL/L (positive biological control), and antimicrobial peptide at 20 μM (experimental).

### d) Preventative Peptide Treatments

Preventative treatment applications of the above solutions are performed 24 h prior to the fungal inoculation. Each treatment is applied at the rate of 2 mL per plant using a 10 ml capacity glass spray bottle. Once the treatments are applied, the plants are let on greenhouse bench for the treatments to dry out, which is approximately 1.5-2h. Meanwhile, 75-quart size Ziploc boxes, referred to as ‘tubs’, are lined with 2 layers of paper towel. In addition, twenty 5″ pots are set up to hold the plants from tripping. Once the treatments dried out, the plants are transferred to the tubs with 1 treatment per tub.

### e) Fungal Inoculation

On the day of inoculation, the plants are removed from the tubs and 500 mL of sterile distilled water is added to provide enough humidity required for disease development. Fungal spore suspension is prepared on the day of inoculation at 10⁵ spores/mL. An aliquot of 2 mL of the freshly prepared spore suspension is sprayed per plant inside the biosafety cabinet. The plants are then immediately transferred to the tubs and sealed immediately.

### f) Evaluation

The conditions are arranged in an RCBD design with 4 blocks per condition and 6 conditions are evaluated. The arrangement of conditions in an RCBD design is determined using ‘agricolae’ package in R version 4.3.2 using the following script.

The disease symptoms on 8 individual leaves, starting from the oldest leaf excluding cotyledon leaf, are visually assessed on a rating scale of 0-5 where each rank would represent the following proportion of diseased tissue per leaf:
Scale 0: No obvious signs/symptoms of disease
Scale 1:0>x≤20% diseased portion of leaf
Scale 2:20>x≤40% diseased portion of leaf
Scale 3:40>x≤60% diseased portion of leaf
Scale 4:60>x≤80% diseased portion of leaf
Scale 5:80>x≤100% diseased portion of leaf

In addition to the visual assessment, proportion of diseased tissue is assessed using auto imaging ML approach. High resolution images are captured at 72 h post spore inoculation. The images are then submitted to the bioinformatics team for ML based disease assessment.

Besides disease severity, inhibition of disease in each leaf for each of the treatments is calculated based on the baseline disease severity estimated for spore-only treatment across all the blocks. The following formula is used to assess the inhibition of disease severity by each treatment: Inhibition %=(Baseline DS-Treatment DS)/(Baseline DS)*100%, where Baseline DS is the average disease severity of spore-only treated leaves across all blocks, treatment DS is the disease severity for each leaf.

### g) Statistics

Descriptive statistics-count, minimum value, maximum value, average value, median value, standard deviation, and inter-quartile range (IQR)—are calculated for disease severity and inhibition percentage in R version 4.3.2. To assess the effect of blocking, the sources of variances are estimated by performing ANOVA in R. Once the block effects are estimated, an appropriate test is performed to compare treatment means against spore-only treatment. Plots are generated using ‘ggplot2’ package in R v 4.3.2

Alpha-factor variant peptides corresponding to SEQ ID NO: 1-303 and 326-374 and 401-420 that inhibit Botrytis on pepper plants are selected for formulation in antimicrobial composition or expression in a transgenic plant as described herein.

### Example 15. Antifungal Activity Testing Towards Botrytis cinerea

The antifungal activity of 23 alpha-factor peptide variants towards Botrytis was tested. Experimental design:

### a) Peptide Synthesis

Peptides corresponding to SEQ ID NO: 261-264 and 401-420 were chemically synthesized using the solid-phase synthesis method and resuspended in water to a stock solution of 1 mM. The stock solution was aliquoted in cryovials and stored at −80° C.

### b) Botrytis Culturing

The Botrytis cinerea culture (B05.10) isolate was acquired from the Centraalbureau voor Schimmelcultures, the Netherlands. The Botrytis fungus was propagated on 0.5% V8 agar plates (200 ml V8 Juice, 3 g CaCO3, 15 g agar, 800-ml millipore water) at 20° C., in a 12 h photoperiod for 21 days.

Fungal conidia were harvested by scraping a Botrytis plate flooded with 1 ml water with 0.1% Tween 80 solution. Conidia were then collected in a 50 ml falcon tube by passing the solution through a 40 micron filter. The solution was then centrifuged at 6000 rpm and the pellet was resuspended in 1 ml of 20% glycerol. The conidia concentration of the resulting solution was counted using a hemocytometer. The stock was diluted to 107 spores/ml using 20% glycerol and frozen in 100 μl aliquots in −80° C. until further use.

### c) Antifungal Assay

Peptides were tested for activity against Botrytis, using the resazurin assay. For this, fungal stock conidia was first diluted to 5000 conidia/well. 50 μl of the spore suspension was added to each well of the sterile 96-well clear bottom testing plate and mixed with 50 μl of peptide solutions to a final concentration of 100, 50, 20, 10, 5 and 2.5 μM. The plate was incubated for 24 h at 20° C. After 24 h, 10 μl of resazurin dye was added to the microtiter plate and incubated again for 20 h. Fluorescence was measured at 570/620 nm after 4 h of incubation with resazurin.

Growth inhibition was calculated by subtracting the fluorescence values of conidia only wells from sample wells and depicted as % inhibition of fungal growth. Four technical replicates were used for all treatments.

Results are shown in Table 6. Peptides that showed greater than 90% inhibition were considered active against Botrytis. 4 peptides, with SEQ ID NOs 261-264 were active at 5 μM. The peptides corresponding to SEQ ID NOs: 262 and 263 were active at 2.5 μM. In summary, a selection of the designed alpha-factor variants showed a remarkable antifungal activity at concentrations as low as 5 to 2.5 μM.

TABLE 6
Antifungal activity of 23 alpha factor peptides towards Botrytis

| SEQ ID | | Inhibition at1 | | | | | |
|---|---|---|---|---|---|---|---|
| NO: | Amino acid sequence | 100 μM | 50 μM | 20 μM | 10 μM | 5 μM | 2.5 μM |
| 261 | KHWGRLRAGQPMYGGGGKHW | n.d. | 100% | 100% | 100% | 99.3% | <90% |
| | GRLRAGQPMY | | | | | | |
| 262 | KHWGRLRAGQPMYGGGGRRR | n.d. | 100% | 100% | 100% | 100% | 99.8% |
| | RRRRR | | | | | | |
| 263 | KHWGRLRAGQPMYGGGGKKK | n.d. | 100% | 100% | 100% | 100% | 99.7% |
| | KKKKK | | | | | | |
| 264 | KHWGRLRAGQPMYGGGGYMP | n.d. | 100% | 100% | 100% | 96.2% | <90% |
| | QGARLRGWHK | | | | | | |
| 402 | KHILKFKPGQPMY | n.d. | 100% | 100% | 96.2% | <90% | <90% |
| 403 | KHYLRFKPGQPMY | n.d. | 100% | 100% | 96.0% | <90% | <90% |
| 404 | KHSLRLWPGQPMY | n.d. | 100% | <90% | <90% | n.d. | n.d. |
| 405 | KHWLTLFPGQPMY | n.d. | <90% | n.d. | n.d. | n.d. | n.d. |
| 406 | WQWLQLDRGQPMY | n.d. | <90% | n.d. | n.d. | n.d. | n.d. |
| 407 | WHSLQIKKGQPMY | n.d. | 100% | <90% | <90% | n.d. | n.d. |
| 408 | WHLLQFNWGQPMY | n.d. | 100% | <90% | <90% | n.d. | n.d. |
| 409 | WTVLTLKRGQSMY | n.d. | 100% | 100% | 97.7% | <90% | <90% |
| 410 | KHWLRFRPGQPMY | n.d. | 100% | 100% | <90% | n.d. | n.d. |
| 411 | KHWLRFWPGQPMY | n.d. | 100% | <90% | <90% | n.d. | n.d. |
| 412 | KHWLRIWPGQPMY | n.d. | 100% | <90% | <90% | n.d. | n.d. |
| 413 | KHWLRLWPGQPMY | n.d. | 100% | <90% | <90% | n.d. | n.d. |
| 414 | WHWLRLWPGQPMY | n.d. | <90% | n.d. | n.d. | n.d. | n.d. |
| 415 | WHWLQLKPGQPMYYMPQGPK | 100% | 100% | n.d. | n.d. | n.d. | n.d. |
| | LQLWHW | | | | | | |
| 416 | YMPQGPKLQLWHWWHWLQLK | 100% | 100% | n.d. | n.d. | n.d. | n.d. |
| | PGQPMY | | | | | | |
| 417 | WHWLQLKPGQPMYGGGGYMP | 100% | 100% | n.d. | n.d. | n.d. | n.d. |
| | QGPKLQLWHW | | | | | | |
| 418 | YMPQGPKLQLWHWGGGGWH | 100% | 100% | n.d. | n.d. | n.d. | n.d. |
| | WLQLKPGQPMY | | | | | | |
| 419 | RRRRRRRRWHWLQLKPGQPM | 100% | 100% | n.d. | n.d. | n.d. | n.d. |
| | Y | | | | | | |
| 420 | RRRRRRRRYMPQGPKLQLWH | 100% | 100% | n.d. | n.d. | n.d. | n.d. |
| | W | | | | | | |

¹n.d.; not determined

Although the foregoing disclosure describes aspects and embodiments of the invention in some detail by way of illustration and examples, the description and examples should not be construed as limiting the scope of the invention. Further embodiments are disclosed within the claims. The disclosures of all patent and scientific literature cited in this disclosure are expressly incorporated by reference in their entirety herein.

### OTHER EMBODIMENTS

1. A composition comprising:
   an effective amount of one or more antifungal peptides, or one or more polynucleotides encoding the one or more antifungal peptides, wherein the one or more antifungal peptides has an amino acid sequence with at least 80% sequence identity to a peptide selected from the group consisting of SEQ ID NOs 1-303 and 326-374 and 401-420.
2. The composition of embodiment 1, wherein the one or more antifungal peptides is selected from the group consisting of SEQ ID NO: 9, SEQ ID NO: 12, SEQ ID NO: 13, SEQ ID NO: 18, SEQ ID NO: 52, SEQ ID NO: 62, SEQ ID NO: 68, SEQ ID NO: 72, SEQ ID NO: 261, SEQ ID NO: 262, SEQ ID NO:263 and SEQ ID NO:264.
3. The composition of embodiment 1, wherein the two or more antifungal peptides are linked with a linker.
4. The composition of embodiment 1, wherein the linker is selected from the group consisting of GG and SEQ ID NO: 376-384, 399-400.
5. The composition of any of embodiments 1-4, wherein the composition further comprises at least one cell penetrating peptide (CPP), or a polynucleotide encoding for at least one CPP, wherein the CPP is selected from the group consisting of SEQ ID NO: 385-398.
6. The composition of embodiment 5, wherein the antimicrobial peptide and the CPP are fused.
7. The composition of embodiment 1, wherein the composition further comprises an antifungal agent.
8. The composition of embodiment 7, wherein the antifungal mechanism of the antimicrobial peptide and the antifungal mechanism of the antifungal agent differ from each other.
9. The composition of embodiment 7, wherein the antifungal agent is selected from the group consisting of a FRAC group 3, fungicide, a FRAC group 48 fungicide, a fungicide that interacts with the fungal cell wall, a fungicide that inhibits beta-glucan synthesis, a fungicide that inhibits signal transduction, a fungicide that inhibits fungal growth or reproduction, a fungicide that inhibits amino acid or protein synthesis, and a macrolide fungicide.
10. The composition of embodiment 9, wherein the macrolide fungicide is amphotericin B.
11. The composition of embodiment 1, wherein the one or more antifungal peptides is present at a concentration of between 1-200 μM.
12. The composition of embodiment 1, wherein the one or more antifungal peptides is present at a concentration of about 10 μM, about 15 μM, about 20 μM, about 50 μM or about 100 μM.
13. The composition of any of embodiments 1-12, wherein the composition further comprises a pharmaceutically acceptable carrier or an agriculturally acceptable carrier.
14. A method of decreasing growth or reproduction of a fungus, the method comprising providing a fungus with the antifungal composition comprising:
   an effective amount of one or more antifungal peptides, or one or more polynucleotides encoding for the one or more antifungal peptides operably linked to a heterologous promotor, wherein the one or more antifungal peptides has an amino acid sequence with at least 80% sequence identity to a peptide selected from the group consisting of SEQ ID NOs 1-303 and 326-374 and 401-420,
   thereby decreasing the growth or reproduction of the fungus.
15. The method of embodiment 14, wherein the one or more antifungal peptides is selected from the group consisting of SEQ ID NO: 9, SEQ ID NO: 12, SEQ ID NO: 13, SEQ ID NO: 18, SEQ ID NO: 52, SEQ ID NO: 62, SEQ ID NO: 68, SEQ ID NO: 72 SEQ ID NO: 261, SEQ ID NO:262, SEQ ID NO:263 and SEQ ID NO:264.
16. The method of embodiment 14, wherein the one or more antifungal peptides are linked with a linker.
17. The method of embodiment 14, wherein the linker is selected from the group consisting of GG and SEQ ID NO: 376-384, 399-400.
18. The method of any of embodiments 14-17, wherein the composition further comprises at least one cell penetrating peptide (CPP), or a polynucleotide encoding for at least one CPP, wherein the CPP is selected from the group consisting of SEQ ID NO: 385-398.
19. The method of embodiment 14, wherein the antimicrobial peptide and the CPP are fused.
20 The method of embodiment 14, wherein the composition further comprises an antifungal agent.
21. The method of embodiment 20, wherein the antifungal mechanism of the antimicrobial peptide and the antifungal mechanism of the antifungal agent differ from each other.
22. The method of embodiment 20, wherein the antifungal agent is selected from the group consisting of a FRAC group 3, fungicide, a FRAC group 48 fungicide, a fungicide that interacts with the fungal cell wall, a fungicide that inhibits beta-glucan synthesis, a fungicide that inhibits signal transduction, a fungicide that inhibits fungal growth or reproduction, a fungicide that inhibits amino acid or protein synthesis, and a macrolide fungicide.
23. The composition of embodiment 22, wherein the macrolide fungicide is amphotericin B.
24. The method of embodiment 14, wherein the one or more antifungal peptides is present at a concentration of between 1-200 μM.
25. The method of embodiment 14, wherein the one or more antifungal peptides is present at a concentration of about 10 μM, about 15 μM, about 20 M, about 50 μM or about 100 μM.
26. The method of any of embodiments 14-25, wherein the composition further comprises a pharmaceutically acceptable carrier or an agriculturally acceptable carrier.
27. The method of embodiment 14, wherein the composition is provided to the fungus by directly contacting the fungus with the composition, or by delivering the composition to the environment of the fungus.
28. The method of embodiment 14, wherein the polynucleotide encoding the one or more antifungal peptides is expressed in a fungus or in a plant.
29. The method of embodiment 14, wherein the fungus is a plant pathogen, a human pathogen, or an animal pathogen.
30. The method of embodiment 14, wherein the fungus is at least one selected from the group consisting of a Botrytis sp., a Fusarium sp., a Phytophthora sp., a Zymoseptoria sp., a Aspergillus sp., a Magnaporthe sp., a Puccinia sp., a Blumeria sp., a Mycosphaerella sp., a Colletotrichum sp., a Ustilago sp., a Melampsora sp., a Phakopsora sp., a Rhizoctonia sp., a Aspergillus sp., a Candida sp., a Coccidioides sp., a Histoplasma sp., a Cryptococcus sp., a Pneumocystis sp., and a Blastomyces sp.
31. The method of embodiment 30, wherein the fungus is Botrytis cinerea, Fusarium graminaerum, Fusarium oxysporum, Zymoseptoria tritici, Pseudoperonospora cubensis, Aspergillus fumigatus, or Candida albicans.
32. A method of reducing the dose of an antifungal agent used for treatment of an infection caused by a fungus in a subject, the method comprising administering to the subject a composition comprising an antifungal agent and one or more antifungal peptides selected from the group consisting of SEQ ID NO: 1-303 and 326-374 and 401-420.
33. The method of embodiment 32, wherein the one or more antifungal peptides is selected from the group consisting of SEQ ID NO: 9, SEQ ID NO: 12, SEQ ID NO: 13, SEQ ID NO: 18, SEQ ID NO: 52, SEQ ID NO: 62, SEQ ID NO: 68, SEQ ID NO: 72, SEQ ID NO: 261, SEQ ID NO: 262, SEQ ID NO:263 and SEQ ID NO:264.
34. The method of embodiment 32, wherein the antifungal agent is selected from the group consisting of a FRAC group 3, fungicide, a FRAC group 48 fungicide, a fungicide that interacts with the fungal cell wall, a fungicide that inhibits beta-glucan synthesis, a fungicide that inhibits signal transduction, a fungicide that inhibits fungal growth or reproduction, a fungicide that inhibits amino acid or protein synthesis, and a macrolide fungicide.
35. The composition of embodiment 34, wherein the macrolide fungicide is amphotericin B, and the fungus is C. albicans.
36 The method of embodiment 32, wherein the one or more antifungal peptides is present at a concentration of between 1-200 M.
37. The method of embodiment 32, wherein the one or more antifungal peptides is present at a concentration of about 10 μM, about 15 μM, about 20 μM, about 50 μM or about 100 μM.
38. The method of any of embodiments 32-37, wherein the composition further comprises a pharmaceutically acceptable carrier or an agriculturally acceptable carrier.
39 The method of embodiment 32, wherein the subject is a plant, a human or an animal.
40. The method of embodiment 32, wherein the subject is a plant, and wherein the composition is administered by foliar application.
41. The method of embodiment 32, wherein the subject is a human or an animal, and the composition is provided topically or orally.
42. A method of decreasing germ tube formation by a fungus, comprising providing a fungus with the antifungal composition comprising:
   an effective amount of one or more antifungal peptides, or one or more polynucleotides encoding for the one or more antifungal peptides operably linked to a heterologous promotor, wherein the one or more antifungal peptides has an amino acid sequence with at least 80% sequence identity to a peptide selected from the group consisting of SEQ ID NOs 1-303 and 326-374 and 401-420, whereby the germ tube formation by the fungus is decreased, relative to a control fungus not provided with the antifungal composition.
43. A method of preventing or reducing disease caused by a fungal pathogen of a plant, comprising providing to a plant the antifungal composition providing a fungus with the antifungal composition comprising:
   an effective amount of one or more antifungal peptides, or one or more polynucleotides encoding for the one or more antifungal peptides operably linked to a heterologous promotor, wherein the one or more antifungal peptides has an amino acid sequence with at least 80% sequence identity to a peptide selected from the group consisting of SEQ ID NOs 1-303 and 326-374 and 401-420,
whereby disease caused by the fungal pathogen is prevented or decreased in the plant, relative to a control plant not provided with the antifungal composition.
44. A method of treating a subject with or at risk of a disease caused by a fungus, comprising administering to the subject the antifungal composition providing a fungus with the antifungal composition comprising:
   an effective amount of one or more antifungal peptides, or one or more polynucleotides encoding for the one or more antifungal peptides operably linked to a heterologous promotor, wherein the one or more antifungal peptides has an amino acid sequence with at least 80% sequence identity to a peptide selected from the group consisting of SEQ ID NOs 1-303 and 326-374 and 401 and 420,
whereby the fungal disease is prevented or decreased in the subject, relative to a control subject not provided with the antifungal composition.
45 The method of embodiment 21, wherein the subject is:
   a) an animal selected from the group consisting of an invertebrate, an amphibian, a reptile, a bird, a cartilaginous or bony fish, and a non-human mammal; or
   b) a human.
46. A kit comprising:
   an effective amount of one or more antimicrobial peptides, or one or more polynucleotides encoding for the one or more antimicrobial peptides, wherein the one or more antimicrobial peptides has an amino acid sequence with at least 80% sequence identity to a peptide selected from the group consisting of SEQ ID NOs 1-303 and 326-374 and 401-420.
47. A composition comprising a Pichia pastoris cell comprising a polynucleotide encoding an alpha-factor variant peptide, wherein the polynucleotide is operably linked to a Pichia-expressible promoter and a secretion signal sequence, and wherein the alpha-factor variant antimicrobial peptide has at least 80% sequence identity to a peptide selected from the group consisting of SEQ ID NOs: 1-303 and 326-374 and 401-420.
48. A method of producing an alpha-factor variant peptide, the method comprising:
(a) transforming a Pichia pastoris cell with a polynucleotide encoding the peptide, wherein the peptide has at least 80% sequence identity to a peptide selected from the group consisting of SEQ ID NOs: 1-303 and 326-374 and 401-420;
(b) culturing the transformed Pichia pastoris cell under conditions suitable for expression; and
(c) recovering the expressed peptide from the culture medium.

1. A composition comprising:
an effective amount of one or more antifungal peptides, or one or more polynucleotides encoding the one or more antifungal peptides, wherein the one or more antifungal peptides has an amino acid sequence with at least 80% sequence identity to a peptide selected from the group consisting of SEQ ID NOs 1-303 and 326-374.

2. The composition of claim 1, wherein the one or more antifungal peptides is selected from the group consisting of SEQ ID NO: 9, SEQ ID NO: 12, SEQ ID NO: 13, SEQ ID NO: 18, SEQ ID NO: 52, SEQ ID NO: 62, SEQ ID NO: 68 and SEQ ID NO: 72.

3. The composition of claim 1, wherein the composition comprises an effective amount of two or more antifungal peptides, and wherein the two or more antifungal peptides are linked with a linker, and wherein the linker is selected from the group consisting of GG and SEQ ID NO: 376-384, 399-400.

4. (canceled)

5. The composition of claim 1, wherein the composition further comprises at least one cell penetrating peptide (CPP), or a polynucleotide encoding for at least one CPP, wherein the CPP is selected from the group consisting of SEQ ID NO: 385-398.

6. The composition of claim 5, wherein the antimicrobial peptide and the CPP are fused.

7. The composition of claim 1, wherein the composition further comprises an antifungal agent.

8. (canceled)

9. The composition of claim 7, wherein the antifungal agent is selected from the group consisting of a FRAC group 3 fungicide, a FRAC group 48 fungicide, a FRAC group 7 fungicide, a FRAC group 9 fungicide, a FRAC group 11 fungicide, a FRAC group 12 fungicide, a fungicide that interacts with the fungal cell wall, a fungicide that inhibits beta-glucan synthesis, a fungicide that inhibits signal transduction, a fungicide that inhibits amino acid or protein synthesis, and a macrolide fungicide.

10. The composition of claim 9, wherein the macrolide fungicide is amphotericin B.

11. The composition of claim 1, wherein each of the one or more antifungal peptides is present at a concentration of between 1-200 μM.

12. (canceled)

13. The composition of claim 1, wherein the composition further comprises a pharmaceutically acceptable carrier or an agriculturally acceptable carrier.

14. A method of decreasing growth or reproduction of a fungus, the method comprising providing a fungus with the antifungal composition comprising:
an effective amount of one or more antifungal peptides, or one or more polynucleotides encoding for the one or more antifungal peptides operably linked to a heterologous promotor, wherein the one or more antifungal peptides has an amino acid sequence with at least 80% sequence identity to a peptide selected from the group consisting of SEQ ID NOs 1-303 and 326-374,
thereby decreasing the growth or reproduction of the fungus.

15. The method of claim 14, wherein the one or more antifungal peptides is selected from the group consisting of SEQ ID NO: 9, SEQ ID NO: 12, SEQ ID NO: 13, SEQ ID NO: 18, SEQ ID NO: 52, SEQ ID NO: 62, SEQ ID NO: 68 and SEQ ID NO: 72.

16-19. (canceled)

20. The method of claim 14, wherein the composition further comprises an antifungal agent.

21. (canceled)

22. The method of claim 20, wherein the antifungal agent is selected from the group consisting of a FRAC group 3 fungicide, a FRAC group 48 fungicide, a FRAC group 7 fungicide, a FRAC group 9 fungicide, a FRAC group 11 fungicide, a FRAC group 12 fungicide, a fungicide that interacts with the fungal cell wall, a fungicide that inhibits beta-glucan synthesis, a fungicide that inhibits signal transduction, a fungicide that inhibits amino acid or protein synthesis, and a macrolide fungicide.

23. The method of claim 22, wherein the macrolide fungicide is amphotericin B.

24. The method of claim 14, wherein each of the one or more antifungal peptides is present at a concentration of between 1-200 μM.

25. (canceled)

26. The method of claim 14, wherein the composition further comprises a pharmaceutically acceptable carrier or an agriculturally acceptable carrier.

27. The method of claim 14, wherein the composition is provided to the fungus by directly contacting the fungus with the composition, or by delivering the composition to the environment of the fungus.

28. The method of claim 14, wherein the polynucleotide encoding the one or more antifungal peptides is expressed in a fungus or in a plant.

29. (canceled)

30. The method of claim 14, wherein the fungus is at least one selected from the group consisting of Botrytis sp., Fusarium sp., Phytophthora sp., Zymoseptoria sp., Aspergillus sp., Magnaporthe sp., Puccinia sp., Blumeria sp., Mycosphaerella sp., Colletotrichum sp., Ustilago sp., Melampsora sp., Phakopsora sp., Rhizoctonia sp., Aspergillus sp., Candida sp., Coccidioides sp., Histoplasma sp., Cryptococcus sp., Pneumocystis sp., and Blastomyces sp.

31-46. (canceled)
